(19) United States
(12) Patent Application Publication
(10) Pub. No.: US 2026/0193375 A1
(43) Pub. Date: Jul. 9, 2026

(54) ANTI-LY6K ANTIBODIES AND METHODS OF USE

(71) Applicant: Normunity, Inc., Boston, MA (US)

(72) Inventors: David ANDREW, Boston, MA (US); Jeff D. COLBERT, Boston, MA (US); William LAROCHELLE, Boston, MA (US); Rebecca J. NEWTH, Boston, MA (US); Richard SHIMKETS, Waltham, MA (US); Thomas VINCENT, Waltham, MA (US); Chew Shun CHANG, Waltham, MA (US)

(21) Appl. No.: 19/561,150

(22) Filed: Mar. 9, 2026

Related U.S. Application Data
(63) Continuation of application No. PCT/US2024/058421, filed on Dec. 4, 2024.
(60) Provisional application No. 63/605,977, filed on Dec. 4, 2023.
(60) Provisional application No. 63/662,231, filed on Jun. 20, 2024.
(60) Provisional application No. 63/686,563, filed on Aug. 23, 2024.

(51) Int. Cl. C07K 16/32; A61K 47/68; A61P 35/00; C07K 16/28

(52) U.S. Cl. CPC C07K 16/32; A61K 47/6851; A61P 35/00; C07K 16/2809; C07K 2317/31; C07K 2317/52; C07K 2317/565; C07K 2317/622; C07K 2317/77; C07K 2317/92

(57) ABSTRACT

The present disclosure provides antibodies and polypeptides that specifically bind to lymphocyte antigen 6 family member K (LY6K). Also provided are compositions comprising these antibodies, nucleic acids encoding these antibodies, expression vectors and host cells for making these antibodies, and methods of treating a subject using these antibodies.

### CROSS-REFERENCE TO RELATED APPLICATIONS

This application is a continuation of PCT Application No. PCT/US2024/058421, filed Dec. 4, 2024, which claims the benefit of and priority to U.S. Provisional Patent Application Ser. No. 63/605,977, filed Dec. 4, 2023; Ser. No. 63/662,231, filed Jun. 20, 2024; and Ser. No. 63/686,563, filed Aug. 23, 2024, the contents of which are hereby incorporated by reference in their entirety.

### REFERENCE TO SEQUENCE LISTING

This application contains a sequence listing which has been submitted electronically in ST.26 format and is hereby incorporated by reference in its entirety. Said ST.26 copy, created on Mar. 9, 2026, is named “360636-2016_seglist.xml” and is 223,434 bytes in size.

### FIELD

The present disclosure relates to antibodies that are specific for human lymphocyte antigen 6 family member K (LY6K), and methods of use thereof.

### BACKGROUND

The lymphocyte antigen 6 (LY6) family is a group of cysteine-rich proteins that share a common structure, are expressed in various tissues, and exhibit a wide variety of cellular functions. LY6 proteins are anchored to the cell membrane via glycosylphosphatidylinositol (GPI) anchor or secreted, and they are involved in cell proliferation, cell migration, cell-cell interactions, immune cell maturation, macrophage activation, and cytokine production. Dysregulated expression of LY6 family members is associated with tumorigenesis and autoimmune diseases, and many LY6 proteins are used as tumor markers and as potential therapeutic targets. Lymphocyte antigen 6 family member K (LY6K) is a cell surface protein that is upregulated in numerous types of cancer, including esophageal squamous cell carcinoma, bladder cancer, and breast cancer, and contributes to cell growth, migration, invasion, and immune escape.

Thus, there is a need for therapies targeting LY6K.

### SUMMARY

The present disclosure provides antibodies and polypeptides that specifically bind to LY6K (e.g., human LY6K). Also provided are pharmaceutical compositions comprising these antibodies, nucleic acids encoding these antibodies, expression vectors and host cells for making these antibodies, and methods of treating a subject using these antibodies. The antibodies provided herein are particularly advantageous because they specifically bind LY6K-expressing cancer cells, allowing targeted delivery of therapeutic and diagnostic agents, and therefore have utility in the treatment and diagnosis of cancer (e.g., LY6K-expressing cancer).

In one aspect, provided herein is an antibody that specifically binds human LY6K, the antibody comprising a VH comprising the CDRH1, CDRH2, and CDRH3 amino acid sequences of the VH amino acid sequence set forth in any one of SEQ ID NOs: 1-34 and a VL comprising the CDRL1, CDRL2, and CDRL3 amino acid sequences of the VL amino acid sequence set forth in any one of SEQ ID NOs: 35-68.

In certain embodiments, the VH amino acid sequence and VL amino acid sequence are as set forth in: SEQ ID NOs: 1 and 35, 2 and 36, 3 and 37, 4 and 38, 5 and 39, 6 and 40, 7 and 41, 8 and 42, 9 and 43, 10 and 44, 11 and 45, 12 and 46, 13 and 47, 14 and 48, 15 and 49, 16 and 50, 17 and 51, 18 and 52, 19 and 53, 20 and 54, 21 and 55, 22 and 56, 23 and 57, 24 and 58, 25 and 59, 26 and 60, 27 and 61, 28 and 62, 29 and 63, 30 and 64, 31 and 65, 32 and 66, 33 and 67, or 34 and 68, respectively.

In certain embodiments, the antibody comprises the CDRH1, CDRH2, and CDRH3 amino acid sequences set forth in: SEQ ID NOs: 69, 90, and 124; 70, 91, and 125; 71, 92, and 126; 72, 93, and 127; 69, 94, and 124; 73, 95, and 128; 74, 96, and 129; 75, 97, and 130; 74, 98, and 131; 76, 99, and 132; 77, 100, and 133; 78, 101, and 134; 79, 102, and 135; 74, 103, and 136; 80, 104, and 137; 74, 105, and 138; 74, 106, and 139; 81, 107, and 140; 81, 108, and 141; 82, 109, and 142; 83, 110, and 143; 84, 99, and 144; 85, 111, and 145; 81, 112, and 146; 86, 113, and 147; 87, 114, and 148; 74, 115, and 149; 74, 116, and 150; 74, 117, and 151; 88, 118, and 152; 89, 119, and 153; 81, 120, and 154; 74, 121, and 155; 85, 122, and 156; or 86, 123, and 157, respectively.

In certain embodiments, the antibody comprises the CDRL1, CDRL2, and CDRL3 amino acid sequences set forth in: SEQ ID NO: 158, KIS, and SEQ ID NO: 198; SEQ ID NO: 159, WAS, and SEQ ID NO: 199; SEQ ID NO: 160, STT, and SEQ ID NO: 200; SEQ ID NO: 161, WAS, and SEQ ID NO: 201; SEQ ID NO: 162, AAS, and SEQ ID NO: 202; SEQ ID NOs: 163, 185, and 203; SEQ ID NOs: 164, 185, and 204; SEQ ID NOs: 165, 185, and 205; SEQ ID NOs: 166, 186, and 206; SEQ ID NOs: 167, 187, and 207; SEQ ID NOs: 168, 188, and 208; SEQ ID NOs: 169, 186, and 209; SEQ ID NOs: 170, 185, and 198; SEQ ID NOs: 169, 189, and 210; SEQ ID NOs: 170, 185, and 211; SEQ ID NOs: 171, 187, and 212; SEQ ID NOs: 172, 187, and 213; SEQ ID NOs: 173, 190, and 214; SEQ ID NOs: 174, 191, and 215; SEQ ID NOs: 175, 186, and 216; SEQ ID NOs: 176, 186, and 217; SEQ ID NOs: 170, 192, and 198; SEQ ID NOs: 171, 193, and 218; SEQ ID NOs: 177, 187, and 219; SEQ ID NOs: 178, 189, and 220; SEQ ID NOs: 179, 187, and 221; SEQ ID NOs: 165, 185, and 222; SEQ ID NOs: 180, 187, and 223; SEQ ID NOs: 181, 194, and 224; SEQ ID NOs: 182, 195, and 199; SEQ ID NOs: 172, 187, and 225; SEQ ID NOs: 183, 196, and 226; or SEQ ID NOs: 184, 197, and 227, respectively.

In certain embodiments, the antibody comprises the CDRH1, CDRH2, CDRH3, CDRL1, CDRL2, and CDRL3 amino acid sequences set forth in: SEQ ID NOs: 69, 90, 124, 158, KIS, and SEQ ID NO: 198; SEQ ID NOs: 70, 91, 125, 159, WAS, and SEQ ID NO: 199; SEQ ID NOs: 71, 92, 126, 160, STT, and SEQ ID NO: 200; SEQ ID NOs: 72, 93, 127, 161, WAS, and SEQ ID NO: 201; SEQ ID NOs: 69, 94, 124, 158, KIS, and SEQ ID NO: 198; SEQ ID NOs: 73, 95, 128, 162, AAS, and SEQ ID NO: 202; SEQ ID NOs: 74, 96, 129, 163, 185, and 203; SEQ ID NOs: 75, 97, 130, 164, 185, and 204; SEQ ID NOs: 74, 98, 131, 165, 185, and 205; SEQ ID NOs: 76, 99, 132, 166, 186, and 206; SEQ ID NOs: 77, 100, 133, 167, 187, and 207; SEQ ID NOs: 78, 101, 134, 168, 188, and 208; SEQ ID NOs: 79, 102, 135, 169, 186, and 209; SEQ ID NOs: 74, 103, 136, 170, 185, and 198; SEQ ID NOs: 80, 104, 137, 169, 189, and 210; SEQ ID NOs: 74, 105, 138, 170, 185, and 211; SEQ ID NOs: 74, 106, 139, 171, 187, and 212; SEQ ID NOs: 81, 107, 140, 172, 187, and 213; SEQ ID NOs: 81, 108, 141, 173, 190, and 214; SEQ ID NOs: 82, 109, 142, 174, 191, and 215; SEQ ID NOs: 83, 110, 143, 175, 186, and 216; SEQ ID NOs: 84, 99, 144, 176, 186, and 217; SEQ ID NOs: 85, 111, 145, 170, 192, and 198; SEQ ID NOs: 81, 112, 146, 171, 193, and 218; SEQ ID NOs: 86, 113, 147, 177, 187, and 219; SEQ ID NOs: 87, 114, 148, 178, 189, and 220; SEQ ID NOs: 74, 115, 149, 179, 187, and 221; SEQ ID NOs: 74, 116, 150, 165, 185, and 222; SEQ ID NOs: 74, 117, 151, 180, 187, and 223; SEQ ID NOs: 88, 118, 152, 181, 194, and 224; SEQ ID NOs: 89, 119, 153, 182, 195, and 199; SEQ ID NOs: 81, 120, 154, 172, 187, and 225; SEQ ID NOs: 74, 121, 155, 165, 185, and 222; SEQ ID NOs: 85, 122, 156, 183, 196, and 226; or SEQ ID NOs: 86, 123, 157, 184, 197, and 227, respectively.

In certain embodiments, the antibody comprises the VH amino acid sequence set forth in any one of SEQ ID NOs: 1-34. In certain embodiments, the antibody comprises a heavy chain constant region, optionally selected from the group consisting of human IgG₁, IgG₂, IgG₃, IgG₄, IgA₁, and IgA₂. In certain embodiments, the antibody comprises a heavy chain constant region that is a variant of a wild-type heavy chain constant region, wherein the variant heavy chain constant region binds to an FcγR with lower affinity than the wild-type heavy chain constant region binds to the FcγR. In certain embodiments, the antibody comprises a heavy chain constant region comprising the amino acid sequence of SEQ ID NO: 228 or 229.

In certain embodiments, the antibody comprises the VL amino acid sequence set forth in any one of SEQ ID NOs: 35-68. In certain embodiments, the antibody comprises alight chain constant region comprising the amino acid sequence of SEQ ID NO: 230 or 231.

In certain embodiments, the VH and VL comprise the amino acid sequences set forth in: SEQ ID NOs: 1 and 35, 2 and 36, 3 and 37, 4 and 38, 5 and 39, 6 and 40, 7 and 41, 8 and 42, 9 and 43, 10 and 44, 11 and 45, 12 and 46, 13 and 47, 14 and 48, 15 and 49, 16 and 50, 17 and 51, 18 and 52, 19 and 53, 20 and 54, 21 and 55, 22 and 56, 23 and 57, 24 and 58, 25 and 59, 26 and 60, 27 and 61, 28 and 62, 29 and 63, 30 and 64, 31 and 65, 32 and 66, 33 and 67, or 34 and 68, respectively.

In another aspect, provided herein is an antibody that specifically binds to an amino acid sequence selected from the group consisting of SEQ ID NOs: 233-238. In another aspect, provided herein is an antibody that specifically binds to the amino acid sequence of SEQ ID NOs: 233 and 239. In another aspect, provided herein is an antibody that specifically binds to the amino acid sequence of SEQ ID NOs: 234 and 240. In another aspect, provided herein is an antibody that specifically binds to the amino acid sequence of SEQ ID NOs: 237 and 247.

In certain embodiments, an antibody provided herein is a multispecific antibody comprising a CD3-binding region, optionally wherein the CD3-binding region comprises a single-chain fragment variable (scFv) that specifically binds to CD3, optionally human CD3.

In another aspect, provided herein is a polypeptide comprising a VH comprising the CDRH1, CDRH2, and CDRH3 amino acid sequences of the VH amino acid sequence set forth in any one of SEQ ID NOs: 1-34. In certain embodiments, the VH comprises the CDRH1, CDRH2, and CDRH3 amino acid sequences set forth in SEQ ID NOs: 69, 90, and 124; 70, 91, and 125; 71, 92, and 126; 72, 93, and 127; 69, 94, and 124; 73, 95, and 128; 74, 96, and 129; 75, 97, and 130; 74, 98, and 131; 76, 99, and 132; 77, 100, and 133; 78, 101, and 134; 79, 102, and 135; 74, 103, and 136; 80, 104, and 137; 74, 105, and 138; 74, 106, and 139; 81, 107, and 140; 81, 108, and 141; 82, 109, and 142; 83, 110, and 143; 84, 99, and 144; 85, 111, and 145; 81, 112, and 146; 86, 113, and 147; 87, 114, and 148; 74, 115, and 149; 74, 116, and 150; 74, 117, and 151; 88, 118, and 152; 89, 119, and 153; 81, 120, and 154; 74, 121, and 155; 85, 122, and 156; or 86, 123, and 157, respectively.

Also provided herein is a polypeptide comprising a VL comprising the CDRL1, CDRL2, and CDRL3 amino acid sequences of the VL amino acid sequence set forth in any one of SEQ ID NOs: 35-68. In certain embodiments, the VL comprises the CDRL1, CDRL2, and CDRL3 amino acid sequences set forth in SEQ ID NO: 158, KIS, and SEQ ID NO: 198; SEQ ID NO: 159, WAS, and SEQ ID NO: 199; SEQ ID NO: 160, STT, and SEQ ID NO: 200; SEQ ID NO: 161, WAS, and SEQ ID NO: 201; SEQ ID NO: 162, AAS, and SEQ ID NO: 202; SEQ ID NOs: 163, 185, and 203; SEQ ID NOs: 164, 185, and 204; SEQ ID NOs: 165, 185, and 205; SEQ ID NOs: 166, 186, and 206; SEQ ID NOs: 167, 187, and 207; SEQ ID NOs: 168, 188, and 208; SEQ ID NOs: 169, 186, and 209; SEQ ID NOs: 170, 185, and 198; SEQ ID NOs: 169, 189, and 210; SEQ ID NOs: 170, 185, and 211; SEQ ID NOs: 171, 187, and 212; SEQ ID NOs: 172, 187, and 213; SEQ ID NOs: 173, 190, and 214; SEQ ID NOs: 174, 191, and 215; SEQ ID NOs: 175, 186, and 216; SEQ ID NOs: 176, 186, and 217; SEQ ID NOs: 170, 192, and 198; SEQ ID NOs: 171, 193, and 218; SEQ ID NOs: 177, 187, and 219; SEQ ID NOs: 178, 189, and 220; SEQ ID NOs: 179, 187, and 221; SEQ ID NOs: 165, 185, and 222; SEQ ID NOs: 180, 187, and 223; SEQ ID NOs: 181, 194, and 224; SEQ ID NOs: 182, 195, and 199; SEQ ID NOs: 172, 187, and 225; SEQ ID NOs: 183, 196, and 226; or SEQ ID NOs: 184, 197, and 227, respectively.

Also provided herein is a polypeptide comprising the amino acid sequence set forth in any one of SEQ ID NOs: 1-68.

In certain embodiments, an antibody or polypeptide provided herein is conjugated to a cytotoxic agent, cytostatic agent, toxin, radionuclide, or detectable label.

In another aspect, provided herein is a polynucleotide encoding: a VH, a VL, a heavy chain, and/or a light chain of an antibody or a polypeptide described herein. Also provided is a vector comprising a polynucleotide described herein. Also provided is a recombinant host cell comprising a polynucleotide described herein; a vector described herein; a polynucleotide encoding a heavy chain variable region or a heavy chain of an antibody described herein and a second polynucleotide encoding a light chain variable region or a light chain of an antibody described herein; and/or a first vector comprising a first polynucleotide encoding a heavy chain variable region or a heavy chain of an antibody described herein and a second vector comprising a second polynucleotide encoding a light chain variable region or a light chain of an antibody described herein.

In another aspect provided herein is a composition comprising an antibody, a polypeptide, a polynucleotide, a vector, and/or a host cell described herein and a pharmaceutically acceptable carrier or excipient.

In another aspect, provided herein is a method of producing an antibody, the method comprising culturing a host cell described herein under suitable conditions such that a polynucleotide is expressed, and the antibody is produced.

Also provided herein is a method of treating cancer in a subject, the method comprising administering to the subject an effective amount of: an antibody described herein; a polypeptide described herein; a polynucleotide described herein; a vector described herein; a host cell described herein; and/or a composition described herein.

### BRIEF DESCRIPTION OF THE DRAWINGS

FIG. 1 shows that exemplary anti-LY6K antibodies provided herein selectively bind LY6K, according to aspects of the present disclosure. Depicted are the plate reading results of ELISA assays measuring the binding of the indicated anti-LY6K antibodies to LY6K, LY6E, LY6D, LY6H, and BSA (negative control).

FIG. 2 shows that exemplary anti-LY6K antibodies provided herein detect LY6K, according to aspects of the present disclosure. Depicted in the left panel are the results of a capture ELISA measuring the sensitivity of the indicated anti-LY6K antibodies, as well as positive control commercially available anti-LY6K antibodies from R&D Systems and Abcam, for detecting LY6K. The table in the right panel indicates the sensitivity and half maximal effective concentration (EC50) for each of the indicated antibodies.

FIG. 3 shows that an anti-LY6K antibody provided herein is internalized into cells, according to aspects of the present disclosure. Depicted is a representative fluorescence plot for anti-LY6K antibody 3082 labeled with pH-sensitive dye. Upon internalization into OVCAR-8 cells, the antibody is endocytosed and releases fluorescent signal. Also depicted are results for negative control huIgG1 and positive control trastuzumab, a known commercially available internalizer.

FIG. 4 shows that an anti-LY6K antibody provided herein detects LY6K, according to aspects of the present disclosure. Depicted are representative IHC images showing detection of LY6K in OVCAR-8 cells, 293 LY6K cells, and 293 cells (negative control) by anti-LY6K antibody 2660, a commercially available positive control anti-LY6K antibody (Abcam), and IgG negative control.

FIG. 5 shows that anti-LY6K bispecific T cell engagers provided herein kill tumor cells, according to aspects of the present disclosure. Depicted is a plot showing percent killing of OVCAR-8 tumor cells by addition of increasing concentrations of L2K formatted anti-LY6K bispecific T cell engagers based on the indicated anti-LY6K antibodies (3785, 3520, 2660, 3741) and of a 4D5-based 1+1 T cell engager against Her2 as a comparison.

### DETAILED DESCRIPTION

The instant disclosure provides anti-LY6K antibodies and polypeptides. Also provided are pharmaceutical compositions comprising these antibodies, nucleic acids encoding these antibodies, expression vectors and host cells for making these antibodies, and methods of treating a subject using these antibodies. The antibodies disclosed herein are particularly useful for diagnosing and/or treating cancer in a subject.

### Definitions

As used herein, the term “LY6K” refers to lymphocyte antigen 6 family member K, a membrane-anchored protein that is highly expressed in cancer cells and tissues. As used herein, the term “human LY6K” refers to a protein encoded by a wild-type human LY6K gene (e.g., the gene set forth in NM_017527.4). The amino acid sequence of an exemplary human LY6K protein (as set forth in RefSeq NP_059997.3) is

(SEQ ID NO: 232)
MALLALLLVVALPRVWTDANLTARQRDPEDSQRTDEGDNRVWCHVC
ERENTFECQNPRRCKWTEPYCVIAAVKIFPRFFMVAKQCSAGCAAM
ERPKPEEKRFLLEEPMPFFYLKCCKIRYCNLEGPPINSSVFKEYAG
SMGESCGGLWLAILLLLASIAAGLSLS.

As used herein, the terms “antibody” and “antibodies” include full-length antibodies, antigen-binding fragments of full-length antibodies, and molecules comprising antibody CDRs, VH regions, and/or VL regions. Examples of antibodies include, without limitation, monoclonal antibodies, recombinantly produced antibodies, monospecific antibodies, multispecific antibodies (including bispecific antibodies), human antibodies, humanized antibodies, chimeric antibodies, immunoglobulins, synthetic antibodies, tetrameric antibodies comprising two heavy chain and two light chain molecules, an antibody light chain monomer, an antibody heavy chain monomer, an antibody light chain dimer, an antibody heavy chain dimer, an antibody light chain-antibody heavy chain pair, intrabodies, heteroconjugate antibodies, antibody-drug conjugates, bispecific T cell engagers (BiTEs), chimeric antigen receptors, single domain antibodies, monovalent antibodies, single-chain antibodies or single-chain Fvs (scFv), camelized antibodies, affibodies, Fab fragments, F(ab′)₂ fragments, disulfide-linked Fvs (sdFv), anti-idiotypic (anti-Id) antibodies (including, e.g., anti-anti-Id antibodies), and antigen-binding fragments of any of the above. In certain embodiments, antibodies described herein refer to polyclonal antibody populations. Antibodies can be of any type (e.g., IgG, IgE, IgM, IgD, IgA, or IgY), any class (e.g., IgG₁, IgG₂, IgG₃, IgG₄, IgA₁, or IgA₂), or any subclass (e.g., IgG₂a or IgG₂b) of immunoglobulin molecule. In certain embodiments, antibodies described herein are IgG antibodies, or a class (e.g., human IgG₁ or IgG₄) or subclass thereof. In a specific embodiment, the antibody is a humanized monoclonal antibody. In another specific embodiment, the antibody is a human monoclonal antibody.

“Multispecific antibodies” are antibodies (e.g., bispecific antibodies) that specifically bind to two or more different antigens or two or more different regions of the same antigen. Multispecific antibodies include bispecific antibodies that contain two different antigen-binding sites (exclusive of the Fc region). Multispecific antibodies can include, for example, recombinantly produced antibodies, human antibodies, humanized antibodies, resurfaced antibodies, chimeric antibodies, immunoglobulins, synthetic antibodies, tetrameric antibodies comprising two heavy chain and two light chain molecules, an antibody light chain monomer, heteroconjugate antibodies, linked single-chain antibodies or linked-single-chain Fvs (scFv), camelized antibodies, affybodies, linked Fab fragments, F(ab′)₂ fragments, chemically-linked Fvs, and disulfide-linked Fvs (sdFv). Multispecific antibodies can be of any type (e.g., IgG, IgE, IgM, IgD, IgA, or IgY), any class (e.g., IgG₁, IgG₂, IgG₃, IgG₄, IgA₁, or IgA₂), or any subclass (e.g., IgG₂a or IgG₂b) of immunoglobulin molecule. In certain embodiments, multispecific antibodies described herein are IgG antibodies, or a class (e.g., human IgG₁, IgG₂, or IgG₄) or subclass thereof.

As used herein, the term “CDR” or “complementarity determining region” means the noncontiguous antigen combining sites found within the variable regions of heavy and light chain polypeptides. These particular regions have been described by, for example, Kabat et al., J. Biol. Chem. 252, 6609-6616 (1977) and Kabat et al., Sequences of protein of immunological interest. (1991), by Chothia et al., J. Mol. Biol. 196: 901-917 (1987), and by MacCallum et al., J. Mol. Biol. 262: 732-745 (1996), all of which are herein incorporated by reference in their entireties, where the definitions include overlapping or subsets of amino acid residues when compared against each other. In certain embodiments, the term “CDR” is a CDR as defined by MacCallum et al., J. Mol. Biol. 262:732-745 (1996) and Martin A. “Protein Sequence and Structure Analysis of Antibody Variable Domains,” in Antibody Engineering, Kontermann and Dubel, eds., Chapter 31, pp. 422-439, Springer-Verlag, Berlin (2001). In certain embodiments, the term “CDR” is a CDR as defined by Kabat et al., J. Biol. Chem. 252, 6609-6616 (1977) and Kabat et al., Sequences of protein of immunological interest. (1991). In certain embodiments, heavy chain CDRs and light chain CDRs of an antibody are defined using different conventions. In certain embodiments, heavy chain CDRs and/or light chain CDRs are defined by performing structural analysis of an antibody and identifying residues in the variable region(s) predicted to make contact with an epitope region of a target molecule (e.g., human LY6K). CDRH1, CDRH2, and CDRH3 denote the heavy chain CDRs, and CDRL1, CDRL2, and CDRL3 denote the light chain CDRs.

As used herein, the terms “variable region” and “variable domain” are used interchangeably and are common in the art. The variable region typically refers to a portion of an antibody, generally, a portion of a light or heavy chain, typically about the amino-terminal 110 to 120 amino acids or 110 to 125 amino acids in the mature heavy chain and about 90 to 115 amino acids in the mature light chain, which differ extensively in sequence among antibodies and are used in the binding and specificity of a particular antibody for its particular antigen. The variability in sequence is concentrated in those regions called complementarity determining regions (CDRs) while the more highly conserved regions in the variable region are called framework regions (FRs). Without wishing to be bound by any particular mechanism or theory, it is believed that the CDRs of the light and heavy chains are primarily responsible for the interaction and specificity of the antibody with antigen. In certain embodiments, the variable region is a human variable region. In certain embodiments, the variable region comprises rodent or murine CDRs and human framework regions (FRs). In certain embodiments, the variable region is a primate (e.g., non-human primate) variable region. In certain embodiments, the variable region comprises rodent or murine CDRs and primate (e.g., non-human primate) framework regions (FRs).

As used herein, the terms “VH” and “VL” refer to antibody heavy and light chain variable regions, respectively, as described in Kabat et al., (1991) Sequences of Proteins of Immunological Interest (NIH Publication No. 91-3242, Bethesda), which is herein incorporated by reference in its entirety.

As used herein, the term “constant region” is common in the art. The constant region is an antibody portion, e.g., a carboxyl terminal portion of a light and/or heavy chain, which is not directly involved in binding of an antibody to antigen but which can exhibit various effector functions, such as interaction with an Fc receptor (e.g., Fc gamma receptor).

As used herein, the term “heavy chain” when used in reference to an antibody can refer to any distinct type, e.g., alpha (α), delta (δ), epsilon (ε), gamma (γ), and mu (μ), based on the amino acid sequence of the constant region, which give rise to IgA, IgD, IgE, IgG, and IgM classes of antibodies, respectively, including subclasses of IgG, e.g., IgG₁, IgG₂, IgG₃, and IgG₄.

As used herein, the term “light chain” when used in reference to an antibody can refer to any distinct type, e.g., kappa (κ) or lambda (λ), based on the amino acid sequence of the constant region. Light chain amino acid sequences are well known in the art. In specific embodiments, the light chain is a human light chain.

As used herein, the term “specifically binds” refers to the specificity of a binding molecule (e.g., an antibody) for an antigen, as is understood by one skilled in the art. Binding molecules that specifically bind to an antigen typically bind to the antigen with an equilibrium dissociation constant (KD) of less than 1×10⁻⁶ M, as measured by, e.g., ELISA assay, surface plasmon resonance, or other suitable assays known in the art. The skilled worker will appreciate that, in certain embodiments, a binding molecule can specifically bind to different antigens, e.g., different antigens that share a common epitope that is recognized by the binding molecule.

As used herein, the term “affinity” refers to the strength of the sum total of noncovalent interactions between a single binding site of a molecule (e.g., an antibody) and its binding partner (e.g., an antigen). Unless indicated otherwise, as used herein, “binding affinity” refers to intrinsic binding affinity which reflects a 1:1 interaction between members of a binding pair (e.g., antibody and antigen). The affinity of a molecule X for its partner Y can generally be represented by the dissociation constant (KD). Affinity can be measured by common methods known in the art, including those described herein.

As used herein, the term “EU numbering system” refers to the EU numbering convention for the constant regions of an antibody, as described in Edelman, G. M. et al., Proc. Natl. Acad. USA, 63, 78-85 (1969) and Kabat et al, Sequences of Proteins of Immunological Interest, U.S. Dept. Health and Human Services, 5th edition, 1991, each of which is herein incorporated by reference in its entirety.

As used herein, the term “treat,” “treating,” and “treatment” refer to therapeutic or preventative measures described herein. The methods of “treatment” employ administration of an antibody to a subject having a disease or disorder, or predisposed to having such a disease or disorder, in order to prevent, cure, delay, reduce the severity of, or ameliorate one or more symptoms of the disease or disorder or recurring disease or disorder, or in order to prolong the survival of a subject beyond that expected in the absence of such treatment.

As used herein, the term “pharmaceutically active substance” refers to a molecule or moiety that is used to achieve a beneficial outcome in a subject. Beneficial outcomes include, but are not limited to, diagnosis, prognosis, treatment, cure and prevention (prophylaxis) of diseases and/or symptoms and/or health problems.

As used herein, the term “effective amount” in the context of the administration of a therapy to a subject refers to the amount of a therapy that achieves a desired prophylactic or therapeutic effect.

As used herein, the term “subject” includes any human or non-human animal. In certain embodiments, the subject is a human or non-human mammal. In certain embodiments, the subject is a human.

As used herein with respect to an antibody, polypeptide, or polynucleotide, the term “isolated” refers to an antibody, polypeptide, or polynucleotide that is separated from one or more contaminants (e.g., polypeptides, polynucleotides, lipids, or carbohydrates, etc.) which are present in a natural source (e.g., in a mouse or a human) of the antibody, polypeptide, or polynucleotide. Moreover, an “isolated” antibody, polypeptide, or polynucleotide can be substantially free of other cellular material, or culture medium when produced by recombinant techniques, or substantially free of chemical precursors or other chemicals when chemically synthesized. For example, the language “substantially free” includes preparations of antibody, polypeptide, or polynucleotide having less than about 15%, 10%, 5%, 2%, 1%, 0.5%, or 0.1% (in particular less than about 10%) of other material, e.g., cellular material, culture medium, other nucleic acid molecules, chemical precursors, and/or other chemicals. All instances of “isolated antibodies” described herein are additionally contemplated as antibodies that may be, but need not be, isolated. All instances of “isolated polypeptides” described herein are additionally contemplated as polypeptides that may be, but need not be, isolated. All instances of “isolated polynucleotides” described herein are additionally contemplated as polynucleotides that may be, but need not be, isolated. All instances of “antibodies” described herein are additionally contemplated as antibodies that may be, but need not be, isolated. All instances of “polypeptides” described herein are additionally contemplated as polypeptides that may be, but need not be, isolated. All instances of “polynucleotides” described herein are additionally contemplated as polynucleotides that may be, but need not be, isolated.

The determination of “percent identity” between two sequences (e.g., amino acid sequences or nucleic acid sequences) can be accomplished using a mathematical algorithm. A specific, non-limiting example of a mathematical algorithm utilized for the comparison of two sequences is the algorithm of Karlin S & Altschul S F (1990) PNAS 87: 2264-2268, modified as in Karlin S & Altschul S F (1993) PNAS 90: 5873-5877, each of which is herein incorporated by reference in its entirety. Such an algorithm is incorporated into the NBLAST and XBLAST programs of Altschul S F et al., (1990) J Mol Biol 215: 403, which is herein incorporated by reference in its entirety. BLAST nucleotide searches can be performed with the NBLAST nucleotide program parameters set, e.g., for score=100, wordlength=12 to obtain nucleotide sequences homologous to a nucleic acid molecule described herein. BLAST protein searches can be performed with the XBLAST program parameters set, e.g., to score 50, wordlength=3 to obtain amino acid sequences homologous to a protein molecule described herein. To obtain gapped alignments for comparison purposes, Gapped BLAST can be utilized as described in Altschul S F et al., (1997) Nuc Acids Res 25: 3389-3402, which is herein incorporated by reference in its entirety. Alternatively, PSI BLAST can be used to perform an iterated search which detects distant relationships between molecules (Id.). When utilizing BLAST, Gapped BLAST, and PSI Blast programs, the default parameters of the respective programs (e.g., of XBLAST and NBLAST) can be used (see, e.g., National Center for Biotechnology Information (NCBI) on the worldwide web, ncbi.nlm.nih.gov). Another specific, non-limiting example of a mathematical algorithm utilized for the comparison of sequences is the algorithm of Myers and Miller, 1988, CABIOS 4: 11-17, which is herein incorporated by reference in its entirety. Such an algorithm is incorporated in the ALIGN program (version 2.0) which is part of the GCG sequence alignment software package. When utilizing the ALIGN program for comparing amino acid sequences, a PAM120 weight residue table, a gap length penalty of 12, and a gap penalty of 4 can be used.

The percent identity between two sequences can be determined using techniques similar to those described above, with or without allowing gaps. In calculating percent identity, typically only exact matches are counted.

### Anti-LY6K Antibodies

In one aspect, the instant disclosure provides antibodies that specifically bind to LY6K (e.g., human LY6K). The VH, VL, CDRH and CDRL amino acid sequences of exemplary antibodies are set forth in Tables 1-4, respectively.

TABLE 1
VH amino acid sequences of exemplary anti-LY6K antibodies.

| | | SEQ |
|---|---|---|
| Ab | VH Amino Acid Sequence | ID |
| 2659 | EVQLVESGGGLVQPGRSLRLSCAASGFTFDDYAMHWVRQAPGKGLEWVSGISWKSGI | 1 |
| | IGYADSVKGRFTISRDNAKNSLYLQMNSLRAEDTALYYCAKDSRTGLDYWGQGTLVT | |
| | VSS | |
| 2660 | EVQLVESGGGLVKPGGSLRLSCAASGFTFSDVWMSWVRQAPGKGLEWVGRIKSKTDG | 2 |
| | GRIDYAAPVKGRFTISRDDSKNTLYLQMNSLKTEDTAVYYCTTVPYSDYWGQGTLVT | |
| | VSS | |
| 3035 | EVQLVESGGGLVQPGGSLKLSCAASGFTFSDSTLHWVRQASGKGLEWVGRIRSKANS | 3 |
| | YATAYAASVKGRFTISRDDSKNTAYLQMNSLKTEDTAVYYCHNWSYGYWGQGTLVTV | |
| | SS | |
| 3047 | EVQLVESGGGLVQPGGSLRLSCAASGFTFSSYSMNWVRQAPGKGLEWVSGINWNGGS | 4 |
| | TGYADSVKGRFTISRDNAKNSLYLQMNSLRAEDTALYYCARGRLFDYWGQGTLVTVS | |
| | S | |
| 3048 | EVQLVESGGGLVQPGRSLRLSCAASGFTFDDYAMHWVRQAPGKGLEWVSGITWKSGT | 5 |
| | IGYADSVKGRFTISRDNAKNSLYLQMNSLRAEDTALYYCAKDSRTGLDYWGQGALVT | |
| | VSS | |
| 3055 | EVQLVESGGGLVQPGRSLRLSCAASGFTFDDFAMHWVRQAPGKGLEWVSGISWNTGT | 6 |
| | IGYADSVKGRFTISRDNAKNSLYLQMNSLRAEDTALYYCAKDKGTTGFDYWGQGTLV | |
| | TVSS | |
| 3056 | EVQLVESGGGLVQPGRSLRLSCAASGFTYDDYAMHWVRQVPGKGLEWVSGITWKKSI | 7 |
| | IGYADSVRGRFTVSRDSAKNSLYLQMSSLRVEDTALCYCAKDSTDGLDYWGQGTLVT | |
| | VSS | |
| 3063 | EVQLVESGGGLVKPGRSLRLSCAASGFTFDDFAMHWVRQAPGKGLEWVSGLSWKGHT | 8 |
| | LGYADSVKGRFTFSRDSAKNSLYLQMNSLRAEDTALYYCAKDTRSGLDYWGQGTLVT | |
| | VSS | |
| 3074 | EVQLVESGGGLVHPGRSLRLSCAASGFTFDDYAMHWVRQAPGKGLEWVSGISWKTNK | 9 |
| | IDYADSVKGRFTTSRDNAKNSLYLQMNSLRTEDTAFYYCAKDRREMTFDIWGQGTMV | |
| | TVSS | |
| 3075 | QVQLVQSGSELKRPGASVKVSCKASGYTFIRYAMNWVRQAPGQGLEWMGWINTNTGN | 10 |
| | PTYAQGFTGRFVLSVDTSVSTAYLQISSLKVEDTAVYYCGRPGISSFEYWGQGTLVT | |
| | VSS | |
| 3082 | EVQLVESGGGLVKPGGSLRLSCAASGFTFSSSSMNWVRQAPGKGLEWVSSISRSSYY | 11 |
| | IYYADSVEGRFTISRDNAKNSLYLQMNSLRAEDTAVYYCAKIGSSSEGVEDYYYYYY | |
| | MDVWGKGTTVTVSS | |
| 3090 | QVQLQESGPGLVKPSGTLSLTCAVSGGSISRSNWWSWVRQTPGKGLDWIGKIYHSGS | 12 |
| | TNYNPSLKSRVTISVDKSKNRFSLNLSSVTAADTAVYYCASSGSHYYYNYMEVWGKG | |
| | TTVTVSS | |
| 3096 | QVQLVQSGAEVKKPGASVRVSCKASGYTFTGYYIHWVRQAPGQGLEWMGWINPNSGG | 13 |
| | TNYAQKFQDRVTMTRDTSISTVYMELSRLRSDDTAVFFCAREASNFWGQGTLVTVSS | |
| 3101 | EVQLVESGGGLVQPGRSLRLSCAASGFTFDDYAMHWVRQTPGKGLEWVSGITWKSGN | 14 |
| | IGYADSVKGRFTISRDNAKNSLYLQMNSLRAEDTALYYCAKDTEAGLDYWGQGTLVT | |
| | VSS | |
| 3102 | QVQLVQSGAEVKKPGASVKVSCKASGYTFTGYYIHWMRQAPGQGLEWMGWINPNSGG | 15 |
| | TNYAQNFQGRVTMTRDTSINTAYMELSRLKSDDTAVYYCAKESGSPWGQGTLVTVSS | |
| 3481 | EVQLVESGGGLVQSGRSLRLSCAASGFTFDDYAMHWVRQAPGKGLEWVSGITWKDVT | 16 |
| | IGYADSVKGRFTISRDNAKNSLYLQMNSLGAEDTALYYCAKDTSDGLDYWGQGTLVT | |
| | VSS | |
| 3501 | EVQLVESGGGLVQPGRSLRLSCAASGFTFDDYAMHWVRQAPGKGLEWVSGISWNSGT | 17 |
| | IDYADSVKGRFTISRDNAKNSLYLQVNSLRAEDTALYYCAKDSPQKDYYYMDVWGKG | |
| | TTVTVSS | |
| 3503 | EVQLVESGGGLVKPGGSLRLSCAASGFIFSSYSMNWVRQAPGKGLEWVSSISSSSSY | 18 |
| | IYYADSEKGRFTISRDNAKNSLYLQMNSLRAEDTAVYYCARQSTVVTSDYYYYYMDV | |
| | WGKGTTVTVSS | |
| 3507 | EVQLVESGGGLVKPGGSLRLSCAASGFTFSSYSMNWVRQAPGKGLEWVSSISSSSDY | 19 |
| | IYYADSVKGRFTISRDDAKNSLYLQMNSLRAEDTAVYYCARGLEPNYYYYMDVWGKG | |
| | TTVTVSS | |
| 3514 | QVQLVQSGAEVKKPGASVKVSCKASGYTFTSYGISWVRQATGQGLEWMGWMNPNSGN | 20 |
| | TGYAQKFQGRVTMTRNTSISTAYMELSSLRSEDTAVYYCRAAGYYYYYYYMDVWGKG | |
| | TTVTVSS | |
| 3518 | EVQLVESGGGLVQPGGSLKLSCAASGFTFSGSAMNWVRQASGKGLEWVGRIRSKANS | 21 |
| | YATAYAASLKGRFTISRDDSKNTAYLQMNSLKTEDTAVYYCTVTPFDYWGQGTLVTV | |
| | SS | |
| 3520 | QVQLVQSGSELKKPGASVKVSCKASGYTFTLYAMNWVRQAPGQGLEWMGWINTNTGN | 22 |
| | PTYAQGFTGRFVFSLDTSVSTAYLQISSLKAEDTAVYYCARTNWNSWGQGTMVTVSS | |
| 3522 | QVQLQQSGPGLVKPSQTLSLTCAISGDSVSSNSAAWNWIRQSPSRGLEWLGRTYYRS | 23 |
| | KWYNDYAVSVKSRITINPDTSKNQFSLQLNSVTPEDTAVYYCARECSGSYYRFDPWG | |
| | QGTLVTVSS | |
| 3527 | EVQLVESGGGLVKPGGSLRLSCAASGFTFSSYSMNWVRQAPGKGLEWVSSISSTSSY | 24 |
| | IYYADSVKGRFTISRDNAKNSLYLQMNSLRAEDTAVYYCARSDYYDSGSYKNYYYYY | |
| | MDVWGKGTTVTVSS | |
| 3529 | EVQLVESGGGVVRPGGSLRLSCAASGFTFDDYGMSWVRQAPGKGLEWVSGINWNGGS | 25 |
| | TGYADSVKGRFTISRDNAKNSLYLQMNSLRAEDTALYYCARENILTGYYNLYYYYMD | |
| | VWGKGTTVTVSS | |
| 3553 | QVQLVQSGAEVKKPGASVKVSCKASGYTFTGYYMHWVRQAPGTGLDWMGWISPNSGG | 26 |
| | TNYAQKFQGRVTMTRDTSISTVYMELSRLRSDDTALYYCAREVGNYWGQGTLVTVSS | |
| 3704 | EVQLVESGGGLVQPGRSLRLSCAASGFTFDDYAMHWVRQAPGKGLEWVSGISWKIIT | 27 |
| | IGYADSVKGRFTISRDNVKNSLYLQMNSLRAEDTALYYCAKDSRTGLDYWGQGTLVT | |
| | VSS | |
| 3705 | EVQLVESGGGLVHPGRSLRLSCAASGFTFDDYAMHWVRQAPGKGLEWVSGISWKNGK | 28 |
| | IDYADSVKGRFITSRDNAKNSLYLQMNSLRTEDTALYYCAKDRIEMTFDIWGQGTMV | |
| | TVSS | |
| 3719 | EVQLVESGGGLVQPGRSLRLSCTASGFTFNDYAMHWVRQAPGKGLEWVSGISWNSNN | 29 |
| | IDYADSVKGRFTISRDNAKNSLYMQMNSLRAEDTALYYCAKDGPGTTNDAFDIWGQG | |
| | TMVTVSS | |
| 3724 | QLQLQESGPGLVKPSETLSLTCTVSGGSINSKSYYWDWIRQPPGKGLEWIGNIYYSG | 30 |
| | STYYNPSLKSRVTISVDTSKNQFSLKLSSVTATDTAVYYCARHRGVTVTTYWYFDLW | |
| | GRGTLVTVSS | |
| 3728 | EVQLVESGGGLVKPGGSLRLSCAASGFTFSDVWMSWVRQAPGKGLEWVGRIKSKTDG | 2 |
| | GRIDYAAPVKGRFTISRDDSKNTLYLQMNSLKTEDTAVYYCTTVPYSDYWGQGTLVT | |
| | VSS | |
| 3741 | EVQLVESGGGLVKPGGSLRLSCAASGFTFSSYSMNWVRQAPGKGLEWVSSISSSSNY | 31 |
| | IYYVDSVKGRFTTSRDNAKNSLYLQMNSLRAEDTAVYYCARQEATGNNYYYYYMDVW | |
| | GKGTTVTVSS | |
| 3743 | EVQLVESGGGLVHPGRSLRLSCAASGFTFDDYAMHWVRQAPGKGLEWVSGISWKSGR | 32 |
| | IDYADSVKGRFTISRDNAKNSLYLQMNSLRAEDTALYYCAKDRRELTFDIWGQGTMV | |
| | TVSS | |
| 3746 | EVQLVESGGGLVKPGGSLRLSCAASGFIFSSYSMNWVRQAPGKGLEWVSSISSSSSY | 18 |
| | IYYADSEKGRFTISRDNAKNSLYLQMNSLRAEDTAVYYCARQSTVVTSDYYYYYMDV | |
| | WGKGTTVTVSS | |
| 3785 | QVQLQQSGPGLVKPSQTLSLTCAISGDSVSSNSAAWNWIRQSPSRGLEWLGRTYFRS | 33 |
| | KWNNDYTLSVKNRITINPDTSKNHFSLQLNSVTPEDTAVYYCAGGGYCNRTYCPDVF | |
| | DIWGQGTMVTVSS | |
| 3815 | EVQLVESGGGVVRPGGSLRLSCAASGFTFDDYGMSWVRQAPGKGLEWVSGINWNGGS | 34 |
| | TGYVDSVKGRFTISRDNAKNSLYLQMNSLRAEDTALYYCARMFYGAFDIWGQGTMVT | |
| | VSS | |

TABLE 2
VL amino acid sequences of exemplary anti-LY6K antibodies.

| | | SEQ |
|---|---|---|
| Ab | VL Amino Acid Sequence | ID |
| 2659 | DIVMTQTPLSSPVTLGQPASISCRSSQSLVHSDGNTYLSWLQQRPGQPPRLLIYKIS | 35 |
| | NRFSGVPDRFSGSGAGTDFTLKISRVEAEDVGVYYCMQATQFPYTFGQGTKLEIK | |
| 2660 | DIVMTQSPDSLAVSLGERATINCKSSQSVLYSSDNENYLAWYQQKPGQPPKLLIYWA | 36 |
| | SSRESGVPDRFSGSGSGTDFTLTISSLQAEDVAVYYCHQYYNTPWTFGQGTKVEIK | |
| 3035 | QTVVTQEPSLTVSPGGTVTLTCASSTGAVTSYYYPNWFQQKPGQAPRTVIYSTTNKH | 37 |
| | SWTPARFSGSLLGGKAALTLSGVLPEDEADYYCLLFYGGAYVFGTGTKVTVL | |
| 3047 | DIVMTQSPDSLAVSLGERATINCKSSQSVLYSSNNKNYLAWYQQKPGQPPKLLIYWA | 38 |
| | STRESGVPDRFSGSGSGTDFTLTISSLQAEDVAVYYCQQYYSTPSYTFGQGTKLEIK | |
| 3048 | DIVMTQSPLSSPVTLGQPASISCRSSQSLVHSDGNTYLSWLQQRPGQPPRLLIYKIS | 39 |
| | NRFSGVPDRFSGSGAGTDFTLKISRVEAEDVGVYYCMQATQFPYTFGQGTRLEIK | |
| 3055 | DIQMTQSPSSVSASVGDRVTITCRASQGISRWLVWYQQKPGKAPKLLIYAASSLQSG | 40 |
| | VPSRFSGSGSGTDFTLTISSLQPEDFATYYCQQANSFPRTFGQGTRLEIK | |
| 3056 | DIVMTQTPLSSPVTLGQPASLSCRSSQSLLHSNGNTYLSWLQQRPGQPPRLLIYKIS | 41 |
| | NRFSGVPDRFSGSGAGTDFTLKISRVEGEDVGVYYCMQATEFPYTFGQGTKLEIK | |
| 3063 | DIVMTQTPLSSPVTLGQPASISCRSSQSLEHSDGNTYLSWLQQRPGQPPRLLIYKIS | 42 |
| | NRFSGVPDRFSGNGAGTDFTLKISRVEAEDVGIYYCMQATEFPFTFGGGTKVEIK | |
| 3074 | DIVMTQTPLSSPVTLGQPASISCRSSQSLEHSNGNTYLSWLQQRPGQPPRLLIYKIS | 43 |
| | NRFSGVPDRFSGSGAGTDFTLKISRVEAEDVGIYYCMQATQFPLTFGGGTKVEIK | |
| 3075 | DIQMTQSPSSLSASVGDRVAITCRATQDISNYLDWYQQKPGKAPKLLIYDASNLETG | 44 |
| | VPSRFSGSGSGTDFTFTINSLQPEDIATYYCQQYDNLPLTFGGGTKVEIK | |
| 3082 | DIQMTQSPSSLSASVGDRVTITCRASQNINSYLNWYQQKPGKAPKLLIYAASSLQSG | 45 |
| | VPSRFSGSGSGTDFTLTISSLQPEDFATYYCHQSYSTPLTFGGGTKVEIK | |
| 3090 | EIVLTQSPGTLSLSPGERATLSCRASQTVSSSYLVWYQQKPGQAPRLLIYGASGRAT | 46 |
| | GIPDRFSGSGSGTDFTLTISRLEPEDFAVFYCQHYGSSPYTFGQGTKLEIK | |
| 3096 | DIQMTQSPSSLSASIGDRVTITCQASQDISNYLNWYQQKPGKAPKLLIYDASNLETG | 47 |
| | VPSRFSGSGSGTDFTFTISSLQPEDIATYYCQQYESLPFNFGGGTKVEIK | |
| 3101 | DIVMTQTPLSSPVTLGQPASISCRSSQSLVHSNGNTYLSWLQQRPGQPPRLLIYKIS | 48 |
| | NRFSGVPDRFSGSGAGTDFTLKISRVEAEDVGVYYCMQATQFPYTFGQGTKLEIK | |
| 3102 | DIQMTQSPSSLSASVGDRVTITCQASQDISNYLNWYQQKPGKAPKLLIYDASNLERG | 49 |
| | VPSRFSGSGSGTDFTFSISSLQPEDIATYHCQEYDSLPFTFGPGTKVDIK | |
| 3481 | DIVMTQTPLSSPVTLGQPASISCRSSQSLVHSNGNTYLSWLQQRPGQPPRLLIYKIS | 50 |
| | NRFSGVPDRFSGSGAGTDFTLKISRVETEDVGIYYCMQAIQFPYTFGQGTKLEIK | |
| 3501 | DIQMTQSPSSLSASVGDRVTITCRTSQSISSYLNWYQQKPGKAPNLLIYAASSLQSG | 51 |
| | VPSRFSGSGSGTDFTLTISSLQPEDFATYYCQQSYITPITFGQGTRLEIK | |
| 3503 | DIQMTQSPSSLSASVGDRVTITCRASQSISNYLNWYQQKPGKAPNLLIYAASSLQSG | 52 |
| | VPSRFSGSGSGTDFTLTISSLQPEDFATYYCQQSYSPPITFGQGTRLEIK | |
| 3507 | EIVMTQSPGTLSVSPGERATLSCRASQSVSSSYLAWYQQKPGQAPRLLIYGASTRAT | 53 |
| | GIPARFSGSGSGTEFTLTISSLQSEDFAVYYCQQYNNWPLTFGGGTKVEIK | |
| 3514 | DIVMTQSPLSLPVTPGEPASISCRSSQSLLHSNGYNYLDWYLQKPGQSPQLLIYLGS | 54 |
| | NRASGVPDRFSGSGSGTDFTLKISRVEAEDVGVYYCMQALQTYTFGQGTKLEIK | |
| 3518 | DIQMTQSPSSLSASVGDRVTITCQASQDISNHLNWYQQKPGKAPKLLINDASNLETG | 55 |
| | VPSRFSGSGSGTDFTFTISSLQPEDIATYYCQQYDNVPYTFGQGTKLEIK | |
| 3520 | DIQMTQSPSSLSASVGDRVTITCQASQDISNYLHWFQQKPGKAPKLLIYDASNLETG | 56 |
| | VPSRFSGSGSGTDFTFTIFSLQPEDIATYYCQHYDNLPLTFGGGTKVEIK | |
| 3522 | DIVMTQTPLSSPVTLGQPASISCRSSQSLVHSNGNTYLSWLQQRPGQPPRLLIYKIS | 57 |
| | HRFSGVPDRFSGSGAGTDFTLKIRRVEAEDVGVYYCMQATQFPYTFGQGTKLEIK | |
| 3527 | DIQMTQSPSSLSASVGDRVTITCRTSQSISSYLNWFQQKPGKAPKVLIYAASSLQRG | 58 |
| | VPSRFSGSGSGTDFTLTISSLQPEDFATYYCQQSYSTPLTFGGGTKVEIK | |
| 3529 | DIQMTQSPSSLSASVGDRVTITCRASQSISSYLNWYQQKPGKAPKLLIYAASSLQSG | 59 |
| | VPSRFSGSGSGTDFTLTISSLQPEDFATYYCQQSYSTSHGGGTKVEIK | |
| 3553 | DIQMTQSPTSLSTSVGDRVTITCQASQDIYNYLNWYQLKPGKAPKLLIYDASNLERG | 60 |
| | VTSRFSGSGSGTDFTFTISSLQPEDIATYYCQQYDSLPITFGQGTRLEIK | |
| 3704 | DIQMTQSPSSLSASVGDRVTITCRASQTISSYLNWYQQKPGKAPKLLIYAASSLQSG | 61 |
| | VPSRFSGSGSGTDFTLTISSLQPEDFATYYCQQTYTAPLTFGPGTKVDIK | |
| 3705 | DIVMTQSPLSSPVTLGQPASISCRSSQSLEHSNGNTYLSWLQQRPGQPPRLLIYKIS | 62 |
| | NRFSGVPDRFSGSGAGTDFTLKISRVEAEDVGVYYCMQTTQFPLTFGGGTKVEIK | |
| 3719 | DIQMTQSPSSLSASVGDRVTITCRASHSISSYLNWYQQKPGKAPKLLIYAASSLQSG | 63 |
| | VPSRFSGSGSGTDFTLTISSLQPEDFATYYCHQGYSTPLTFGGGTKVEIK | |
| 3724 | EIVLTQSPDFQSVTPKEKVTITCRASQSIGNSLHWYQQKSDQSPKLLIKYVSQSFSG | 64 |
| | VPSRFSGSGSGTDFTLTINSLEAEDAATYYCHQSSDFPHTFGQGTKLEIK | |
| 3728 | DIVMTQSPDSLAVSLGERATINCKSSQSVLYSSDNENYLAWYQQKPGQPPKLLIYWA | 36 |
| | SSRESGVPDRFSGSGSGTDFTLTISSLQAEDVAVYYCHQYYNTPWTFGQGTKVEIK | |
| 3741 | DIQMTQSPSSLSASVGDRVTITCRASQSISNYLNWYQQNLGKAPKVLIYAASSLQSG | 65 |
| | VPSRFSGSGSGTDFTLTISSLQPEDFATYYCQQTYSTPITFGQGTRLEIK | |
| 3743 | DIVMTQTPLSSPVTLGQPASISCRSSQSLEHSNGNTYLSWLQQRPGQPPRLLIYKIS | 66 |
| | NRFSGVPDRFSGSGAGTDFTLKISRVEAEDVGVYYCMQTTQFPLTFGGGTKVEIK | |
| 3746 | DIQMTQSPSSLSASVGDRVTITCRASQSISNYLNWYQQKPGKAPNLLIYAASSLQSG | 52 |
| | VPSRFSGSGSGTDFTLTISSLQPEDFATYYCQQSYSPPITFGQGTRLEIK | |
| 3785 | DIQMTQSPSSLSASVGDRVTFTCRSSQSISSYLNWYQQKPGKAPNLLIYVASTLQSG | 67 |
| | VPSRFSGSGSGTDFTLTISSLQPEDFATYYCQQSYSIPLTFGGGTKVEIK | |
| 3815 | EIVLTQSPGTLSLSPGERATLSCRASQSVSSNYLAWYQQKPGQAPRLLIYGASSRAT | 68 |
| | GIPDRFSGSGSGTDFTLTISRLEPEDFAVYYCQQYGSSPFTFGQGTKVDIK | |

TABLE 3
CDRH amino acid sequences of exemplary anti-LY6K antibodies.

| | CDRH1 amino | SEQ | CDRH2 amino | SEQ | CDRH3 amino | SEQ |
|---|---|---|---|---|---|---|
| Ab | acid sequence | ID | acid sequence | ID | acid sequence | ID |
| 2659 | GFTFDDYA | 69 | ISWKSGII | 90 | AKDSRTGLDY | 124 |
| 2660 | GFTFSDVW | 70 | IKSKTDGGRI | 91 | TTVPYSDY | 125 |
| 3035 | GFTFSDST | 71 | IRSKANSYAT | 92 | HNWSYGY | 126 |
| 3047 | GFTFSSYS | 72 | INWNGGST | 93 | ARGRLFDY | 127 |
| 3048 | GFTFDDYA | 69 | ITWKSGTI | 94 | AKDSRTGLDY | 124 |
| 3055 | GFTFDDFA | 73 | ISWNTGTI | 95 | AKDKGTTGFDY | 128 |
| 3056 | DYAMH | 74 | GITWKKSIIGYADSVRG | 96 | DSTDGLDY | 129 |
| 3063 | DFAMH | 75 | GLSWKGHTLGYADSVKG | 97 | DTRSGLDY | 130 |
| 3074 | DYAMH | 74 | GISWKTNKIDYADSVKG | 98 | DRREMTFDI | 131 |
| 3075 | RYAMN | 76 | WINTNTGNPTYAQGFTG | 99 | PGISSFEY | 132 |
| 3082 | SSSMN | 77 | SISRSSYYIYYADSVEG | 100 | IGSSSEGVEDYYYY | 133 |
| | | | | | YYMDV | |
| 3090 | RSNWWS | 78 | KIYHSGSTNYNPSLKS | 101 | SGSHYYYNYMEV | 134 |
| 3096 | GYYIH | 79 | WINPNSGGTNYAQKFQD | 102 | EASNF | 135 |
| 3101 | DYAMH | 74 | GITWKSGNIGYADSVKG | 103 | DTEAGLDY | 136 |
| 3102 | GYYIH | 80 | WINPNSGGTNYAQNFQG | 104 | ESGSP | 137 |
| 3481 | DYAMH | 74 | GITWKDVTIGYADSVKG | 105 | DTSDGLDY | 138 |
| 3501 | DYAMH | 74 | GISWNSGTIDYADSVKG | 106 | DSPQKDYYYMDV | 139 |
| 3503 | SYSMN | 81 | SISSSSSYIYYADSEKG | 107 | QSTVVTSDYYYYYM | 140 |
| | | | | | DV | |
| 3507 | SYSMN | 81 | SISSSSDYIYYADSVKG | 108 | GLEPNYYYYMDV | 141 |
| 3514 | SYGIS | 82 | WMNPNSGNTGYAQKFQG | 109 | AGYYYYYYYMDV | 142 |
| 3518 | GSAMN | 83 | RIRSKANSYATAYAASLKG | 110 | TPFDY | 143 |
| 3520 | LYAMN | 84 | WINTNTGNPTYAQGFTG | 99 | TNWNS | 144 |
| 3522 | SNSAAWN | 85 | RTYYRSKWYNDYAVSVKS | 111 | ECSGSYYRFDP | 145 |
| 3527 | SYSMN | 81 | SISSTSSYIYYADSVKG | 112 | SDYYDSGSYKNYYY | 146 |
| | | | | | YYMDV | |
| 3529 | DYGMS | 86 | GINWNGGSTGYADSVKG | 113 | ENILTGYYNLYYYY | 147 |
| | | | | | MDV | |
| 3553 | GYYMH | 87 | WISPNSGGTNYAQKFQG | 114 | EVGNY | 148 |
| 3704 | DYAMH | 74 | GISWKIITIGYADSVKG | 115 | DSRTGLDY | 149 |
| 3705 | DYAMH | 74 | GISWKNGKIDYADSVKG | 116 | DRIEMTFDI | 150 |
| 3719 | DYAMH | 74 | GISWNSNNIDYADSVKG | 117 | DGPGTTNDAFDI | 151 |
| 3724 | SKSYYWD | 88 | NIYYSGSTYYNPSLKS | 118 | HRGVTVTTYWYFDL | 152 |
| 3728 | DVWMS | 89 | RIKSKTDGGRIDYAAPVKG | 119 | VPYSDY | 153 |
| 3741 | SYSMN | 81 | SISSSSNYIYYVDSVKG | 120 | QEATGNNYYYYYMD | 154 |
| | | | | | V | |
| 3743 | DYAMH | 74 | GISWKSGRIDYADSVKG | 121 | DRRELTFDI | 155 |
| 3746 | SYSMN | 81 | SISSSSSYIYYADSEKG | 107 | QSTVVTSDYYYYYM | 140 |
| | | | | | DV | |
| 3785 | SNSAAWN | 85 | RTYFRSKWNNDYTLSVKN | 122 | GGYCNRTYCPDVFD | 156 |
| | | | | | I | |
| 3815 | DYGMS | 86 | GINWNGGSTGYVDSVKG | 123 | MFYGAFDI | 157 |

TABLE 4
CDRL amino acid sequences of exemplary anti-LY6K antibodies.

| | CDRL1 amino | SEQ | CDRL2 amino | SEQ | CDRL3 amino | SEQ |
|---|---|---|---|---|---|---|
| Ab | acid sequence | ID | acid sequence | ID | acid sequence | ID |
| 2659 | QSLVHSDGNTY | 158 | KIS | — | MQATQFPYT | 198 |
| 2660 | QSVLYSSDNENY | 159 | WAS | — | HQYYNTPWT | 199 |
| 3035 | TGAVTSYYY | 160 | STT | — | LLFYGGAYV | 200 |
| 3047 | QSVLYSSNNKNY | 161 | WAS | — | QQYYSTPSYT | 201 |
| 3048 | QSLVHSDGNTY | 158 | KIS | — | MQATQFPYT | 198 |
| 3055 | QGISRW | 162 | AAS | — | QQANSFPRT | 202 |
| 3056 | RSSQSLLHSNGNTYLS | 163 | KISNRFS | 185 | MQATEFPYT | 203 |
| 3063 | RSSQSLEHSDGNTYLS | 164 | KISNRFS | 185 | MQATEFPFT | 204 |
| 3074 | RSSQSLEHSNGNTYLS | 165 | KISNRFS | 185 | MQATQFPLT | 205 |
| 3075 | RATQDISNYLD | 166 | DASNLET | 186 | QQYDNLPLT | 206 |
| 3082 | RASQNINSYLN | 167 | AASSLQS | 187 | HQSYSTPLT | 207 |
| 3090 | RASQTVSSSYLV | 168 | GASGRAT | 188 | QHYGSSPYT | 208 |
| 3096 | QASQDISNYLN | 169 | DASNLET | 186 | QQYESLPFN | 209 |
| 3101 | RSSQSLVHSNGNTYLS | 170 | KISNRFS | 185 | MQATQFPYT | 198 |
| 3102 | QASQDISNYLN | 169 | DASNLER | 189 | QEYDSLPFT | 210 |
| 3481 | RSSQSLVHSNGNTYLS | 170 | KISNRFS | 185 | MQAIQFPYT | 211 |
| 3501 | RTSQSISSYLN | 171 | AASSLQS | 187 | QQSYITPIT | 212 |
| 3503 | RASQSISNYLN | 172 | AASSLQS | 187 | QQSYSPPIT | 213 |
| 3507 | RASQSVSSSYLA | 173 | GASTRAT | 190 | QQYNNWPLT | 214 |
| 3514 | RSSQSLLHSNGYNYLD | 174 | LGSNRAS | 191 | MQALQTYT | 215 |
| 3518 | QASQDISNHLN | 175 | DASNLET | 186 | QQYDNVPYT | 216 |
| 3520 | QASQDISNYLH | 176 | DASNLET | 186 | QHYDNLPLT | 217 |
| 3522 | RSSQSLVHSNGNTYLS | 170 | KISHRFS | 192 | MQATQFPYT | 198 |
| 3527 | RTSQSISSYLN | 171 | AASSLQR | 193 | QQSYSTPLT | 218 |
| 3529 | RASQSISSYLN | 177 | AASSLQS | 187 | QQSYSTS | 219 |
| 3553 | QASQDIYNYLN | 178 | DASNLER | 189 | QQYDSLPIT | 220 |
| 3704 | RASQTISSYLN | 179 | AASSLQS | 187 | QQTYTAPLT | 221 |
| 3705 | RSSQSLEHSNGNTYLS | 165 | KISNRFS | 185 | MQTTQFPLT | 222 |
| 3719 | RASHSISSYLN | 180 | AASSLQS | 187 | HQGYSTPLT | 223 |
| 3724 | RASQSIGNSLH | 181 | YVSQSFS | 194 | HQSSDFPHT | 224 |
| 3728 | KSSQSVLYSSDNENYLA | 182 | WASSRES | 195 | HQYYNTPWT | 199 |
| 3741 | RASQSISNYLN | 172 | AASSLQS | 187 | QQTYSTPIT | 225 |
| 3743 | RSSQSLEHSNGNTYLS | 165 | KISNRFS | 185 | MQTTQFPLT | 222 |
| 3746 | RASQSISNYLN | 172 | AASSLQS | 187 | QQSYSPPIT | 213 |
| 3785 | RSSQSISSYLN | 183 | VASTLQS | 196 | QQSYSIPLT | 226 |
| 3815 | RASQSVSSNYLA | 184 | GASSRAT | 197 | QQYGSSPFT | 227 |

The individual CDRs of an antibody disclosed herein can be determined according to any CDR numbering scheme known in the art.

In certain embodiments, one or more of the CDRs of an antibody disclosed herein can be determined according to Kabat et al., J. Biol. Chem. 252, 6609-6616 (1977) and Kabat et al., Sequences of protein of immunological interest (1991), each of which is herein incorporated by reference in its entirety.

In certain embodiments, an antibody provided herein comprises the CDRH1, CDRH2, and/or CDRH3 of a VH amino acid sequence set forth in any of SEQ ID NOs: 1-34 as determined by the Kabat numbering scheme. In certain embodiments, an antibody provided herein comprises the CDRL1, CDRL2, and/or CDRL3 of a VL amino acid sequence set forth in any of SEQ ID NOs: 35-68 as determined by the Kabat numbering scheme.

In certain embodiments, one or more of the CDRs of an antibody disclosed herein can be determined according to the Chothia numbering scheme, which refers to the location of immunoglobulin structural loops (see, e.g., Chothia C & Lesk A M, (1987), J Mol Biol 196: 901-917; Al-Lazikani B et al., (1997) J Mol Biol 273: 927-948; Chothia C et al., (1992) J Mol Biol 227: 799-817; Tramontano A et al., (1990) J Mol Biol 215(1): 175-82; and U.S. Pat. No. 7,709,226, all of which are herein incorporated by reference in their entireties).

In certain embodiments, an antibody provided herein comprises the CDRH1, CDRH2, and/or CDRH3 of a VH amino acid sequence set forth in any of SEQ ID NOs: 1-34 as determined by the Chothia numbering system. In certain embodiments, an antibody provided herein comprises the CDRL1, CDRL2, and/or CDRL3 of a VL amino acid sequence set forth in any of SEQ ID NOs: 35-68 as determined by the Chothia numbering system.

In certain embodiments, one or more of the CDRs of an antibody disclosed herein can be determined according to MacCallum R M et al., (1996) J Mol Biol 262: 732-745, herein incorporated by reference in its entirety. See also, e.g., Martin A. “Protein Sequence and Structure Analysis of Antibody Variable Domains,” in Antibody Engineering, Kontermann and Dubel, eds., Chapter 31, pp. 422-439, Springer-Verlag, Berlin (2001), herein incorporated by reference in its entirety.

In certain embodiments, an antibody provided herein comprises the CDRH1, CDRH2, and/or CDRH3 of a VH amino acid sequence set forth in any of SEQ ID NOs: 1-34 as determined by the MacCallum numbering system. In certain embodiments, an antibody provided herein comprises the CDRL1, CDRL2, and/or CDRL3 of a VL amino acid sequence set forth in any of SEQ ID NOs: 35-68 as determined by the MacCallum numbering system.

In certain embodiments, the CDRs of an antibody disclosed herein can be determined according to the IMGT numbering system as described in: Lefranc M-P, (1999) The Immunologist 7: 132-136; Lefranc M-P et al., (1999) Nucleic Acids Res 27: 209-212, each of which is herein incorporated by reference in its entirety; and Lefranc M-P et al., (2009) Nucleic Acids Res 37: D1006-D1012.

In certain embodiments, an antibody provided herein comprises the CDRH1, CDRH2, and/or CDRH3 of a VH amino acid sequence set forth in any of SEQ ID NOs: 1-34 as determined by the IMGT numbering system. In certain embodiments, an antibody provided herein comprises the CDRL1, CDRL2, and/or CDRL3 of a VL amino acid sequence set forth in any of SEQ ID NOs: 35-68 as determined by the IMGT numbering system.

In certain embodiments, the CDRs of an antibody disclosed herein can be determined according to the AbM numbering scheme, which refers to AbM hypervariable regions, which represent a compromise between the Kabat CDRs and Chothia structural loops, and are used by Oxford Molecular's AbM antibody modeling software (Oxford Molecular Group, Inc.), herein incorporated by reference in its entirety.

In certain embodiments, an antibody provided herein comprises the CDRH1, CDRH2, and/or CDRH3 of a VH amino acid sequence set forth in any of SEQ ID NOs: 1-34 as determined by the AbM numbering scheme. In certain embodiments, an antibody provided herein comprises the CDRL1, CDRL2, and/or CDRL3 of a VL amino acid sequence set forth in any of SEQ ID NOs: 35-68 as determined by the AbM numbering scheme.

In certain embodiments, the CDRs of an antibody disclosed herein can be determined according to the AHo numbering system, as described in Honegger and Pluckthun, A., J. Mol. Biol. 309:657-670 (2001), herein incorporated by reference in its entirety.

In certain embodiments, an antibody provided herein comprises the CDRH1, CDRH2, and/or CDRH3 of a VH amino acid sequence set forth in any of SEQ ID NOs: 1-34 as determined by the AHo numbering system. In certain embodiments, an antibody provided herein comprises the CDRL1, CDRL2, and/or CDRL3 of a VL amino acid sequence set forth in any of SEQ ID NOs: 35-68 as determined by the AHo numbering system.

In certain embodiments, the individual CDRs of an antibody disclosed herein are each independently determined according to one of the Kabat, Chothia, MacCallum, IMGT, AHo, or AbM numbering schemes, or by structural analysis of the multispecific molecule, wherein the structural analysis identifies residues in the variable region(s) predicted to make contact with an epitope region of LY6K.

In certain embodiments, the instant disclosure provides an antibody that specifically binds LY6K (e.g., human LY6K) comprising a VH comprising the CDRH1, CDRH2, and CDRH3 amino acid sequences of a VH amino acid sequence set forth in any one of SEQ ID NOs: 1-34, and a VL comprising the CDRL1, CDRL2, and CDRL3 amino acid sequences of a VL amino acid sequence set forth in any one of SEQ ID NOs: 35-68, wherein each CDR is independently determined according to one of the Kabat, Chothia, MacCallum, IMGT, AHo, or AbM numbering schemes, or by structural analysis of the multispecific molecule, wherein the structural analysis identifies residues in the variable region(s) predicted to make contact with an epitope region of LY6K (e.g., human LY6K).

In certain embodiments, the instant disclosure provides an antibody that specifically binds LY6K (e.g., human LY6K) comprising a VH comprising the CDRH1, CDRH2, and CDRH3 amino acid sequences of a VH amino acid sequence set forth in any one of SEQ ID NOs: 1-34, and a VL comprising the CDRL1, CDRL2, and CDRL3 amino acid sequences of a VL amino acid sequence set forth in any one of SEQ ID NOs: 35-68, wherein the VH amino acid sequence and the VL amino acid sequence are from the same antibody (i.e., as shown in Tables 1 and 2), and wherein each CDR is independently determined according to one of the Kabat, Chothia, MacCallum, IMGT, AHo, or AbM numbering schemes, or by structural analysis of the multispecific molecule, wherein the structural analysis identifies residues in the variable region(s) predicted to make contact with an epitope region of LY6K (e.g., human LY6K).

In certain embodiments, the instant disclosure provides an antibody that specifically binds to LY6K (e.g., human LY6K), wherein the antibody comprises the CDRH1, CDRH2, CDRH3, CDRL1, CDRL2, and CDRL3 amino acid sequences of the VH and VL amino acid sequences set forth in SEQ ID NOs: 1 and 35; SEQ ID NOs: 2 and 36; SEQ ID NOs: 3 and 37; SEQ ID NOs: 4 and 38; SEQ ID NOs: 5 and 39; SEQ ID NOs: 6 and 40; SEQ ID NOs: 7 and 41; SEQ ID NOs: 8 and 42; SEQ ID NOs: 9 and 43; SEQ ID NOs: 10 and 44; SEQ ID NOs: 11 and 45; SEQ ID NOs: 12 and 46; SEQ ID NOs: 13 and 47; SEQ ID NOs: 14 and 48; SEQ ID NOs: 15 and 49; SEQ ID NOs: 16 and 50; SEQ ID NOs: 17 and 51; SEQ ID NOs: 18 and 52; SEQ ID NOs: 19 and 53; SEQ ID NOs: 20 and 54; SEQ ID NOs: 21 and 55; SEQ ID NOs: 22 and 56; SEQ ID NOs: 23 and 57; SEQ ID NOs: 24 and 58; SEQ ID NOs: 25 and 59; SEQ ID NOs: 26 and 60; SEQ ID NOs: 27 and 61; SEQ ID NOs: 28 and 62; SEQ ID NOs: 29 and 63; SEQ ID NOs: 30 and 64; SEQ ID NOs: 31 and 65; SEQ ID NOs: 32 and 66; SEQ ID NOs: 33 and 67; or SEQ ID NOs: 34 and 68, respectively.

In certain embodiments, the instant disclosure provides an antibody that specifically binds to LY6K (e.g., human LY6K), wherein the antibody comprises a VH comprising a CDRH1, CDRH2, and/or CDRH3 amino acid sequence set forth in Table 3. In certain embodiments, the antibody comprises a VH comprising a CDRH1 amino acid sequence set forth in any one of SEQ ID NOs: 69-89. In certain embodiments, the antibody comprises a VH comprising a CDRH2 amino acid sequence set forth in any one of SEQ ID NOs: 90-123. In certain embodiments, the antibody comprises a VH comprising a CDRH3 amino acid sequence set forth in any one of SEQ ID NOs: 124-157.

In certain embodiments, the instant disclosure provides an antibody that specifically binds to LY6K (e.g., human LY6K), wherein the antibody comprises a VH comprising the CDRH1, CDRH2, and CDRH3 amino acid sequences of any of the antibodies in Table 3. In certain embodiments, the antibody comprises a VH comprising the CDRH1, CDRH2, and CDRH3 amino acid sequences set forth in SEQ ID NOs: 69, 90, and 124; SEQ ID NOs: 70, 91, and 125; SEQ ID NOs: 71, 92, and 126; SEQ ID NOs: 72, 93, and 127; SEQ ID NOs: 69, 94, and 124; SEQ ID NOs: 73, 95, and 128; SEQ ID NOs: 74, 96, and 129; SEQ ID NOs: 75, 97, and 130; SEQ ID NOs: 74, 98, and 131; SEQ ID NOs: 76, 99, and 132; SEQ ID NOs: 77, 100, and 133; SEQ ID NOs: 78, 101, and 134; SEQ ID NOs: 79, 102, and 135; SEQ ID NOs: 74, 103, and 136; SEQ ID NOs: 80, 104, and 137; SEQ ID NOs: 74, 105, and 138; SEQ ID NOs: 74, 106, and 139; SEQ ID NOs: 81, 107, and 140; SEQ ID NOs: 81, 108, and 141; SEQ ID NOs: 82, 109, and 142; SEQ ID NOs: 83, 110, and 143; SEQ ID NOs: 84, 99, and 144; SEQ ID NOs: 85, 111, and 145; SEQ ID NOs: 81, 112, and 146; SEQ ID NOs: 86, 113, and 147; SEQ ID NOs: 87, 114, and 148; SEQ ID NOs: 74, 115, and 149; SEQ ID NOs: 74, 116, and 150; SEQ ID NOs: 74, 117, and 151; SEQ ID NOs: 88, 118, and 152; SEQ ID NOs: 89, 119, and 153; SEQ ID NOs: 81, 120, and 154; SEQ ID NOs: 74, 121, and 155; SEQ ID NOs: 85, 122, and 156; or SEQ ID NOs: 86, 123, and 157; respectively.

In certain embodiments, the instant disclosure provides an antibody that specifically binds to LY6K (e.g., human LY6K), wherein the antibody comprises a VL comprising a CDRL1, CDRL2, and/or CDRL3 amino acid sequence set forth in Table 4. In certain embodiments, the antibody comprises a VL comprising a CDRL1 amino acid sequence set forth in any one of SEQ ID NOs: 158-184. In certain embodiments, the antibody comprises a VL comprising a CDRL2 amino acid sequence set forth in any one of KIS, WAS, STT, AAS, and SEQ ID NOs: 185-197. In certain embodiments, the antibody comprises a VL comprising a CDRL3 amino acid sequence set forth in any one of SEQ ID NOs: 198-227.

In certain embodiments, the instant disclosure provides an antibody that specifically binds to LY6K (e.g., human LY6K), wherein the antibody comprises a VL comprising the CDRL1, CDRL2, and CDRL3 amino acid sequences of any of the antibodies in Table 4. In certain embodiments, the antibody comprises a VL comprising the CDRL1, CDRL2, and CDRL3 amino acid sequences set forth in SEQ ID NO: 158, KIS, and SEQ ID NO: 198; SEQ ID NO: 159, WAS, and SEQ ID NO: 199; SEQ ID NO: 160, STT, and SEQ ID NO: 200; SEQ ID NO: 161, WAS, and SEQ ID NO: 201; SEQ ID NO: 162, AAS, and SEQ ID NO: 202; SEQ ID NOs: 163, 185, and 203; SEQ ID NOs: 164, 185, and 204; SEQ ID NOs: 165, 185, and 205; SEQ ID NOs: 166, 186, and 206; SEQ ID NOs: 167, 187, and 207; SEQ ID NOs: 168, 188, and 208; SEQ ID NOs: 169, 186, and 209; SEQ ID NOs: 170, 185, and 198; SEQ ID NOs: 169, 189, and 210; SEQ ID NOs: 170, 185, and 211; SEQ ID NOs: 171, 187, and 212; SEQ ID NOs: 172, 187, and 213; SEQ ID NOs: 173, 190, and 214; SEQ ID NOs: 174, 191, and 215; SEQ ID NOs: 175, 186, and 216; SEQ ID NOs: 176, 186, and 217; SEQ ID NOs: 170, 192, and 198; SEQ ID NOs: 171, 193, and 218; SEQ ID NOs: 177, 187, and 219; SEQ ID NOs: 178, 189, and 220; SEQ ID NOs: 179, 187, and 221; SEQ ID NOs: 165, 185, and 222; SEQ ID NOs: 180, 187, and 223; SEQ ID NOs: 181, 194, and 224; SEQ ID NOs: 182, 195, and 199; SEQ ID NOs: 172, 187, and 225; SEQ ID NOs: 183, 196, and 226; or SEQ ID NOs: 184, 197, and 227, respectively.

In certain embodiments, the instant disclosure provides an antibody that specifically binds to LY6K (e.g., human LY6K), wherein the antibody comprises a VH comprising a CDRH1, CDRH2, and/or CDRH3 amino acid sequence set forth in Table 3 and a VL comprising a CDRL1, CDRL2, and/or CDRL3 amino acid sequence set forth in Table 4, wherein 2, 3, 4, 5, or 6 of the CDRH1, CDRH2, CDRH3, CDRL1, CDRL2, and CDRL3 amino acid sequences are from the same antibody (i.e., as shown in Tables 3 and 4). In certain embodiments, the antibody comprises a VH comprising CDRH1, CDRH2, and CDRH3 regions, and a VL comprising CDRL1, CDRL2, and CDRL3 regions, wherein the CDRH1, CDRH2, CDRH3, CDRL1, CDRL2, and CDRL3 regions comprise the amino acid sequences set forth in SEQ ID NOs: 69, 90, 124, 158, KIS, and SEQ ID NO: 198; SEQ ID NOs: 70, 91, 125, 159, WAS, and SEQ ID NO: 199; SEQ ID NOs: 71, 92, 126, 160, STT, and SEQ ID NO: 200; SEQ ID NOs: 72, 93, 127, 161, WAS, and SEQ ID NO: 201; SEQ ID NOs: 69, 94, 124, 158, KIS, and SEQ ID NO: 198; SEQ ID NOs: 73, 95, 128, 162, AAS, and SEQ ID NO: 202; SEQ ID NOs: 74, 96, 129, 163, 185, and 203; SEQ ID NOs: 75, 97, 130, 164, 185, and 204; SEQ ID NOs: 74, 98, 131, 165, 185, and 205; SEQ ID NOs: 76, 99, 132, 166, 186, and 206; SEQ ID NOs: 77, 100, 133, 167, 187, and 207; SEQ ID NOs: 78, 101, 134, 168, 188, and 208; SEQ ID NOs: 79, 102, 135, 169, 186, and 209; SEQ ID NOs: 74, 103, 136, 170, 185, and 198; SEQ ID NOs: 80, 104, 137, 169, 189, and 210; SEQ ID NOs: 74, 105, 138, 170, 185, and 211; SEQ ID NOs: 74, 106, 139, 171, 187, and 212; SEQ ID NOs: 81, 107, 140, 172, 187, and 213; SEQ ID NOs: 81, 108, 141, 173, 190, and 214; SEQ ID NOs: 82, 109, 142, 174, 191, and 215; SEQ ID NOs: 83, 110, 143, 175, 186, and 216; SEQ ID NOs: 84, 99, 144, 176, 186, and 217; SEQ ID NOs: 85, 111, 145, 170, 192, and 198; SEQ ID NOs: 81, 112, 146, 171, 193, and 218; SEQ ID NOs: 86, 113, 147, 177, 187, and 219; SEQ ID NOs: 87, 114, 148, 178, 189, and 220; SEQ ID NOs: 74, 115, 149, 179, 187, and 221; SEQ ID NOs: 74, 116, 150, 165, 185, and 222; SEQ ID NOs: 74, 117, 151, 180, 187, and 223; SEQ ID NOs: 88, 118, 152, 181, 194, and 224; SEQ ID NOs: 89, 119, 153, 182, 195, and 199; SEQ ID NOs: 81, 120, 154, 172, 187, and 225; SEQ ID NOs: 74, 121, 155, 165, 185, and 222; SEQ ID NOs: 85, 122, 156, 183, 196, and 226; or SEQ ID NOs: 86, 123, 157, 184, 197, and 227, respectively.

In certain embodiments, the instant disclosure provides an antibody that specifically binds to LY6K (e.g., human LY6K) comprising a VH comprising an amino acid sequence that is at least 75%, 80%, 85%, 90%, 95%, or 100% (e.g., at least 86, 87, 88, 89, 90, 91, 92, 93, 94, 95, 96, 97, 98, or 99%) identical to the amino acid sequence set forth in any one of SEQ ID NOs: 1-34. In certain embodiments, the instant disclosure provides an antibody that specifically binds to LY6K (e.g., human LY6K), comprising a VH comprising an amino acid sequence set forth in any one of SEQ ID NOs: 1-34. In certain embodiments, the amino acid sequence of the VH consists of the amino acid sequence set forth in any one of SEQ ID NOs: 1-34.

In certain embodiments, the instant disclosure provides an antibody that specifically binds to LY6K (e.g., human LY6K), comprising a VL comprising an amino acid sequence that is at least 75%, 80%, 85%, 90%, 95%, or 100% (e.g., at least 86, 87, 88, 89, 90, 91, 92, 93, 94, 95, 96, 97, 98, or 99%) identical to the amino acid sequence set forth in any one of SEQ ID NOs: 35-68. In certain embodiments, the instant disclosure provides an antibody that specifically binds to LY6K (e.g., human LY6K), comprising a VL comprising an amino acid sequence set forth in any one of SEQ ID NOs: 35-68. In certain embodiments, the amino acid sequence of the VL consists of the amino acid sequence set forth in any one of SEQ ID NOs: 35-68.

In certain embodiments, the instant disclosure provides an antibody that specifically binds to LY6K (e.g., human LY6K), comprising a VH comprising an amino acid sequence that is at least 75%, 80%, 85%, 90%, 95%, or 100% (e.g., at least 86, 87, 88, 89, 90, 91, 92, 93, 94, 95, 96, 97, 98 or 99%) identical to the amino acid sequence set forth in any one of SEQ ID NOs: 1-34, and a VL comprising an amino acid sequence that is at least 75%, 80%, 85%, 90%, 95%, or 100% (e.g., at least 86, 87, 88, 89, 90, 91, 92, 93, 94, 95, 96, 97, 98, or 99%) identical to the amino acid sequence set forth in any one of SEQ ID NOs: 35-68. In certain embodiments, the instant disclosure provides an antibody that specifically binds to LY6K (e.g., human LY6K), comprising a VH comprising an amino acid sequence of any one of SEQ ID NOs: 1-34, and a VL comprising an amino acid sequence of any one of SEQ ID NOs: 35-68. In certain embodiments, the amino acid sequence of the VH consists of the amino acid sequence set forth in any one of SEQ ID NOs: 1-34, and the amino acid sequence of the VL consists of the amino acid sequence set forth in any one of SEQ ID NOs: 35-68.

In certain embodiments, the instant disclosure provides an antibody that specifically binds to LY6K (e.g., human LY6K), comprising a VH comprising an amino acid sequence that is at least 75%, 80%, 85%, 90%, 95%, or 100% (e.g., at least 86, 87, 88, 89, 90, 91, 92, 93, 94, 95, 96, 97, 98 or 99%) identical to an amino acid sequence set forth in any one of SEQ ID NOs: 1-34, and a VL comprising an amino acid sequence that is at least 75%, 80%, 85%, 90%, 95%, or 100% (e.g., at least 86, 87, 88, 89, 90, 91, 92, 93, 94, 95, 96, 97, 98, or 99%) identical to an amino acid sequence set forth in any one of SEQ ID NOs: 35-68, wherein the amino acid sequences set forth in any one of SEQ ID NOs: 1-34 and any one of SEQ ID NOs: 35-68 are from the same antibody (i.e., as shown in Tables 1 and 2). In certain embodiments, the instant disclosure provides an antibody that specifically binds to LY6K (e.g., human LY6K), comprising a VH comprising an amino acid sequence of any one of SEQ ID NOs: 1-34, and a VL comprising an amino acid sequence of any one of SEQ ID NOs: 35-68, wherein the amino acid sequences set forth in any one of SEQ ID NOs: 1-34 and any one of SEQ ID NOs: 35-68 are from the same antibody (i.e., as shown in Tables 1 and 2). In certain embodiments, the amino acid sequence of the VH consists of the amino acid sequence set forth in any one of SEQ ID NOs: 1-34, and the amino acid sequence of the VL consists of the amino acid sequence set forth in any one of SEQ ID NOs: 35-68, wherein the amino acid sequences set forth in any one of SEQ ID NOs: 1-34 and any one of SEQ ID NOs: 35-68 are from the same antibody (i.e., as shown in Tables 1 and 2).

In certain embodiments, the instant disclosure provides an antibody that specifically binds to LY6K (e.g., human LY6K), wherein the antibody comprises a VH and VL comprising the amino acid sequences set forth in SEQ ID NOs: 1 and 35; SEQ ID NOs: 2 and 36; SEQ ID NOs: 3 and 37; SEQ ID NOs: 4 and 38; SEQ ID NOs: 5 and 39; SEQ ID NOs: 6 and 40; SEQ ID NOs: 7 and 41; SEQ ID NOs: 8 and 42; SEQ ID NOs: 9 and 43; SEQ ID NOs: 10 and 44; SEQ ID NOs: 11 and 45; SEQ ID NOs: 12 and 46; SEQ ID NOs: 13 and 47; SEQ ID NOs: 14 and 48; SEQ ID NOs: 15 and 49; SEQ ID NOs: 16 and 50; SEQ ID NOs: 17 and 51; SEQ ID NOs: 18 and 52; SEQ ID NOs: 19 and 53; SEQ ID NOs: 20 and 54; SEQ ID NOs: 21 and 55; SEQ ID NOs: 22 and 56; SEQ ID NOs: 23 and 57; SEQ ID NOs: 24 and 58; SEQ ID NOs: 25 and 59; SEQ ID NOs: 26 and 60; SEQ ID NOs: 27 and 61; SEQ ID NOs: 28 and 62; SEQ ID NOs: 29 and 63; SEQ ID NOs: 30 and 64; SEQ ID NOs: 31 and 65; SEQ ID NOs: 32 and 66; SEQ ID NOs: 33 and 67; or SEQ ID NOs: 34 and 68, respectively.

In certain embodiments, the instant disclosure provides an antibody that specifically binds to LY6K (e.g., human LY6K), wherein the antibody comprises a VH and VL consisting of the amino acid sequences set forth in SEQ ID NOs: 1 and 35; SEQ ID NOs: 2 and 36; SEQ ID NOs: 3 and 37; SEQ ID NOs: 4 and 38; SEQ ID NOs: 5 and 39; SEQ ID NOs: 6 and 40; SEQ ID NOs: 7 and 41; SEQ ID NOs: 8 and 42; SEQ ID NOs: 9 and 43; SEQ ID NOs: 10 and 44; SEQ ID NOs: 11 and 45; SEQ ID NOs: 12 and 46; SEQ ID NOs: 13 and 47; SEQ ID NOs: 14 and 48; SEQ ID NOs: 15 and 49; SEQ ID NOs: 16 and 50; SEQ ID NOs: 17 and 51; SEQ ID NOs: 18 and 52; SEQ ID NOs: 19 and 53; SEQ ID NOs: 20 and 54; SEQ ID NOs: 21 and 55; SEQ ID NOs: 22 and 56; SEQ ID NOs: 23 and 57; SEQ ID NOs: 24 and 58; SEQ ID NOs: 25 and 59; SEQ ID NOs: 26 and 60; SEQ ID NOs: 27 and 61; SEQ ID NOs: 28 and 62; SEQ ID NOs: 29 and 63; SEQ ID NOs: 30 and 64; SEQ ID NOs: 31 and 65; SEQ ID NOs: 32 and 66; SEQ ID NOs: 33 and 67; or SEQ ID NOs: 34 and 68, respectively.

In certain embodiments, the instant disclosure provides an antibody that cross-competes for binding to LY6K (e.g., human LY6K) with any of the antibodies described above. In certain embodiments, the instant disclosure provides an antibody that binds to the same or an overlapping epitope of LY6K (e.g., an epitope of human LY6K) as an antibody described above.

In certain embodiments, the epitope of an antibody can be determined by, e.g., NMR spectroscopy, surface plasmon resonance (BIAcore®), X-ray diffraction crystallography studies, ELISA assays, hydrogen/deuterium exchange coupled with mass spectrometry (e.g., liquid chromatography electrospray mass spectrometry), array-based oligo-peptide scanning assays, and/or mutagenesis mapping (e.g., site-directed mutagenesis mapping). For X-ray crystallography, crystallization may be accomplished using any of the known methods in the art (e.g., Giege R et al., (1994) Acta Crystallogr D Biol Crystallogr 50 (Pt 4): 339-350; McPherson A (1990) Eur J Biochem 189: 1-23; Chayen N E (1997) Structure 5: 1269-1274; McPherson A (1976) J Biol Chem 251: 6300-6303, all of which are herein incorporated by reference in their entireties). Antibody:antigen crystals may be studied using well known X-ray diffraction techniques and may be refined using computer software such as X-PLOR (Yale University, 1992, distributed by Molecular Simulations, Inc.; see, e.g., Meth Enzymol (1985) volumes 114 & 115, eds. Wyckoff H W et al.; U.S. Patent Application No. 2004/0014194), and BUSTER (Bricogne G (1993) Acta Crystallogr D Biol Crystallogr 49 (Pt 1): 37-60; Bricogne G (1997) Meth Enzymol 276A: 361-423, ed Carter C W; Roversi P et al., (2000) Acta Crystallogr D Biol Crystallogr 56 (Pt 10): 1316-1323, all of which are herein incorporated by reference in their entireties). Mutagenesis mapping studies may be accomplished using any method known to one of skill in the art. See, e.g., Champe M et al., (1995) supra and Cunningham B C & Wells J A (1989) supra for a description of mutagenesis techniques, including alanine scanning mutagenesis techniques. In a specific embodiment, the epitope of an antibody is determined using alanine scanning mutagenesis studies. In addition, or antibodies that recognize and bind to the same or overlapping epitopes of LY6K (e.g., human LY6K) can be identified using routine techniques such as an immunoassay, for example, by showing the ability of one antibody to block the binding of another antibody to a target antigen, i.e., a competitive binding assay. Competition binding assays also can be used to determine whether two antibodies have similar binding specificity for an epitope. Competitive binding can be determined in an assay in which the immunoglobulin under test inhibits specific binding of a reference antibody to a common antigen, such as LY6K (e.g., human LY6K). Numerous types of competitive binding assays are known, for example: solid phase direct or indirect radioimmunoassay (RIA), solid phase direct or indirect enzyme immunoassay (EIA), sandwich competition assay (see Stahli C et al., (1983) Methods Enzymol 9: 242-253); solid phase direct biotin-avidin EIA (see Kirkland T N et al., (1986) J Immunol 137: 3614-9); solid phase direct labeled assay, solid phase direct labeled sandwich assay (see Harlow E & Lane D, (1988) Antibodies: A Laboratory Manual, Cold Spring Harbor Press); solid phase direct label RIA using I-125 label (see Morel G A et al., (1988) Mol Immunol 25(1): 7-15); solid phase direct biotin-avidin EIA (see Cheung R C et al., (1990) Virology 176: 546-52); and direct labeled RIA (see Moldenhauer G et al., (1990) Scand J Immunol 32: 77-82), all of which are herein incorporated by reference in their entireties. Typically, such an assay involves the use of purified antigen (e.g., LY6K, such as human LY6K) bound to a solid surface or cells bearing either of these, an unlabeled test immunoglobulin and a labeled reference immunoglobulin. Competitive inhibition can be measured by determining the amount of label bound to the solid surface or cells in the presence of the test immunoglobulin. Usually, the test immunoglobulin is present in excess. Usually, when a competing antibody is present in excess, it will inhibit specific binding of a reference or antibody to a common antigen by at least 50-55%, 55-60%, 60-65%, 65-70%, 70-75%, or more. A competition binding assay can be configured in a large number of different formats using either labeled antigen or labeled antibody. In a common version of this assay, the antigen is immobilized on a 96-well plate. The ability of unlabeled antibodies to block the binding of labeled antibodies to the antigen is then measured using radioactive or enzyme labels. For further details see, e.g., Wagener C et al., (1983) J Immunol 130: 2308-2315; Wagener C et al., (1984) J Immunol Methods 68: 269-274; Kuroki M et al., (1990) Cancer Res 50: 4872-4879; Kuroki M et al., (1992) Immunol Invest 21: 523-538; Kuroki M et al., (1992) Hybridoma 11: 391-407 and Antibodies: A Laboratory Manual, Ed Harlow E & Lane D editors supra, pp. 386-389, all of which are herein incorporated by reference in their entireties.

The anti-LY6K antigen-binding molecules of the present disclosure can be linked to or co-expressed with another functional molecule, e.g., another peptide or protein. For example, an antibody or fragment thereof can be functionally linked (e.g., by chemical coupling, genetic fusion, noncovalent association, or otherwise) to one or more other molecular entities, such as another antibody or antibody fragment to produce a bispecific or a multispecific antibody (e.g., a bispecific T cell engager [BiTE] or a dual-affinity re-targeting antibody [DART]) with a second or additional binding specificity. In some embodiments, the bispecific or multispecific antibody has binding specificity for a molecule on an effector cell (e.g., CD3, CD16, CD137). In some embodiments, an antibody provided herein is a multispecific antibody. In some embodiments, a multispecific antibody provided herein comprises a CD3-binding region. In some embodiments, the CD3-binding region comprises a sequence from an antibody known to bind CD3 (e.g., OKT3, UTCH1, L2K, TR66, etc.). In some embodiments, the CD3-binding region comprises a sequence from an L2K anti-CD3 antibody. In some embodiments, the CD3-binding region comprises a sequence from a de-immunized L2K anti-CD3 antibody. In some embodiments, the CD3-binding region comprises a single-chain fragment variable (scFv) that specifically binds to CD3. In some embodiments, a multispecific antibody provided herein specifically binds to human CD3.

In certain embodiments, an antibody disclosed herein is conjugated to a pharmaceutically active substance. Pharmaceutically active substances include, but are not limited to, cytotoxic agents, cytostatic agents, toxins, radionuclides (e.g., radioisotopes), polypeptides, polynucleotides, detectable labels, and combinations thereof. In certain embodiments, pharmaceutically active substance is a cytotoxic agent. In certain embodiments, the cytotoxic agent is able to induce death or destruction of a cell in contact therewith. In certain embodiments, the pharmaceutically active substance is a cytostatic agent. In certain embodiments, the cytostatic agent is able to prevent or substantially reduce proliferation and/or inhibits the activity or function of a cell in contact therewith. In certain embodiments, the cytotoxic agent or cytostatic agent is a chemotherapeutic agent.

In certain embodiments, the pharmaceutically active substance is a radionuclide. Suitable radionuclides include, but are not limited to, beta emitters, auger emitters, converted electron emitters, alpha emitters, and low photon energy emitters. In certain embodiments, the radionuclide is selected from ³H, ¹⁴C, ³²P, ³⁵S, ³⁶Cl, ⁴⁵Ca, ⁵¹Cr, ⁵⁷Co, ⁵⁸Co, ⁵⁹Fe, ⁶⁷Cu, ⁶⁷Ga, ⁷⁶As, ⁷⁷As, ⁸⁹Sr, ⁹⁰Y, ⁹⁹Tc, ^{99m}Tc, ¹⁰⁵Rh, ¹¹¹In, ^{114m}In, ¹¹⁷Lu, ¹²¹I, ¹²³I, ¹²⁴I, ¹²⁵I, ¹³¹I, ¹⁴⁹Tb, ¹⁵³Sm, ¹⁶¹Tb, ¹⁶⁶Ho, ¹⁷⁷Lu, ¹⁹⁸Au, ²⁰¹Tl, ²¹¹At, ²¹²Pb, ²¹³Bi, ²²⁵Ac, ¹⁸⁶Re, ¹⁸⁸Re, ²¹²Bi, ²¹³Bi, ²²¹At, ²²³Ac, ²²³Ra, ²²⁵Ac, ²⁵⁵Fm, and combinations thereof.

In certain embodiments, the pharmaceutically active substance is a detectable label. In certain embodiments, the detectable label comprises a fluorescent moiety, a click chemistry handle, or a combination thereof.

In certain embodiments, the pharmaceutically active substance is a drug. Suitable drugs include, but are not limited to, anti-cancer agents, anti-inflammatory agents, and anti-infective (e.g., anti-fungal, antibacterial, anti-parasitic, antiviral) agents. Suitable anti-cancer agents include, but are not limited to, alkylating agents, antimetabolites, spindle poison plant alkaloids, cytotoxic/antitumor antibiotics, topoisomerase inhibitors, photosensitizers, kinase inhibitors, anti-hormonal agents, aromatase inhibitors, anti-androgens, protein kinase inhibitors, lipid kinase inhibitors, antisense oligonucleotides (e.g., those which inhibit expression of genes in signaling pathways implicated in aberrant cell proliferation), ribozymes, (e.g., VEGF expression inhibitors and HER2 expression inhibitors), vaccines (e.g., gene therapy vaccines), topoisomerase 1 inhibitors, anti-angiogenic agents, pharmaceutically acceptable salts, acids, solvates and derivatives of any of the above, and any combination thereof.

In certain embodiments, the pharmaceutically active substance is a toxin. Suitable toxins include, but are not limited to, proteinaceous toxins (e.g., bacterial-derived toxins, and plant-derived toxins), toxins targeting tubulin filaments, toxins targeting DNA, toxins targeting RNA. Examples of proteinaceous toxins include saporin, dianthin, ricin, modeccin, abrin, volkensin, viscumin, shiga toxin, shiga-like toxin, pseudomonas exotoxin (PE, also known as exotoxin A), diphtheria toxin (DT), and cholera toxin. Examples of toxins targeting tubulin filaments include maytansinoids (e.g. DM1 and DM4), auristatins (e.g., Monomethyl auristatin E (MMAE) and Monomethyl auristatin F (MMAF)), toxoids, tubulysins, cryptophycins, rhizoxin. Examples of DNA-targeting toxins include calicheamicins: N-Acetyl-y-calicheamicin, CC-1065 analogs, duocarmycins, doxorubicin, methotrexate, benzodiazepines, camptothecin analogues, and anthracyclines. Examples of RNA-targeting toxins are amanitins, spliceostatins, and thailanstatins.

In certain embodiments, the pharmaceutically active substance is a polypeptide. Suitable polypeptides include, but are not limited to, Cas9; toxins (e.g., saporin, dianthin, gelonin, (de)bouganin, agrostin, ricin (toxin A chain); pokeweed antiviral protein, apoptin, diphtheria toxin, pseudomonas exotoxin); metabolic enzymes (e.g., argininosuccinate lyase, argininosuccinate synthetase); enzymes of the coagulation cascade; repairing enzymes; enzymes for cell signaling; cell cycle regulation factors; gene regulating factors (e.g., ranscription factors such as NF-KB or gene repressors such as methionine repressor).

In certain embodiments, the pharmaceutically active substance is a polynucleotide. In certain embodiments, the polynucleotide comprises coding information. In certain embodiments, the polynucleotide is a gene or an open reading frame encoding a protein. In certain embodiments, the polynucleotide comprises regulatory information. In certain embodiments, the polynucleotide is a promoter, a regulatory element binding region, or a sequence encoding a micro RNA. Suitable polynucleotides include natural and artificial nucleic acids. Artificial nucleic acids include, but are not limited to, peptide nucleic acids (PNA), Morpholinos and locked nucleic acids (LNA), glycol nucleic acids (GNA), and threose nucleic acids (TNA). Each of these is distinguished from naturally occurring DNA or RNA by changes to the backbone of the molecule. Suitable polynucleotides include, but are not limited to, a vector; a gene (e.g., a cell suicide-inducing transgene); single stranded DNA; linear double stranded DNA; circular double stranded DNA (e.g., a plasmid); mini-circle DNA; a DNA aptamer; single stranded RNA; linear double stranded RNA; mRNA; tRNA; rRNA; short interfering RNA (siRNA); microRNA (miRNA); antisense RNA; anti-sense oligonucleotides; peptide nucleic acid (PNA); phosphoramidate morpholino oligomer (PMO); locked nucleic acid (LNA); bridged nucleic acid (BNA); 2′-deoxy-2′-fluoroarabino nucleic acid (FANA); 2′-O-methoxyethyl-RNA (MOE); 2′-0,4′-aminoethylene bridged nucleic acid; 3′-fluoro hexitol nucleic acid (FHNA); an RNA aptamer; and combinations thereof.

Any immunoglobulin (Ig) constant region can be used in the antibodies disclosed herein. In certain embodiments, the Ig region is a human IgG, IgE, IgM, IgD, IgA, or IgY immunoglobulin molecule, any class (e.g., IgG₁, IgG₂, IgG₃, IgG₄, IgA₁, and IgA₂), or any subclass (e.g., IgG₂a and IgG₂b) of immunoglobulin molecule.

In certain embodiments, the instant disclosure provides an antibody that specifically binds to LY6K (e.g., human LY6K), the antibody comprising a heavy chain constant region, optionally selected from the group consisting of human IgG₁, IgG₂, IgG₃, IgG₄, IgA₁, and IgA₂.

In certain embodiments, the instant disclosure provides an antibody that specifically binds to LY6K (e.g., human LY6K), the antibody comprising a heavy chain constant region that is a variant of a wild-type heavy chain constant region, wherein the variant heavy chain constant region binds to an FcγR with lower affinity than the wild-type heavy chain constant region binds to the FcγR.

In certain embodiments, the instant disclosure provides an antibody that specifically binds to LY6K (e.g., human LY6K), the antibody comprising a heavy chain constant region comprising an amino acid sequence shown in Table 5. In certain embodiments, the heavy chain constant region comprises the amino acid sequence of SEQ ID NO: 228 or 229. In certain embodiments, the heavy chain constant region consists of the amino acid sequence of SEQ ID NO: 228 or 229.

TABLE 5
Heavy chain constant region amino acid
sequences of exemplary anti-LY6K antibodies.

| | | SEQ |
|---|---|---|
| Description | Amino Acid Sequence | ID |
| WT IgG1 heavy | ASTKGPSVFPLAPSSKSTSGGTAAL | 228 |
| chain constant | GCLVKDYFPEPVTVSWNSGALTSGV | |
| region | HTFPAVLQSSGLYSLSSVVTVPSSS | |
| | LGTQTYICNVNHKPSNTKVDKKVEP | |
| | KSCDKTHTCPPCPAPELLGGPSVFL | |
| | FPPKPKDTLMISRTPEVTCVVVDVS | |
| | HEDPEVKFNWYVDGVEVHNAKTKPR | |
| | EEQYNSTYRVVSVLTVLHQDWLNGK | |
| | EYKCKVSNKALPAPIEKTISKAKGQ | |
| | PREPQVYTLPPSRDELTKNQVSLTC | |
| | LVKGFYPSDIAVEWESNGQPENNYK | |
| | TTPPVLDSDGSFFLYSKLTVDKSRW | |
| | QQGNVFSCSVMHEALHNHYTQKSLS | |
| | LSPGK | |
| S228P IgG4 | ASTKGPSVFPLAPCSRSTSESTAAL | 229 |
| heavy chain | GCLVKDYFPEPVTVSWNSGALTSGV | |
| constant | HTFPAVLQSSGLYSLSSVVTVPSSS | |
| region | LGTKTYTCNVDHKPSNTKVDKRVES | |
| | KYGPPCPPCPAPEFLGGPSVFLFPP | |
| | KPKDTLMISRTPEVTCVVVDVSQED | |
| | PEVQFNWYVDGVEVHNAKTKPREEQ | |
| | FNSTYRVVSVLTVLHQDWLNGKEYK | |
| | CKVSNKGLPSSIEKTISKAKGQPRE | |
| | PQVYTLPPSQEEMTKNQVSLTCLVK | |
| | GFYPSDIAVEWESNGQPENNYKTTP | |
| | PVLDSDGSFFLYSRLTVDKSRWQEG | |
| | NVFSCSVMHEALHNHYTQKSLSLSL | |
| | GK | |

In certain embodiments, the instant disclosure provides an antibody that specifically binds to LY6K (e.g., human LY6K), the antibody comprising a light chain constant region comprising an amino acid sequence shown in Table 6. In certain embodiments, the heavy chain constant region comprises the amino acid sequence of SEQ ID NO: 230 or 231. In certain embodiments, the heavy chain constant region consists of the amino acid sequence of SEQ ID NO: 230 or 231.

TABLE 6
Light chain constant region amino acid
sequences of exemplary anti-LY6K antibodies.

| | | SEQ |
|---|---|---|
| Description | Amino Acid Sequence | ID |
| Kappa light | RTVAAPSVFIFPPSDEQLKSGTASV | 230 |
| chain constant | VCLLNNFYPREAKVQWKVDNALQSG | |
| region | NSQESVTEQDSKDSTYSLSSTLTLS | |
| | KADYEKHKVYACEVTHQGLSSPVTK | |
| | SFNRGEC | |
| Lambda light | GQPKANPTVTLFPPSSEELQANKAT | 231 |
| chain constant | LVCLISDFYPGAVTVAWKADGSPVK | |
| region | AGVETTKPSKQSNNKYAASSYLSLT | |
| | PEQWKSHRSYSCQVTHEGSTVEKTV | |
| | APTECS | |

In certain embodiments, one, two, or more mutations (e.g., amino acid substitutions) are introduced into an Fc region (e.g., a CH2 domain (residues 231-340 of human IgG₁)) and/or a CH3 domain (residues 341-447 of human IgG₁, numbered according to the EU numbering system) and/or a hinge region (residues 216-230, numbered according to the EU numbering system) of an antibody described herein, to alter one or more functional properties of the antibody, such as serum half-life, complement fixation, Fc receptor binding, and/or antigen-dependent cellular cytotoxicity.

In certain embodiments, one, two, or more mutations (e.g., amino acid substitutions) are introduced into the hinge region of an antibody described herein, such that the number of cysteine residues in the hinge region is altered (e.g., increased or decreased) as described in, e.g., U.S. Pat. No. 5,677,425, herein incorporated by reference in its entirety. The number of cysteine residues in the hinge region may be altered to, e.g., facilitate assembly of the light and heavy chains, or to alter (e.g., increase or decrease) the stability of the antibody.

In a specific embodiment, one, two, or more amino acid mutations (e.g., substitutions, insertions, or deletions) are introduced into an IgG constant region, or FcRn-binding fragment thereof (preferably an Fc or hinge-Fc fragment) to alter (e.g., decrease or increase) half-life of the antibody in vivo. See, e.g., International Publication Nos. WO 02/060919; WO 98/23289; and WO 97/34631; and U.S. Pat. Nos. 5,869,046, 6,121,022, 6,277,375, and 6,165,745, all of which are herein incorporated by reference in their entireties, for examples of mutations that will alter (e.g., decrease or increase) the half-life of an antibody in vivo. In certain embodiments, one, two or more amino acid mutations (e.g., substitutions, insertions, or deletions) are introduced into an IgG constant region, or FcRn-binding fragment thereof (preferably an Fc or hinge-Fc fragment) to decrease the half-life of the antibody in vivo. In other embodiments, one, two or more amino acid mutations (e.g., substitutions, insertions, or deletions) are introduced into an IgG constant region, or FcRn-binding fragment thereof (preferably an Fc or hinge-Fc fragment) to increase the half-life of the antibody in vivo. In a specific embodiment, the antibodies may have one or more amino acid mutations (e.g., substitutions) in the second constant (CH2) domain (residues 231-340 of human IgG₁) and/or the third constant (CH3) domain (residues 341-447 of human IgG₁), numbered according to the EU numbering system. In a specific embodiment, the constant region of the IgG₁ of antibody described herein comprises a methionine (M) to tyrosine (Y) substitution in position 252, a serine (S) to threonine (T) substitution in position 254, and a threonine (T) to glutamic acid (E) substitution in position 256, numbered according to the EU numbering system. See U.S. Pat. No. 7,658,921, which is herein incorporated by reference in its entirety. This type of mutant IgG, referred to as “YTE mutant” has been shown to display fourfold increased half-life as compared to wild-type versions of the same antibody (see Dall'Acqua W F et al., (2006) J Biol Chem 281: 23514-24, which is herein incorporated by reference in its entirety). In certain embodiments, an antibody comprises an IgG constant region comprising one, two, three, or more amino acid substitutions of amino acid residues at positions 251-257, 285-290, 308-314, 385-389, and 428-436, numbered according to the EU numbering system.

In certain embodiments, one, two, or more mutations (e.g., amino acid substitutions) are introduced into an Fc region (e.g., a CH2 domain (residues 231-340 of human IgG₁)) and/or a CH3 domain (residues 341-447 of human IgG₁, numbered according to the EU numbering system) and/or a hinge region (residues 216-230, numbered according to the EU numbering system) of an antibody described herein, to increase or decrease the affinity of the antibody for an Fc receptor (e.g., an activated Fc receptor) on the surface of an effector cell. Mutations in the Fc region of an antibody that decrease or increase the affinity of an antibody for an Fc receptor and techniques for introducing such mutations into the Fc receptor or fragment thereof are known to one of skill in the art. Examples of mutations in the Fc receptor of an antibody that can be made to alter the affinity of the antibody for an Fc receptor are described in, e.g., Smith P et al., (2012) PNAS 109: 6181-6186, U.S. Pat. No. 6,737,056, and International Publication Nos. WO 02/060919; WO 98/23289; and WO 97/34631, all of which are herein incorporated by reference in their entireties.

In certain embodiments, the antibody comprises a heavy chain constant region that is a variant of a wild-type heavy chain constant region, wherein the variant heavy chain constant region binds to FcγRIIB with higher affinity than the wild-type heavy chain constant region binds to FcγRIIB. In certain embodiments, the variant heavy chain constant region is a variant human heavy chain constant region, e.g., a variant human IgG₁, a variant human IgG₂, or a variant human IgG₄ heavy chain constant region. In certain embodiments, the variant human IgG heavy chain constant region comprises one or more of the following amino acid mutations, according to the EU numbering system: G236D, P238D, S239D, S267E, L328F, and L328E. In certain embodiments, the variant human IgG heavy chain constant region comprises a set of amino acid mutations selected from the group consisting of S267E and L328F; P238D and L328E; P238D and one or more substitutions selected from the group consisting of E233D, G237D, H268D, P271G, and A330R; P238D, E233D, G237D, H268D, P271G, and A330R; G236D and S267E; S239D and S267E; V262E, S267E, and L328F; and V264E, S267E, and L328F, according to the EU numbering system. In certain embodiments, the FcγRIIB is expressed on a cell selected from the group consisting of macrophages, monocytes, B cells, dendritic cells, endothelial cells, and activated T cells.

In a further embodiment, one, two, or more amino acid substitutions are introduced into an IgG constant region Fc region to alter the effector function(s) of the antibody. For example, one or more amino acids selected from amino acid residues 234, 235, 236, 237, 239, 243, 267, 292, 297, 300, 318, 320, 322, 328, 330, 332, and 396, numbered according to the EU numbering system, can be replaced with a different amino acid residue such that the antibody has an altered affinity for an effector ligand but retains the antigen-binding ability of the parent antibody. The effector ligand to which affinity is altered can be, for example, an Fc receptor or the C1 component of complement. This approach is described in further detail in U.S. Pat. Nos. 5,624,821 and 5,648,260, each of which is herein incorporated by reference in its entirety. In certain embodiments, the deletion or inactivation (through point mutations or other means) of a constant region domain may reduce Fc receptor binding of the circulating antibody thereby increasing tumor localization. See, e.g., U.S. Pat. Nos. 5,585,097 and 8,591,886, each of which is herein incorporated by reference in its entirety, for a description of mutations that delete or inactivate the constant region and thereby increase tumor localization. In certain embodiments, one or more amino acid substitutions may be introduced into the Fc region of an antibody described herein to remove potential glycosylation sites on the Fc region, which may reduce Fc receptor binding (see, e.g., Shields R L et al., (2001) J Biol Chem 276: 6591-604, which is herein incorporated by reference in its entirety). In various embodiments, one or more of the following mutations in the constant region of an antibody described herein may be made: an N297A substitution; an N297Q substitution; an L234A substitution; an L234F substitution; an L235A substitution; an L235F substitution; an L235V substitution; an L237A substitution; an S239D substitution; an E233P substitution; an L234V substitution; a C236 deletion; a P238A substitution; an F243L substitution; a D265A substitution; an S267E substitution; an L328F substitution; an R292P substitution; a Y300L substitution; an A327Q substitution; a P329A substitution; an A330L substitution; an 1332E substitution; or a P396L substitution, numbered according to the EU numbering system.

In certain embodiments, a mutation selected from the group consisting of D265A, P329A, and a combination thereof, numbered according to the EU numbering system, may be made in the constant region of an antibody described herein. In certain embodiments, a mutation selected from the group consisting of L235A, L237A, and a combination thereof, numbered according to the EU numbering system, may be made in the constant region of an antibody described herein. In certain embodiments, a mutation selected from the group consisting of S267E, L328F, and a combination thereof, numbered according to the EU numbering system, may be made in the constant region of an antibody described herein. In certain embodiments, a mutation selected from the group consisting of S239D, 1332E, optionally A330L, and a combination thereof, numbered according to the EU numbering system, may be made in the constant region of an antibody described herein. In certain embodiments, a mutation selected from the group consisting of L235V, F243L, R292P, Y300L, P396L, and a combination thereof, numbered according to the EU numbering system, may be made in the constant region of an antibody described herein. In certain embodiments, a mutation selected from the group consisting of S267E, L328F, and a combination thereof, numbered according to the EU numbering system, may be made in the constant region of an antibody described herein.

In a specific embodiment, an antibody described herein comprises the constant region of an IgG₁ with an N297Q or N297A amino acid substitution, numbered according to the EU numbering system. In certain embodiments, an antibody described herein comprises the constant region of an IgG₁ with a mutation selected from the group consisting of D265A, P329A, and a combination thereof, numbered according to the EU numbering system. In another embodiment, an antibody described herein comprises the constant region of an IgG₁ with a mutation selected from the group consisting of L234A, L235A, and a combination thereof, numbered according to the EU numbering system. In another embodiment, an antibody described herein comprises the constant region of an IgG₁ with a mutation selected from the group consisting of L234F, L235F, N297A, and a combination thereof, numbered according to the EU numbering system. In certain embodiments, amino acid residues in the constant region of an antibody described herein in the positions corresponding to positions L234, L235, and D265 in a human IgG₁ heavy chain, numbered according to the EU numbering system, are not L, L, and D, respectively. This approach is described in detail in International Publication No. WO 14/108483, which is herein incorporated by reference in its entirety. In certain embodiments, the amino acids corresponding to positions L234, L235, and D265 in a human IgG₁ heavy chain are F, E, and A; or A, A, and A, respectively, numbered according to the EU numbering system.

In certain embodiments, one or more amino acids selected from amino acid residues 329, 331, and 322 in the constant region of an antibody described herein, numbered according to the EU numbering system, can be replaced with a different amino acid residue such that the antibody has altered C1q binding and/or reduced or abolished complement dependent cytotoxicity (CDC). This approach is described in further detail in U.S. Pat. No. 6,194,551 (Idusogie et al.), which is herein incorporated by reference in its entirety. In certain embodiments, one or more amino acid residues within amino acid positions 231 to 238 in the N-terminal region of the CH2 domain of an antibody described herein are altered to thereby alter the ability of the antibody to fix complement, numbered according to the EU numbering system. This approach is described further in International Publication No. WO 94/29351, which is herein incorporated by reference in its entirety. In certain embodiments, the Fc region of an antibody described herein is modified to increase the ability of the antibody to mediate antibody dependent cellular cytotoxicity (ADCC) and/or to increase the affinity of the antibody for an Fcγ receptor by mutating one or more amino acids (e.g., introducing amino acid substitutions) at the following positions: 238, 239, 248, 249, 252, 254, 255, 256, 258, 265, 267, 268, 269, 270, 272, 276, 278, 280, 283, 285, 286, 289, 290, 292, 293, 294, 295, 296, 298, 301, 303, 305, 307, 309, 312, 315, 320, 322, 324, 326, 327, 328, 329, 330, 331, 333, 334, 335, 337, 338, 340, 360, 373, 376, 378, 382, 388, 389, 398, 414, 416, 419, 430, 434, 435, 437, 438, or 439, numbered according to the EU numbering system. This approach is described further in International Publication No. WO 00/42072, which is herein incorporated by reference in its entirety.

In certain embodiments, an antibody described herein comprises a modified constant region of an IgG₁, wherein the modification increases the ability of the antibody to mediate antibody dependent cellular cytotoxicity (ADCC). In certain embodiments, 0.1, 1, or 10 μg/ml of the antibody is capable of inducing cell death of at least 20%, 25%, 30%, 35%, 40%, 45%, 50%, 55%, or 60% of LY6K-expressing cells within 1, 2, or 3 hours, as assessed by methods described herein and/or known to a person of skill in the art. In certain embodiments, the modified constant region of an IgG₁ comprises S239D and 1332E substitutions, numbered according to the EU numbering system. In certain embodiments, the modified constant region of an IgG₁ comprises S239D, A330L, and 1332E substitutions, numbered according to the EU numbering system. In certain embodiments, the modified constant region of an IgG₁ comprises L235V, F243L, R292P, Y300L, and P396L substitutions, numbered according to the EU numbering system. In certain embodiments, the antibody is capable of inducing cell death in effector T cells and Tregs, wherein the percentage of Tregs that undergo cell death is higher than the percentage of effector T cells that undergo cell death by at least 1.2 fold, 1.3 fold, 1.4 fold, 1.5 fold, 1.6 fold, 1.7 fold, 1.8 fold, 1.9 fold, 2 fold, 2.5 fold, 3 fold, 3.5 fold, 4 fold, 4.5 fold, or 5 fold.

In certain embodiments, an antibody described herein comprises the constant region of an IgG₄ antibody and the serine at amino acid residue 228 of the heavy chain, numbered according to the EU numbering system, is substituted for proline.

In certain embodiments, any of the constant region mutations or modifications described herein can be introduced into one or both heavy chain constant regions of an antibody described herein having two heavy chain constant regions.

### Polypeptides

In another aspect, provided herein are polypeptides comprising one or more sequences set forth in Tables 1-4, above. In certain embodiments, the polypeptide comprises the CDRH1, CDRH2, and/or CDRH3 of a VH amino acid sequence set forth in any one of SEQ ID NOs: 1-34, as determined by any of the methods discussed above. In certain embodiments, the polypeptide comprises a VH comprising the CDRH1, CDRH2, and CDRH3 amino acid sequences of the VH amino acid sequence set forth in any one of SEQ ID NOs: 1-34. In certain embodiments, the polypeptide comprises the CDRH1, CDRH2, and CDRH3 amino acid sequences set forth in SEQ ID NOs: 69, 90, and 124; 70, 91, and 125; 71, 92, and 126; 72, 93, and 127; 69, 94, and 124; 73, 95, and 128; 74, 96, and 129; 75, 97, and 130; 74, 98, and 131; 76, 99, and 132; 77, 100, and 133; 78, 101, and 134; 79, 102, and 135; 74, 103, and 136; 80, 104, and 137; 74, 105, and 138; 74, 106, and 139; 81, 107, and 140; 81, 108, and 141; 82, 109, and 142; 83, 110, and 143; 84, 99, and 144; 85, 111, and 145; 81, 112, and 146; 86, 113, and 147; 87, 114, and 148; 74, 115, and 149; 74, 116, and 150; 74, 117, and 151; 88, 118, and 152; 89, 119, and 153; 81, 120, and 154; 74, 121, and 155; 85, 122, and 156; or 86, 123, and 157, respectively.

In certain embodiments, the polypeptide comprises the CDRL1, CDRL2, and/or CDRL3 of a VL amino acid sequence set forth in any one of SEQ ID NOs: 35-68, as determined by any of the methods discussed above. In certain embodiments, the polypeptide comprises a VL comprising the CDRL1, CDRL2, and CDRL3 amino acid sequences of the VL amino acid sequence set forth in any one of SEQ ID NOs: 35-68. In certain embodiments, the polypeptide comprises the CDRL1, CDRL2, and CDRL3 amino acid sequences set forth in SEQ ID NO: 158, KIS, and SEQ ID NO: 198; SEQ ID NO: 159, WAS, and SEQ ID NO: 199; SEQ ID NO: 160, STT, and SEQ ID NO: 200; SEQ ID NO: 161, WAS, and SEQ ID NO: 201; SEQ ID NO: 162, AAS, and SEQ ID NO: 202; SEQ ID NOs: 163, 185, and 203; SEQ ID NOs: 164, 185, and 204; SEQ ID NOs: 165, 185, and 205; SEQ ID NOs: 166, 186, and 206; SEQ ID NOs: 167, 187, and 207; SEQ ID NOs: 168, 188, and 208; SEQ ID NOs: 169, 186, and 209; SEQ ID NOs: 170, 185, and 198; SEQ ID NOs: 169, 189, and 210; SEQ ID NOs: 170, 185, and 211; SEQ ID NOs: 171, 187, and 212; SEQ ID NOs: 172, 187, and 213; SEQ ID NOs: 173, 190, and 214; SEQ ID NOs: 174, 191, and 215; SEQ ID NOs: 175, 186, and 216; SEQ ID NOs: 176, 186, and 217; SEQ ID NOs: 170, 192, and 198; SEQ ID NOs: 171, 193, and 218; SEQ ID NOs: 177, 187, and 219; SEQ ID NOs: 178, 189, and 220; SEQ ID NOs: 179, 187, and 221; SEQ ID NOs: 165, 185, and 222; SEQ ID NOs: 180, 187, and 223; SEQ ID NOs: 181, 194, and 224; SEQ ID NOs: 182, 195, and 199; SEQ ID NOs: 172, 187, and 225; SEQ ID NOs: 183, 196, and 226; or SEQ ID NOs: 184, 197, and 227, respectively.

In certain embodiments, the polypeptide comprises a VH comprising the CDRH1, CDRH2, and CDRH3 amino acid sequences of the VH amino acid sequence set forth in any one of SEQ ID NOs: 1-34; and a VL comprising the CDRL1, CDRL2, and CDRL3 amino acid sequences of the VL amino acid sequence set forth in any one of SEQ ID NOs: 35-68. In certain embodiments, the polypeptide comprises the CDRH1, CDRH2, CDRH3, CDRL1, CDRL2, and CDRL3 amino acid sequences of the VH and VL amino acid sequences set forth in SEQ ID NOs: 1 and 35, 2 and 36, 3 and 37, 4 and 38, 5 and 39, 6 and 40, 7 and 41, 8 and 42, 9 and 43, 10 and 44, 11 and 45, 12 and 46, 13 and 47, 14 and 48, 15 and 49, 16 and 50, 17 and 51, 18 and 52, 19 and 53, 20 and 54, 21 and 55, 22 and 56, 23 and 57, 24 and 58, 25 and 59, 26 and 60, 27 and 61, 28 and 62, 29 and 63, 30 and 64, 31 and 65, 32 and 66, 33 and 67, or 34 and 68, respectively.

In certain embodiments, a polypeptide provided herein comprises the CDRH1, CDRH2, CDRH3, CDRL1, CDRL2, and CDRL3 amino acid sequences set forth in: SEQ ID NOs: 69, 90, 124, 158, KIS, and SEQ ID NO: 198; SEQ ID NOs: 70, 91, 125, 159, WAS, and SEQ ID NO: 199; SEQ ID NOs: 71, 92, 126, 160, STT, and SEQ ID NO: 200; SEQ ID NOs: 72, 93, 127, 161, WAS, and SEQ ID NO: 201; SEQ ID NOs: 69, 94, 124, 158, KIS, and SEQ ID NO: 198; SEQ ID NOs: 73, 95, 128, 162, AAS, and SEQ ID NO: 202; SEQ ID NOs: 74, 96, 129, 163, 185, and 203; SEQ ID NOs: 75, 97, 130, 164, 185, and 204; SEQ ID NOs: 74, 98, 131, 165, 185, and 205; SEQ ID NOs: 76, 99, 132, 166, 186, and 206; SEQ ID NOs: 77, 100, 133, 167, 187, and 207; SEQ ID NOs: 78, 101, 134, 168, 188, and 208; SEQ ID NOs: 79, 102, 135, 169, 186, and 209; SEQ ID NOs: 74, 103, 136, 170, 185, and 198; SEQ ID NOs: 80, 104, 137, 169, 189, and 210; SEQ ID NOs: 74, 105, 138, 170, 185, and 211; SEQ ID NOs: 74, 106, 139, 171, 187, and 212; SEQ ID NOs: 81, 107, 140, 172, 187, and 213; SEQ ID NOs: 81, 108, 141, 173, 190, and 214; SEQ ID NOs: 82, 109, 142, 174, 191, and 215; SEQ ID NOs: 83, 110, 143, 175, 186, and 216; SEQ ID NOs: 84, 99, 144, 176, 186, and 217; SEQ ID NOs: 85, 111, 145, 170, 192, and 198; SEQ ID NOs: 81, 112, 146, 171, 193, and 218; SEQ ID NOs: 86, 113, 147, 177, 187, and 219; SEQ ID NOs: 87, 114, 148, 178, 189, and 220; SEQ ID NOs: 74, 115, 149, 179, 187, and 221; SEQ ID NOs: 74, 116, 150, 165, 185, and 222; SEQ ID NOs: 74, 117, 151, 180, 187, and 223; SEQ ID NOs: 88, 118, 152, 181, 194, and 224; SEQ ID NOs: 89, 119, 153, 182, 195, and 199; SEQ ID NOs: 81, 120, 154, 172, 187, and 225; SEQ ID NOs: 74, 121, 155, 165, 185, and 222; SEQ ID NOs: 85, 122, 156, 183, 196, and 226; or SEQ ID NOs: 86, 123, 157, 184, 197, and 227, respectively.

### Pharmaceutical Compositions

Provided herein are compositions comprising an anti-LY6K antibody disclosed herein having the desired degree of purity in a physiologically acceptable carrier, excipient, or stabilizer (see, e.g., Remington's Pharmaceutical Sciences (1990) Mack Publishing Co., Easton, PA). Acceptable carriers, excipients, or stabilizers are nontoxic to recipients at the dosages and concentrations employed, and include buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates, including glucose, mannose, or dextrins; chelating agents such as EDTA; sugars such as sucrose, mannitol, trehalose, or sorbitol; salt-forming counter-ions such as sodium; metal complexes (e.g., Zn-protein complexes); and/or non-ionic surfactants such as TWEEN™, PLURONICS™ or polyethylene glycol (PEG).

In a specific embodiment, pharmaceutical compositions comprise an anti-LY6K antibody disclosed herein, and optionally one or more additional prophylactic or therapeutic agents, in a pharmaceutically acceptable carrier. In a specific embodiment, pharmaceutical compositions comprise an anti-LY6K antibody disclosed herein, and optionally one or more additional prophylactic or therapeutic agents, in a pharmaceutically acceptable carrier. In certain embodiments, the antibody is the only active ingredient included in the pharmaceutical composition. Pharmaceutical compositions described herein can be useful in decreasing or blocking LY6K (e.g., human LY6K) activity and treating a condition, such as cancer. In certain embodiments, the present disclosure relates to a pharmaceutical composition of the present disclosure comprising an anti-LY6K antibody of the present disclosure for use as a medicament. In another embodiment, the present disclosure relates to a pharmaceutical composition of the present disclosure for use in a method for the treatment of cancer.

Pharmaceutically acceptable carriers used in parenteral preparations include aqueous vehicles, nonaqueous vehicles, antimicrobial agents, isotonic agents, buffers, antioxidants, local anesthetics, suspending and dispersing agents, emulsifying agents, sequestering or chelating agents, and other pharmaceutically acceptable substances. Examples of aqueous vehicles include Sodium Chloride Injection, Ringer's Injection, Isotonic Dextrose Injection, Sterile Water Injection, Dextrose and Lactated Ringer's Injection. Nonaqueous parenteral vehicles include fixed oils of vegetable origin, cottonseed oil, corn oil, sesame oil, and peanut oil. Antimicrobial agents in bacteriostatic or fungistatic concentrations can be added to parenteral preparations packaged in multiple-dose containers which include phenols or cresols, mercurials, benzyl alcohol, chlorobutanol, methyl and propyl p-hydroxybenzoic acid esters, thimerosal, benzalkonium chloride, and benzethonium chloride. Isotonic agents include sodium chloride and dextrose. Buffers include phosphate and citrate. Antioxidants include sodium bisulfate. Local anesthetics include procaine hydrochloride. Suspending and dispersing agents include sodium carboxymethylcellulose, hydroxypropyl methylcellulose, and polyvinylpyrrolidone. Emulsifying agents include Polysorbate 80 (TWEEN® 80). A sequestering or chelating agent of metal ions includes EDTA. Pharmaceutical carriers also include ethyl alcohol, polyethylene glycol, and propylene glycol for water miscible vehicles; and sodium hydroxide, hydrochloric acid, citric acid, or lactic acid for pH adjustment.

A pharmaceutical composition can be formulated for any route of administration to a subject. Specific examples of routes of administration include intranasal, oral, pulmonary, transdermal, intradermal, and parenteral. Parenteral administration, characterized by either subcutaneous, intramuscular, or intravenous injection, is also contemplated herein. Injectables can be prepared in conventional forms, either as liquid solutions or suspensions, solid forms suitable for solution or suspension in liquid prior to injection, or as emulsions. The injectables, solutions, and emulsions also contain one or more excipients. Suitable excipients are, for example, water, saline, dextrose, glycerol, or ethanol. In addition, if desired, the pharmaceutical compositions to be administered can also contain minor amounts of non-toxic auxiliary substances such as wetting or emulsifying agents, pH buffering agents, stabilizers, solubility enhancers, and other such agents, such as for example, sodium acetate, sorbitan monolaurate, triethanolamine oleate, and cyclodextrins.

Preparations for parenteral administration of antibody include sterile solutions ready for injection, sterile dry soluble products, such as lyophilized powders, ready to be combined with a solvent just prior to use, including hypodermic tablets, sterile suspensions ready for injection, sterile dry insoluble products ready to be combined with a vehicle just prior to use, and sterile emulsions. The solutions may be either aqueous or nonaqueous.

If administered intravenously, suitable carriers include physiological saline or phosphate buffered saline (PBS), and solutions containing thickening and solubilizing agents, such as glucose, polyethylene glycol, and polypropylene glycol, and mixtures thereof.

Topical mixtures comprising an antibody are prepared as described for the local and systemic administration. The resulting mixture can be a solution, suspension, emulsions, or the like and can be formulated as creams, gels, ointments, emulsions, solutions, elixirs, lotions, suspensions, tinctures, pastes, foams, aerosols, irrigations, sprays, suppositories, bandages, dermal patches, or any other formulations suitable for topical administration.

An anti-LY6K antibody disclosed herein can be formulated as an aerosol for topical application, such as by inhalation (see, e.g., U.S. Pat. Nos. 4,044,126, 4,414,209, and 4,364,923, which describe aerosols for delivery of a steroid useful for treatment of inflammatory diseases, particularly asthma and are herein incorporated by reference in their entireties). These formulations for administration to the respiratory tract can be in the form of an aerosol or solution for a nebulizer, or as a microfine powder for insufflations, alone or in combination with an inert carrier such as lactose. In such a case, the particles of the formulation will, in certain embodiments, have diameters of less than 50 microns, in certain embodiments, less than 10 microns.

An anti-LY6K antibody disclosed herein can be formulated for local or topical application, such as for topical application to the skin and mucous membranes, such as in the eye, in the form of gels, creams, and lotions and for application to the eye or for intracisternal or intraspinal application. Topical administration is contemplated for transdermal delivery and also for administration to the eyes or mucosa, or for inhalation therapies. Nasal solutions of the antibody alone or in combination with other pharmaceutically acceptable excipients can also be administered.

Transdermal patches, including iontophoretic and electrophoretic devices, are well known to those of skill in the art, and can be used to administer an antibody. For example, such patches are disclosed in U.S. Pat. Nos. 6,267,983, 6,261,595, 6,256,533, 6,167,301, 6,024,975, 6,010715, 5,985,317, 5,983,134, 5,948,433, and 5,860,957, all of which are herein incorporated by reference in their entireties.

In certain embodiments, a pharmaceutical composition comprising an antibody described herein is a lyophilized powder, which can be reconstituted for administration as solutions, emulsions, and other mixtures. It may also be reconstituted and formulated as solids or gels. The lyophilized powder is prepared by dissolving an antibody described herein, or a pharmaceutically acceptable derivative thereof, in a suitable solvent. In certain embodiments, the lyophilized powder is sterile. The solvent may contain an excipient which improves the stability or other pharmacological component of the powder or reconstituted solution, prepared from the powder. Excipients that may be used include, but are not limited to, dextrose, sorbitol, fructose, corn syrup, xylitol, glycerin, glucose, sucrose, or other suitable agent. The solvent may also contain a buffer, such as citrate, sodium or potassium phosphate, or other such buffer known to those of skill in the art, at, in certain embodiments, about neutral pH. Subsequent sterile filtration of the solution followed by lyophilization under standard conditions known to those of skill in the art provides the desired formulation. In certain embodiments, the resulting solution will be apportioned into vials for lyophilization. Each vial will contain a single dosage or multiple dosages of the compound. The lyophilized powder can be stored under appropriate conditions, such as at about 4° C. to room temperature. Reconstitution of this lyophilized powder with water for injection provides a formulation for use in parenteral administration. For reconstitution, the lyophilized powder is added to sterile water or other suitable carrier. The precise amount depends upon the selected compound. Such an amount can be empirically determined.

The anti-LY6K antibodies disclosed herein and other compositions provided herein can also be formulated to be targeted to a particular tissue, receptor, or other area of the body of the subject to be treated. Many such targeting methods are well known to those of skill in the art. All such targeting methods are contemplated herein for use in the instant compositions. For non-limiting examples of targeting methods, see, e.g., U.S. Pat. Nos. 6,316,652, 6,274,552, 6,271,359, 6,253,872, 6,139,865, 6,131,570, 6,120,751, 6,071,495, 6,060,082, 6,048,736, 6,039,975, 6,004,534, 5,985,307, 5,972,366, 5,900,252, 5,840,674, 5,759,542, and 5,709,874, all of which are herein incorporated by reference in their entireties. In a specific embodiment, an antibody described herein is targeted to a tumor.

The compositions to be used for in vivo administration can be sterile. This is readily accomplished by filtration through, e.g., sterile filtration membranes.

### Methods of Use and Uses

In another aspect, the instant disclosure provides a method of treating a subject using the anti-LY6K antibodies disclosed herein. Any disease or disorder in a subject that is characterized by overexpression of LY6K (e.g., human LY6K) can be treated using the anti-LY6K antibodies disclosed herein. In certain embodiments, the disease or disorder is resistant to a checkpoint targeting agent (e.g., an antagonist anti-CTLA-4 antibody, an antagonist anti-PD-L1 antibody, an antagonist anti-PD-L2 antibody, or an antagonist anti-PD-1 antibody). In certain embodiments, the disease or disorder is recurrent after treatment with a checkpoint targeting agent (e.g., an antagonist anti-CTLA-4 antibody, an antagonist anti-PD-L1 antibody, an antagonist anti-PD-L2 antibody, or an antagonist anti-PD-1 antibody).

In certain embodiments, the instant disclosure provides a method of treating cancer in a subject, the method comprising administering to the subject an effective amount of the antibody or pharmaceutical composition, as disclosed herein.

Cancers that can be treated with the anti-LY6K antibodies or pharmaceutical compositions disclosed herein include, without limitation, a solid tumor, a hematological cancer (e.g., leukemia, lymphoma, myeloma, e.g., multiple myeloma), and a metastatic lesion. In certain embodiments, the cancer is a solid tumor. Examples of solid tumors include malignancies, e.g., sarcomas and carcinomas, e.g., adenocarcinomas of the various organ systems, such as those affecting the lung, breast, ovarian, lymphoid, gastrointestinal (e.g., colon), anal, genitals and genitourinary tract (e.g., renal, urothelial, bladder cells, prostate), pharynx, CNS (e.g., brain, neural or glial cells), head and neck, skin (e.g., melanoma), and pancreas, as well as adenocarcinomas which include malignancies such as colon cancers, rectal cancer, renal-cell carcinoma, liver cancer, lung cancer (e.g., non-small cell lung cancer or small cell lung cancer), cancer of the small intestine, and cancer of the esophagus. The cancer may be at an early, intermediate, late stage, or metastatic cancer. In certain embodiments, the cancer is resistant to a checkpoint targeting agent (e.g., an antagonist anti-CTLA-4 antibody, an antagonist anti-PD-L1 antibody, an antagonist anti-PD-L2 antibody, or an antagonist anti-PD-1 antibody). In certain embodiments, the cancer is recurrent after treatment with a checkpoint targeting agent (e.g., an antagonist anti-CTLA-4 antibody, an antagonist anti-PD-L1 antibody, an antagonist anti-PD-L2 antibody, or an antagonist anti-PD-1 antibody).

In certain embodiments, the cancer is chosen from lung cancer (e.g., lung adenocarcinoma or non-small cell lung cancer (NSCLC) (e.g., NSCLC with squamous and/or non-squamous histology, or NSCLC adenocarcinoma)), melanoma (e.g., an advanced melanoma), renal cancer (e.g., a renal cell carcinoma), liver cancer (e.g., hepatocellular carcinoma), myeloma (e.g., a multiple myeloma), a prostate cancer, a breast cancer (e.g., a breast cancer that does not express one, two or all of estrogen receptor, progesterone receptor, or Her2/neu, e.g., a triple negative breast cancer), an ovarian cancer, a colorectal cancer, a pancreatic cancer, a head and neck cancer (e.g., head and neck squamous cell carcinoma (HNSCC)), anal cancer, gastro-esophageal cancer (e.g., esophageal squamous cell carcinoma), mesothelioma, nasopharyngeal cancer, thyroid cancer, cervical cancer, epithelial cancer, peritoneal cancer, or a lymphoproliferative disease (e.g., a post-transplant lymphoproliferative disease).

In certain embodiments, the cancer is a hematological cancer, for example, a leukemia, a lymphoma, or a myeloma. In certain embodiments, the cancer is a leukemia, for example, acute lymphoblastic leukemia (ALL), acute myelogenous leukemia (AML), acute myeloblastic leukemia (AML), chronic lymphocytic leukemia (CLL), chronic myelogenous leukemia (CML), chronic myeloid leukemia (CML), chronic myelomonocytic leukemia (CMML), chronic lymphocytic leukemia (CLL), or hairy cell leukemia. In certain embodiments, the cancer is a lymphoma, for example, B cell lymphoma, diffuse large B-cell lymphoma (DLBCL), activated B-cell like (ABC) diffuse large B cell lymphoma, germinal center B cell (GCB) diffuse large B cell lymphoma, mantle cell lymphoma, Hodgkin lymphoma, non-Hodgkin lymphoma, relapsed non-Hodgkin lymphoma, refractory non-Hodgkin lymphoma, recurrent follicular non-Hodgkin lymphoma, Burkitt lymphoma, small lymphocytic lymphoma, follicular lymphoma, lymphoplasmacytic lymphoma, or extranodal marginal zone lymphoma. In certain embodiments the cancer is a myeloma, for example, multiple myeloma.

In another embodiment, the cancer is chosen from a carcinoma (e.g., advanced or metastatic carcinoma), melanoma or a lung carcinoma, e.g., a non-small cell lung carcinoma.

In certain embodiments, the cancer is a lung cancer, e.g., a lung adenocarcinoma, non-small cell lung cancer, or small cell lung cancer.

In certain embodiments, the cancer is a melanoma, e.g., an advanced melanoma. In certain embodiments, the cancer is an advanced or unresectable melanoma that does not respond to other therapies. In other embodiments, the cancer is a melanoma with a BRAF mutation (e.g., a BRAF V600 mutation). In yet other embodiments, the anti-LY6K antibodies or pharmaceutical composition disclosed herein is administered after treatment with an anti-CTLA-4 antibody (e.g., ipilimumab) with or without a BRAF inhibitor (e.g., vemurafenib or dabrafenib).

In another embodiment, the cancer is a hepatocarcinoma, e.g., an advanced hepatocarcinoma, with or without a viral infection, e.g., a chronic viral hepatitis.

In another embodiment, the cancer is a prostate cancer, e.g., an advanced prostate cancer.

In yet another embodiment, the cancer is a myeloma, e.g., multiple myeloma.

In yet another embodiment, the cancer is a renal cancer, e.g., a renal cell carcinoma (RCC) (e.g., a metastatic RCC, clear cell renal cell carcinoma (CCRCC) or kidney papillary cell carcinoma).

In yet another embodiment, the cancer is chosen from a lung cancer, a melanoma, a renal cancer, a breast cancer, a colorectal cancer, a leukemia, or a metastatic lesion of the cancer.

In certain embodiments, these methods further comprise administering an additional therapeutic agent to the subject. In certain embodiments, the additional therapeutic agent is a chemotherapeutic, a radiotherapeutic, or a checkpoint targeting agent. In certain embodiments, the chemotherapeutic agent is a hypomethylating agent (e.g., azacitidine). In certain embodiments, the chemotherapeutic agent is a DNA damage-inducing agent (e.g., gemcitabine). In certain embodiments, the checkpoint targeting agent is selected from the group consisting of an antagonist anti-CTLA-4 antibody, an antagonist anti-PD-L1 antibody, an antagonist anti-PD-L2 antibody, an antagonist anti-PD-1 antibody, an antagonist anti-TIM-3 antibody, an antagonist anti-LAG-3 antibody, an antagonist anti-VISTA antibody, an antagonist anti-CD96 antibody, an antagonist anti-CEACAM1 antibody, an agonist anti-CD137 antibody, an agonist anti-GITR antibody, and an agonist anti-OX40 antibody. In certain embodiments, the checkpoint targeting agent is selected from the group consisting of an antagonist anti-CTLA-4 antibody, an antagonist anti-PD-L1 antibody, an antagonist anti-PD-L2 antibody, and an antagonist anti-PD-1 antibody, wherein the LY6K (e.g., human LY6K) antibodies or pharmaceutical compositions disclosed herein synergize with the checkpoint targeting agent.

In certain embodiments, the present disclosure relates to an antibody and/or pharmaceutical composition of the present disclosure for use in a method of the present disclosure, wherein the method further comprises administering an additional therapeutic agent to the subject. In certain embodiments, the present disclosure relates to (a) an antibody and/or pharmaceutical composition of the present disclosure and (b) an additional therapeutic agent for use as a medicament. In certain embodiments, the present disclosure relates to (a) an antibody and/or pharmaceutical composition of the present disclosure and (b) an additional therapeutic agent for use in a method for the treatment of cancer. In a further embodiment, the present disclosure relates to a pharmaceutical composition, kit or kit-of-parts comprising (a) an antibody and/or pharmaceutical composition of the present disclosure and (b) an additional therapeutic agent. In certain embodiments, the additional therapeutic agent is a chemotherapeutic, a radiotherapeutic, or a checkpoint targeting agent.

In certain embodiments, an anti-PD-1 antibody is used in methods disclosed herein. In certain embodiments, the anti-PD-1 antibody is nivolumab, also known as BMS-936558 or MDX1106, developed by Bristol-Myers Squibb. In certain embodiments, the anti-PD-1 antibody is pembrolizumab, also known as lambrolizumab or MK-3475, developed by Merck & Co. In certain embodiments, the anti-PD-1 antibody is pidilizumab, also known as CT-011, developed by CureTech. In certain embodiments, the anti-PD-1 antibody is MEDI0680, also known as AMP-514, developed by MedImmune. In certain embodiments, the anti-PD-1 antibody is PDR001 developed by Novartis Pharmaceuticals. In certain embodiments, the anti-PD-1 antibody is REGN2810 developed by Regeneron Pharmaceuticals. In certain embodiments, the anti-PD-1 antibody is PF-06801591 developed by Pfizer. In certain embodiments, the anti-PD-1 antibody is BGB-A317 developed by BeiGene. In certain embodiments, the anti-PD-1 antibody is TSR-042 developed by AnaptysBio and Tesaro. In certain embodiments, the anti-PD-1 antibody is SHR-1210 developed by Hengrui.

Further non-limiting examples of anti-PD-1 antibodies that may be used in treatment methods disclosed herein are disclosed in the following patents and patent applications, all of which are herein incorporated by reference in their entireties for all purposes: U.S. Pat. Nos. 6,808,710; 7,332,582; 7,488,802; 8,008,449; 8,114,845; 8,168,757; 8,354,509; 8,686,119; 8,735,553; 8,747,847; 8,779,105; 8,927,697; 8,993,731; 9,102,727; 9,205,148; U.S. Publication No. US 2013/0202623 A1; U.S. Publication No. US 2013/0291136 A1; U.S. Publication No. US 2014/0044738 A1; U.S. Publication No. US 2014/0356363 A1; U.S. Publication No. US 2016/0075783 A1; PCT Publication No. WO 2013/033091 A1; PCT Publication No. WO 2015/036394 A1; PCT Publication No. WO 2014/179664 A2; PCT Publication No. WO 2014/209804 A1; PCT Publication No. WO 2014/206107 A1; PCT Publication No. WO 2015/058573 A1; PCT Publication No. WO 2015/085847 A1; PCT Publication No. WO 2015/200119 A1; PCT Publication No. WO 2016/015685 A1; and PCT Publication No. WO 2016/020856 A1.

In certain embodiments, an anti-PD-L1 antibody is used in methods disclosed herein. In certain embodiments, the anti-PD-L1 antibody is atezolizumab developed by Genentech. In certain embodiments, the anti-PD-L1 antibody is durvalumab developed by AstraZeneca, Celgene, and MedImmune. In certain embodiments, the anti-PD-L1 antibody is avelumab, also known as MSB0010718C, developed by Merck Serono and Pfizer. In certain embodiments, the anti-PD-L1 antibody is MDX-1105 developed by Bristol-Myers Squibb. In certain embodiments, the anti-PD-L1 antibody is AMP-224 developed by Amplimmune and GSK.

Non-limiting examples of anti-PD-L1 antibodies that may be used in treatment methods disclosed herein are disclosed in the following patents and patent applications, all of which are herein incorporated by reference in their entireties for all purposes: U.S. Pat. Nos. 7,943,743; 8,168,179; 8,217,149; 8,552,154; 8,779,108; 8,981,063; 9,175,082; U.S. Publication No. US 2010/0203056 A1; U.S. Publication No. US 2003/0232323 A1; U.S. Publication No. US 2013/0323249 A1; U.S. Publication No. US 2014/0341917 A1; U.S. Publication No. US 2014/0044738 A1; U.S. Publication No. US 2015/0203580 A1; U.S. Publication No. US 2015/0225483 A1; U.S. Publication No. US 2015/0346208 A1; U.S. Publication No. US 2015/0355184 A1; PCT Publication No. WO 2014/100079 A1; PCT Publication No. WO 2014/022758 A1; PCT Publication No. WO 2014/055897 A2; PCT Publication No. WO 2015/061668 A1; PCT Publication No. WO 2015/109124 A1; PCT Publication No. WO 2015/195163 A1; PCT Publication No. WO 2016/000619 A1; and PCT Publication No. WO 2016/030350 A1.

In certain embodiments, an anti-CTLA-4 antibody is used in methods disclosed herein. In certain embodiments, the anti-CTLA-4 antibody is ipilimumab developed by Bristol-Myers Squibb.

In certain embodiments, an anti-LY6K antibody disclosed herein is administered to a subject in combination with a compound that targets an immunomodulatory enzyme(s) such as IDO (indoleamine-(2,3)-dioxygenase) and/or TDO (tryptophan 2,3-dioxygenase). Therefore, in certain embodiments, the additional therapeutic agent is a compound that targets an immunomodulatory enzyme(s), such as an inhibitor of indoleamine-(2,3)-dioxygenase (IDO). In certain embodiments, such compound is selected from the group consisting of epacadostat (Incyte Corp; see, e.g., WO 2010/005958 which is herein incorporated by reference in its entirety), F001287 (Flexus Biosciences/Bristol-Myers Squibb), indoximod (NewLink Genetics), and NLG919 (NewLink Genetics). In certain embodiments, the compound is epacadostat. In another embodiment, the compound is F001287. In another embodiment, the compound is indoximod. In another embodiment, the compound is NLG919. In a specific embodiment, an anti-LY6K antibody disclosed herein is administered to a subject in combination with an IDO inhibitor for treating cancer. The IDO inhibitor as described herein for use in treating cancer is present in a solid dosage form of a pharmaceutical composition such as a tablet, a pill, or a capsule, wherein the pharmaceutical composition includes an IDO inhibitor and a pharmaceutically acceptable excipient. As such, the antibody as described herein and the IDO inhibitor as described herein can be administered separately, sequentially, or concurrently as separate dosage forms. In certain embodiments, the antibody is administered parenterally, and the IDO inhibitor is administered orally. In certain embodiments, the inhibitor is selected from the group consisting of epacadostat (Incyte Corporation), F001287 (Flexus Biosciences/Bristol-Myers Squibb), indoximod (NewLink Genetics), and NLG919 (NewLink Genetics). Epacadostat has been described in PCT Publication No. WO 2010/005958, which is herein incorporated by reference in its entirety for all purposes. In certain embodiments, the inhibitor is epacadostat. In another embodiment, the inhibitor is F001287. In another embodiment, the inhibitor is indoximod. In another embodiment, the inhibitor is NLG919.

In certain embodiments, an anti-LY6K antibody disclosed herein is administered to a subject in combination with a vaccine. The vaccine can be, e.g., a peptide vaccine, a DNA vaccine, or an RNA vaccine.

In certain embodiments, an anti-LY6K antibody disclosed herein is administered to a subject in combination with an adjuvant. Various adjuvants can be used depending on the treatment context. Non-limiting examples of appropriate adjuvants include, but not limited to, Complete Freund's Adjuvant (CFA), Incomplete Freund's Adjuvant (IFA), montanide ISA (incomplete Seppic adjuvant), the Ribi adjuvant system (RAS), Titer Max, muramyl peptides, Syntex Adjuvant Formulation (SAF), alum (aluminum hydroxide and/or aluminum phosphate), aluminum salt adjuvants, Gerbu® adjuvants, nitrocellulose absorbed antigen, encapsulated or entrapped antigen, 3 De-O-acylated monophosphoryl lipid A (3 D-MPL), immunostimulatory oligonucleotides, toll-like receptor (TLR) ligands, mannan-binding lectin (MBL) ligands, STING agonists, immuno-stimulating complexes such as saponins, Quil A, QS-21, QS-7, ISCOMATRIX, and others. Other adjuvants include CpG oligonucleotides and double stranded RNA molecules, such as poly(A) and poly(U). Combinations of the above adjuvants may also be used. See, e.g., U.S. Pat. Nos. 6,645,495; 7,029,678; and 7,858,589, all of which are incorporated herein by reference in their entireties. In certain embodiments, the adjuvant used herein is QS-21 STIMULON.

In certain embodiments, an anti-LY6K antibody disclosed herein is administered to a subject in combination with an additional therapeutic agent comprising a TCR. In certain embodiments, the additional therapeutic agent is a soluble TCR. In certain embodiments, the additional therapeutic agent is a cell expressing a TCR. Therefore, in certain embodiments, the present disclosure relates to an antibody and/or pharmaceutical composition of the present disclosure in combination with an additional therapeutic agent comprising a TCR for use as a medicament and/or for use in a method for the treatment of cancer.

In certain embodiments, an anti-LY6K antibody disclosed herein is administered to a subject in combination with a cell expressing a chimeric antigen receptor (CAR). In certain embodiments, the cell is a T cell.

In certain embodiments, an anti-LY6K antibody disclosed herein is administered to a subject in combination with a TCR mimic antibody. In certain embodiments, the TCR mimic antibody is an antibody that specifically binds to a peptide-MHC complex. For non-limiting examples of TCR mimic antibodies, see, e.g., U.S. Pat. No. 9,074,000 and U.S. Publication Nos. US 2009/0304679 A1 and US 2014/0134191 A1, all of which are incorporated herein by reference in their entireties.

In certain embodiments, an anti-LY6K antibody disclosed herein is administered to a subject in combination with a bispecific T-cell engager (BiTE) (e.g., as described in WO2005061547A2, which is incorporated by reference herein in its entirety) and/or a dual-affinity re-targeting antibody (DART) (e.g., as described in WO2012162067A2, which is incorporated by reference herein in its entirety). In certain embodiments, the BiTE and/or DART specifically binds to a tumor-associated antigen (e.g., a polypeptide overexpressed in a tumor, a polypeptide derived from an oncovirus, a polypeptide comprising a post-translational modification specific to a tumor, a polypeptide specifically mutated in a tumor) and a molecule on an effector cell (e.g., CD3 or CD16). In certain embodiments, the tumor-associated antigen is EGFR (e.g., human EGFR), optionally wherein the BiTE and/or DART comprises the VH and VL sequences of cetuximab. In certain embodiments, the tumor-associated antigen is Her2 (e.g., human Her2), optionally wherein the BiTE and/or DART comprises the VH and VL sequences of trastuzumab. In certain embodiments, the tumor-associated antigen is CD20 (e.g., human CD20).

The anti-LY6K antibody and the additional therapeutic agent (e.g., chemotherapeutic, radiotherapeutic, checkpoint targeting agent, IDO inhibitor, vaccine, adjuvant, a soluble TCR, a cell expressing a TCR, a cell expressing a chimeric antigen receptor, and/or a TCR mimic antibody) can be administered separately, sequentially, or concurrently as separate dosage forms. In certain embodiments, an anti-LY6K antibody is administered parenterally, and an IDO inhibitor is administered orally.

An antibody or pharmaceutical composition described herein may be delivered to a subject by a variety of routes. These include, but are not limited to, parenteral, intranasal, intratracheal, oral, intradermal, topical, intramuscular, intraperitoneal, transdermal, intravenous, intratumoral, conjunctival, intra-arterial, and subcutaneous routes. Pulmonary administration can also be employed, e.g., by use of an inhaler or nebulizer, and formulation with an aerosolizing agent for use as a spray. In certain embodiments, the antibody or pharmaceutical composition described herein is delivered subcutaneously or intravenously. In certain embodiments, the antibody or pharmaceutical composition described herein is delivered intra-arterially. In certain embodiments, the antibody or pharmaceutical composition described herein is delivered intratumorally. In certain embodiments, the antibody or pharmaceutical composition described herein is delivered into a tumor draining lymph node.

The amount of an antibody or composition which will be effective in the treatment and/or prevention of a condition will depend on the nature of the disease and can be determined by standard clinical techniques.

The precise dose to be employed in a composition will also depend on the route of administration, and the seriousness of the infection or disease caused by it and should be decided according to the judgment of the practitioner and each subject's circumstances. For example, effective doses may also vary depending upon means of administration, target site, physiological state of the patient (including age, body weight, and health), whether the patient is human or an animal, other medications administered, or whether treatment is prophylactic or therapeutic. Usually, the patient is a human, but non-human mammals, including transgenic mammals, can also be treated. Treatment dosages are optimally titrated to optimize safety and efficacy.

An anti-LY6K antibody described herein can also be used to assay LY6K (e.g., human LY6K) protein levels in a biological sample using classical immunohistological methods known to those of skill in the art, including immunoassays, such as the enzyme linked immunosorbent assay (ELISA), immunoprecipitation, or Western blotting. Suitable antibody assay labels are known in the art and include enzyme labels, such as, glucose oxidase; radioisotopes, such as iodine (¹²¹, ¹²¹I), carbon (C), sulfur (³⁵S), tritium (H), indium (¹²¹In), and technetium (⁹⁹Tc); luminescent labels, such as luminol; and fluorescent labels, such as fluorescein and rhodamine, and biotin. Such labels can be used to label an antibody described herein. Alternatively, a second antibody that recognizes an anti-LY6K antibody described herein can be labeled and used in combination with an anti-LY6K antibody to detect LY6K (e.g., human LY6K) protein levels. Therefore, in certain embodiments, the present disclosure relates to the use of an anti-LY6K antibody of the present disclosure for in vitro detection of LY6K (e.g., human LY6K) protein in a biological sample. In a further embodiment, the present disclosure relates to the use of an anti-LY6K antibody of the disclosure, for assaying and/or detecting LY6K (e.g., human LY6K) protein levels in a biological sample in vitro, optionally wherein the anti-LY6K antibody is conjugated to a radionuclide or detectable label, and/or carries a label described herein, and/or wherein an immunohistological method is used.

Assaying for the expression level of LY6K (e.g., human LY6K) protein is intended to include qualitatively or quantitatively measuring or estimating the level of LY6K (e.g., human LY6K) protein in a first biological sample either directly (e.g., by determining or estimating absolute protein level) or relatively (e.g., by comparing to the disease associated protein level in a second biological sample). LY6K (e.g., human LY6K) polypeptide expression level in the first biological sample can be measured or estimated and compared to a standard LY6K (e.g., human LY6K) protein level, the standard being taken, for example, from a second biological sample obtained from an individual not having the disorder or being determined by averaging levels from a population of individuals not having the disorder. As will be appreciated in the art, once the “standard” LY6K (e.g., human LY6K) polypeptide level is known, it can be used repeatedly as a standard for comparison. Therefore, in a further embodiment, the present disclosure relates to an in vitro method for assaying and/or detecting LY6K protein levels, for example human LY6K protein levels, in a biological sample, comprising qualitatively or quantitatively measuring or estimating the level of LY6K protein, for example of human LY6K protein, in a biological sample, by an immunohistological method.

As used herein, the term “biological sample” refers to any biological sample obtained from a subject, cell line, tissue, or other source of cells potentially expressing LY6K (e.g., human LY6K). Methods for obtaining tissue biopsies and body fluids from animals (e.g., humans or cynomolgus monkeys) are well known in the art. Biological samples include peripheral blood mononuclear cells (PBMCs).

An anti-LY6K antibody described herein can be used for prognostic, diagnostic, monitoring, and screening applications, including in vitro and in vivo applications well known and standard to the skilled artisan and based on the present description. Prognostic, diagnostic, monitoring, and screening assays and kits for in vitro assessment and evaluation of immune system status, immune response, and/or cancer (e.g., tumorigenesis) may be utilized to predict, diagnose, and monitor to evaluate patient samples, including those known to have or suspected of having an immune system-dysfunction or cancer, or with regard to an anticipated or desired immune system response, antigen response, or vaccine response. The assessment and evaluation of immune system status, immune response, and/or cancer are also useful in determining the suitability of a patient for a clinical trial of a drug or for the administration of a particular chemotherapeutic agent, a radiotherapeutic agent, or an antibody, including combinations thereof, versus a different agent or antibody. This type of prognostic and diagnostic monitoring and assessment is already in practice utilizing antibodies against the HER2 protein in breast cancer (HercepTest™, Dako) where the assay is also used to evaluate patients for antibody therapy using Herceptin®. In vivo applications include directed cell therapy and immune system modulation and radio imaging of immune responses. Therefore, in certain embodiments, the present disclosure relates to an anti-LY6K antibody and/or pharmaceutical composition of the present disclosure for use as a diagnostic. In certain embodiments, the present disclosure relates to an anti-LY6K antibody and/or pharmaceutical composition of the present disclosure for use in a method for the prediction, diagnosis, and/or monitoring of a subject having or suspected to have an immune system-dysfunction, cancer, and/or with regard to an anticipated or desired immune system response, antigen response, or vaccine response. In another embodiment, the present disclosure relates to the use of an anti-LY6K antibody of the disclosure, for predicting, diagnosing, and/or monitoring of a subject having or suspected to have an immune system-dysfunction, cancer, and/or with regard to an anticipated or desired immune system response, antigen response, or vaccine response by assaying and/or detecting human LY6K protein levels in a biological sample of the subject in vitro.

In certain embodiments, an anti-LY6K antibody can be used in immunohistochemistry of biopsy samples. In certain embodiments, the method is an in vitro method. In another embodiment, an anti-LY6K antibody can be used to detect levels of LY6K (e.g., human LY6K), or levels of cells which contain LY6K (e.g., human LY6K) on their membrane surface, the levels of which can then be linked to certain disease symptoms. Anti-LY6K antibodies described herein may carry a detectable or functional label and/or may be conjugated to a radionuclide or detectable label. When fluorescence labels are used, currently available microscopy and fluorescence-activated cell sorter analysis (FACS) or combination of both methods procedures known in the art may be utilized to identify and to quantitate the specific binding members. Anti-LY6K antibodies described herein may carry or may be conjugated to a fluorescence label. Exemplary fluorescence labels include, for example, reactive and conjugated probes, e.g., Aminocoumarin, Fluorescein and Texas red, Alexa Fluor dyes, Cy dyes, and DyLight dyes. An anti-LY6K antibody may carry or may be conjugated to a radioactive label or radionuclide, such as the isotopes ³H, ¹⁴C, ³²P, ³⁵S, ³⁶Cl, ⁵¹Cr, ⁵⁷Co, ⁵⁸Co, ⁵⁹Fe, ⁶⁷Cu, ⁹⁰Y, ⁹⁹Tc, ¹¹¹In, ¹¹⁷Lu, ¹²¹I, ¹²⁴I, ¹²⁵I, ¹³¹I, ¹⁹⁸Au, ²¹¹At, ²¹³Bi, ²²⁵Ac, and ¹⁸⁶Re. When radioactive labels are used, currently available counting procedures known in the art may be utilized to identify and quantitate the specific binding of an anti-LY6K antibody to LY6K (e.g., human LY6K). In the instance where the label is an enzyme, detection may be accomplished by any of the presently utilized colorimetric, spectrophotometric, fluorospectrophotometric, amperometric, or gasometric techniques as known in the art. This can be achieved by contacting a sample or a control sample with an anti-LY6K antibody under conditions that allow for the formation of a complex between the anti-LY6K antibody and LY6K (e.g., human LY6K). Any complexes formed between the anti-LY6K antibody and LY6K (e.g., human LY6K), are detected and compared in the sample and the control. In light of the specific binding of the anti-LY6K antibodies described herein for LY6K (e.g., human LY6K), the anti-LY6K antibodies can be used to specifically detect LY6K (e.g., human LY6K). The anti-LY6K antibodies described herein can also be used to purify LY6K (e.g., human LY6K) via immunoaffinity purification. Also included herein is an assay system which may be prepared in the form of a test kit, kit, or kit-of-parts for the quantitative analysis of the extent of the presence of, for instance, LY6K (e.g., human LY6K)/LY6K (e.g., human LY6K) ligand complexes. The system, test kit, kit, or kit-of-parts may comprise a labeled component, e.g., a labeled antibody, and one or more additional immunochemical reagents.

### Polynucleotides, Vectors, and Methods of Producing Antibodies

In another aspect, provided herein are polynucleotides comprising a nucleotide sequence encoding an antibody, or a portion thereof, described herein or a fragment thereof (e.g., a VL and/or VH; and a light chain and/or heavy chain) that specifically binds to a LY6K (e.g., human LY6K) antigen, and vectors, e.g., vectors comprising such polynucleotides for recombinant expression in host cells (e.g., E. coli and mammalian cells). Provided herein are polynucleotides comprising nucleotide sequences encoding a heavy and/or light chain of any of the antibodies provided herein, as well as vectors comprising such polynucleotide sequences, e.g., expression vectors for their efficient expression in host cells, e.g., mammalian cells. In certain embodiments, a nucleic acid molecule(s) encoding an antibody described herein is isolated or purified.

In particular aspects, provided herein are polynucleotides comprising nucleotide sequences encoding antibodies, which specifically bind to a LY6K (e.g., human LY6K) polypeptide and comprise an amino acid sequence as described herein, as well as antibodies which compete with such antibodies for binding to a LY6K (e.g., human LY6K) polypeptide (e.g., in a dose-dependent manner), or which binds to the same epitope as that of such antibodies.

In certain aspects, provided herein are polynucleotides comprising a nucleotide sequence encoding the light chain or heavy chain of an antibody described herein. The polynucleotides can comprise nucleotide sequences encoding a light chain comprising the VL FRs and CDRs of antibodies described herein (see, e.g., Tables 2 and 4) or nucleotide sequences encoding a heavy chain comprising the VH FRs and CDRs of antibodies described herein (see, e.g., Tables 1 and 3). In certain embodiments, a polynucleotide encodes a VH, VL, heavy chain, and/or light chain of an antibody described herein. In another embodiment, a polynucleotide encodes the first VH and the first VL of an antibody described herein. In another embodiment, a polynucleotide encodes the second VH and the second VL of an antibody described herein. In another embodiment, a polynucleotide encodes the first heavy chain and the first light chain of an antibody described herein. In another embodiment, a polynucleotide encodes the second heavy chain and the second light chain of an antibody described herein. In another embodiment, a polynucleotide encodes the VH and/or the VL, or the heavy chain and/or the light chain, of an antibody described herein.

Also provided herein are polynucleotides encoding an anti-LY6K antibody that are optimized, e.g., by codon/RNA optimization, replacement with heterologous signal sequences, and elimination of mRNA instability elements. Methods to generate optimized nucleic acids encoding an anti-LY6K antibody or a fragment thereof (e.g., light chain, heavy chain, VH domain, or VL domain) for recombinant expression by introducing codon changes and/or eliminating inhibitory regions in the mRNA can be carried out by adapting the optimization methods described in, e.g., U.S. Pat. Nos. 5,965,726; 6,174,666; 6,291,664; 6,414,132; and 6,794,498, accordingly, all of which are herein incorporated by reference in their entireties. For example, potential splice sites and instability elements (e.g., A/T or A/U rich elements) within the RNA can be mutated without altering the amino acids encoded by the nucleic acid sequences to increase stability of the RNA for recombinant expression. The alterations utilize the degeneracy of the genetic code, e.g., using an alternative codon for an identical amino acid. In certain embodiments, it can be desirable to alter one or more codons to encode a conservative mutation, e.g., a similar amino acid with similar chemical structure and properties and/or function as the original amino acid. Such methods can increase expression of an anti-LY6K antibody or fragment thereof by at least 1 fold, 2 fold, 3 fold, 4 fold, 5 fold, 10 fold, 20 fold, 30 fold, 40 fold, 50 fold, 60 fold, 70 fold, 80 fold, 90 fold, or 100 fold or more relative to the expression of an anti-LY6K antibody encoded by polynucleotides that have not been optimized.

In certain embodiments, an optimized polynucleotide sequence encoding an anti-LY6K antibody described herein or a fragment thereof (e.g., VL domain and/or VH domain) can hybridize to an antisense (e.g., complementary) polynucleotide of an unoptimized polynucleotide sequence encoding an anti-LY6K antibody described herein or a fragment thereof (e.g., VL domain and/or VH domain). In specific embodiments, an optimized nucleotide sequence encoding an anti-LY6K antibody described herein or a fragment thereof hybridizes under high stringency conditions to antisense polynucleotide of an unoptimized polynucleotide sequence encoding an anti-LY6K antibody described herein or a fragment thereof. In a specific embodiment, an optimized nucleotide sequence encoding an anti-LY6K antibody described herein or a fragment thereof hybridizes under high stringency, intermediate or lower stringency hybridization conditions to an antisense polynucleotide of an unoptimized nucleotide sequence encoding an anti-LY6K antibody described herein or a fragment thereof. Information regarding hybridization conditions has been described, see, e.g., U.S. Patent Application Publication No. US 2005/0048549 (e.g., paragraphs 72-73), which is herein incorporated by reference in its entirety.

The polynucleotides can be obtained, and the nucleotide sequence of the polynucleotides determined, by any method known in the art. Nucleotide sequences encoding antibodies described herein, e.g., antibodies described in Tables 1-4, and modified versions of these antibodies can be determined using methods known in the art, i.e., nucleotide codons known to encode particular amino acids are assembled in such a way to generate a nucleic acid that encodes the antibody. Such a polynucleotide encoding the antibody can be assembled from chemically synthesized oligonucleotides (e.g., as described in Kutmeier G et al., (1994), BioTechniques 17: 242-6, herein incorporated by reference in its entirety), which, briefly, involves the synthesis of overlapping oligonucleotides containing portions of the sequence encoding the antibody, annealing and ligating of those oligonucleotides, and then amplification of the ligated oligonucleotides by PCR.

Alternatively, a polynucleotide encoding an antigen-binding region of an antibody described herein can be generated from nucleic acid from a suitable source (e.g., a hybridoma) using methods well known in the art (e.g., PCR and other molecular cloning methods). For example, PCR amplification using synthetic primers hybridizable to the 3′ and 5′ ends of a known sequence can be performed using genomic DNA obtained from hybridoma cells producing the antibody of interest. Such PCR amplification methods can be used to obtain nucleic acids comprising the sequence encoding the light chain and/or heavy chain of an antibody. Such PCR amplification methods can be used to obtain nucleic acids comprising the sequence encoding the variable light chain region and/or the variable heavy chain region of an antibody. The amplified nucleic acids can be cloned into vectors for expression in host cells and for further cloning.

If a clone containing a nucleic acid encoding a particular antigen-binding region or antibody is not available, but the sequence of the antigen-binding region or antibody molecule is known, a nucleic acid encoding the immunoglobulin can be chemically synthesized or obtained from a suitable source (e.g., an antibody cDNA library or a cDNA library generated from, or nucleic acid, preferably poly A+ RNA, isolated from, any tissue or cells expressing the antibody, such as hybridoma cells selected to express an antibody described herein) by PCR amplification using synthetic primers hybridizable to the 3′ and 5′ ends of the sequence, or by cloning using an oligonucleotide probe specific for the particular gene sequence to identify, e.g., a cDNA clone from a cDNA library that encodes the antibody. Amplified nucleic acids generated by PCR can then be cloned into replicable cloning vectors using any method well known in the art.

DNA encoding anti-LY6K (e.g., human LY6K) antibodies described herein can be readily isolated and sequenced using conventional procedures (e.g., by using oligonucleotide probes that are capable of binding specifically to genes encoding the heavy and light chains of the anti-LY6K (e.g., human LY6K) antibodies). Hybridoma cells can serve as a source of such DNA. Once isolated, the DNA can be placed into expression vectors, which are then transfected into host cells such as E. coli cells, simian COS cells, Chinese hamster ovary (CHO) cells (e.g., CHO cells from the CHO GS System™ (Lonza)), or myeloma cells that do not otherwise produce immunoglobulin protein, to obtain the synthesis of anti-LY6K antibodies in the recombinant host cells.

To generate whole antibodies or antigen-binding regions, PCR primers including VH or VL nucleotide sequences, a restriction site, and a flanking sequence to protect the restriction site can be used to amplify the VH or VL sequences in scFv clones. Utilizing cloning techniques known to those of skill in the art, the PCR amplified VH domains can be cloned into vectors expressing a heavy chain constant region, e.g., the human gamma 1 or human gamma 4 constant region, and the PCR amplified VL domains can be cloned into vectors expressing a light chain constant region, e.g., human kappa or lambda constant regions. In certain embodiments, the vectors for expressing the VH or VL domains comprise an EF-1α promoter, a secretion signal, a cloning site for the variable region, constant regions, and a selection marker such as neomycin. The VH and VL domains can also be cloned into one vector expressing the necessary constant regions. The heavy chain conversion vectors and light chain conversion vectors are then co-transfected into cell lines to generate stable or transient cell lines that express full-length antibodies, e.g., IgG, using techniques known to those of skill in the art.

The DNA also can be modified, for example, by substituting the coding sequence for human heavy and light chain constant regions in place of the murine sequences, or by covalently joining to the immunoglobulin coding sequence all or part of the coding sequence for a non-immunoglobulin polypeptide.

Also provided are polynucleotides that hybridize under high stringency, intermediate or lower stringency hybridization conditions to polynucleotides that encode an antibody described herein. In specific embodiments, polynucleotides described herein hybridize under high stringency, intermediate or lower stringency hybridization conditions to polynucleotides encoding a VH domain and/or VL domain provided herein.

Hybridization conditions have been described in the art and are known to one of skill in the art. For example, hybridization under stringent conditions can involve hybridization to filter-bound DNA in 6× sodium chloride/sodium citrate (SSC) at about 45° C. followed by one or more washes in 0.2×SSC/0.1% SDS at about 50-65° C.; hybridization under highly stringent conditions can involve hybridization to filter-bound nucleic acid in 6×SSC at about 45° C. followed by one or more washes in 0.1×SSC/0.2% SDS at about 68° C. Hybridization under other stringent hybridization conditions are known to those of skill in the art and have been described, see, e.g., Ausubel F M et al., eds., (1989) Current Protocols in Molecular Biology, Vol. I, Green Publishing Associates, Inc. and John Wiley & Sons, Inc., New York at pages 6.3.1-6.3.6 and 2.10.3, which is herein incorporated by reference in its entirety.

In certain aspects, provided herein are cells (e.g., host cells) expressing (e.g., recombinantly) antibodies described herein which specifically bind to LY6K (e.g., human LY6K), and related polynucleotides and expression vectors. Provided herein are vectors (e.g., expression vectors) comprising polynucleotides comprising nucleotide sequences encoding anti-LY6K antibodies or a fragment for recombinant expression in host cells, preferably in mammalian cells (e.g., CHO cells). Also provided herein are host cells comprising such vectors for recombinantly expressing anti-LY6K antibodies described herein (e.g., human or humanized antibody). In a particular aspect, provided herein are methods for producing an antibody described herein, comprising expressing the antibody from a host cell.

Recombinant expression of an antibody described herein (e.g., a full-length antigen-binding region or antibody or heavy and/or light chain of an antibody described herein) that specifically binds to LY6K (e.g., human LY6K) generally involves construction of an expression vector containing a polynucleotide that encodes the antibody. Once a polynucleotide encoding an antibody molecule, heavy and/or light chain of an antibody, or a fragment thereof (e.g., heavy and/or light chain variable regions) described herein has been obtained, the vector for the production of the antibody molecule can be produced by recombinant DNA technology using techniques well known in the art. Thus, methods for preparing a protein by expressing a polynucleotide containing an antibody or antibody fragment (e.g., light chain or heavy chain) encoding nucleotide sequence are described herein. Methods which are well known to those skilled in the art can be used to construct expression vectors containing an antibody or antibody fragment (e.g., light chain or heavy chain) coding sequences and appropriate transcriptional and translational control signals. These methods include, for example, in vitro recombinant DNA techniques, synthetic techniques, and in vivo genetic recombination. Also provided are replicable vectors comprising a nucleotide sequence encoding containing an antibody molecule described herein, a heavy or light chain of an antibody, a heavy or light chain variable region of an antibody or a fragment thereof, or a heavy or light chain CDR, operably linked to a promoter. Such vectors can, for example, include the nucleotide sequence encoding the constant region of the antibody molecule (see, e.g., International Publication Nos. WO 86/05807 and WO 89/01036; and U.S. Pat. No. 5,122,464, which are herein incorporated by reference in their entireties) and variable regions of the antibody can be cloned into such a vector for expression of the entire heavy, the entire light chain, or both the entire heavy and light chains.

In certain embodiments, a vector comprises a polynucleotide encoding a VH, VL, heavy chain, and/or light chain of an antibody described herein. In another embodiment, a vector comprises a polynucleotide encoding the VH and the VL of an antibody described herein. In another embodiment, a vector comprises a polynucleotide encoding the heavy chain and the light chain of an antibody described herein.

An expression vector can be transferred to a cell (e.g., host cell) by conventional techniques and the resulting cells can then be cultured by conventional techniques to produce cells containing an antibody described herein or a fragment thereof. Thus, provided herein are host cells containing a polynucleotide encoding containing an antibody described herein or fragments thereof, or a heavy or light chain thereof, or fragment thereof, or a single-chain antibody described herein, operably linked to a promoter for expression of such sequences in the host cell.

In certain embodiments, a host cell comprises a polynucleotide encoding the VH and VL of an antibody described herein. In another embodiment, a host cell comprises a vector comprising a polynucleotide encoding the VH and VL of an antibody described herein. In another embodiment, a host cell comprises a first polynucleotide encoding the VH of an antibody described herein, and a second polynucleotide encoding the VL of an antibody described herein. In another embodiment, a host cell comprises a first vector comprising a first polynucleotide encoding the VH of an antibody described herein, and a second vector comprising a second polynucleotide encoding the VL of an antibody described herein.

In specific embodiments, a heavy chain/heavy chain variable region expressed by a first cell is associated with a light chain/light chain variable region of a second cell to form an anti-LY6K (e.g., human LY6K) antibody described herein. In certain embodiments, provided herein is a population of host cells comprising such first host cell and such second host cell.

In certain embodiments, provided herein is a population of vectors comprising a first vector comprising a polynucleotide encoding a light chain/light chain variable region of an anti-LY6K (e.g., human LY6K) antibody described herein, and a second vector comprising a polynucleotide encoding a heavy chain/heavy chain variable region of an anti-LY6K (e.g., human LY6K) antibody described herein.

A variety of host-expression vector systems can be utilized to express antibody molecules described herein (see, e.g., U.S. Pat. No. 5,807,715, which is herein incorporated by reference in its entirety). Such host-expression systems represent vehicles by which the coding sequences of interest can be produced and subsequently purified, but also represent cells which can, when transformed or transfected with the appropriate nucleotide coding sequences, express an antibody molecule described herein in situ. These include but are not limited to microorganisms such as bacteria (e.g., E. coli and B. subtilis) transformed with, e.g., recombinant bacteriophage DNA, plasmid DNA or cosmid DNA expression vectors containing antibody coding sequences; yeast (e.g., Saccharomyces and Pichia) transformed with, e.g., recombinant yeast expression vectors containing antibody coding sequences; insect cell systems infected with, e.g., recombinant virus expression vectors (e.g., baculovirus) containing antibody coding sequences; plant cell systems (e.g., green algae such as Chlamydomonas reinhardtii) infected with, e.g., recombinant virus expression vectors (e.g., cauliflower mosaic virus, CaMV; tobacco mosaic virus, TMV) or transformed with, e.g., recombinant plasmid expression vectors (e.g., Ti plasmid) containing antibody coding sequences; or mammalian cell systems (e.g., COS (e.g., COS1 or COS), CHO, BHK, MDCK, HEK 293, NS0, PER.C6, VERO, CRL7030, HsS78Bst, HeLa, and NIH 3T3, HEK-293T, HepG2, SP210, R1.1, B-W, L-M, BSC1, BSC40, YB/20 and BMT10 cells) harboring, e.g., recombinant expression constructs containing promoters derived from the genome of mammalian cells (e.g., metallothionein promoter) or from mammalian viruses (e.g., the adenovirus late promoter; the vaccinia virus 7.5K promoter). In a specific embodiment, cells for expressing antibodies described herein are Chinese hamster ovary (CHO) cells, for example CHO cells from the CHO GS System™ (Lonza). In certain embodiments, the heavy chain and/or light chain of an antibody produced by a CHO cell may have an N-terminal glutamine or glutamate residue replaced by pyroglutamate. In certain embodiments, cells for expressing antibodies described herein are human cells, e.g., human cell lines. In a specific embodiment, a mammalian expression vector is pOptiVEC™ or pcDNA3.3. In certain embodiments, bacterial cells such as Escherichia coli, or eukaryotic cells (e.g., mammalian cells), especially for the expression of whole recombinant antibody molecule, are used for the expression of a recombinant antibody molecule. For example, mammalian cells such as CHO cells, in conjunction with a vector such as the major intermediate early gene promoter element from human cytomegalovirus, are an effective expression system for antibodies (Foecking M K & Hofstetter H (1986) Gene 45: 101-5; and Cockett M I et al., (1990) Biotechnology 8(7): 662-7, each of which is herein incorporated by reference in its entirety). In certain embodiments, antibodies described herein are produced by CHO cells or NS0 cells. In a specific embodiment, the expression of nucleotide sequences encoding antibodies described herein which specifically bind to LY6K (e.g., human LY6K) is regulated by a constitutive promoter, inducible promoter, or tissue specific promoter.

In bacterial systems, a number of expression vectors can be advantageously selected depending upon the use intended for the antibody molecule being expressed. For example, when a large quantity of such an antibody is to be produced, for the generation of pharmaceutical compositions of an antibody molecule, vectors which direct the expression of high levels of fusion protein products that are readily purified can be desirable. Such vectors include, but are not limited to, the E. coli expression vector pUR278 (Ruether U & Mueller-Hill B (1983) EMBO J2: 1791-1794), in which the coding sequence can be ligated individually into the vector in frame with the lac Z coding region so that a fusion protein is produced; pIN vectors (Inouye S & Inouye M (1985) Nuc Acids Res 13: 3101-3109; Van Heeke G & Schuster S M (1989) J Biol Chem 24: 5503-5509); and the like, all of which are herein incorporated by reference in their entireties. For example, pGEX vectors can also be used to express foreign polypeptides as fusion proteins with glutathione 5-transferase (GST). In general, such fusion proteins are soluble and can easily be purified from lysed cells by adsorption and binding to matrix glutathione agarose beads followed by elution in the presence of free glutathione. The pGEX vectors are designed to include thrombin or factor Xa protease cleavage sites so that the cloned target gene product can be released from the GST moiety.

In an insect system, Autographa californica nuclear polyhedrosis virus (AcNPV), for example, can be used as a vector to express foreign genes. The virus grows in Spodoptera frugiperda cells. The coding sequence can be cloned individually into non-essential regions (for example the polyhedrin gene) of the virus and placed under control of an AcNPV promoter (for example the polyhedrin promoter).

In mammalian host cells, a number of viral-based expression systems can be utilized. In cases where an adenovirus is used as an expression vector, the coding sequence of interest can be ligated to an adenovirus transcription/translation control complex, e.g., the late promoter and tripartite leader sequence. This chimeric gene can then be inserted in the adenovirus genome by in vitro or in vivo recombination. Insertion in a non-essential region of the viral genome (e.g., region E1 or E3) will result in a recombinant virus that is viable and capable of expressing the molecule in infected hosts (see, e.g., Logan J & Shenk T (1984) PNAS 81(12): 3655-9, which is herein incorporated by reference in its entirety). Specific initiation signals can also be required for efficient translation of inserted coding sequences. These signals include the ATG initiation codon and adjacent sequences. Furthermore, the initiation codon must be in phase with the reading frame of the desired coding sequence to ensure translation of the entire insert. These exogenous translational control signals and initiation codons can be of a variety of origins, both natural and synthetic. The efficiency of expression can be enhanced by the inclusion of appropriate transcription enhancer elements, transcription terminators, etc. (see, e.g., Bitter G et al., (1987) Methods Enzymol. 153: 516-544, which is herein incorporated by reference in its entirety).

In addition, a host cell strain can be chosen which modulates the expression of the inserted sequences or modifies and processes the gene product in the specific fashion desired. Such modifications (e.g., glycosylation) and processing (e.g., cleavage) of protein products can be important for the function of the protein. Different host cells have characteristic and specific mechanisms for the post-translational processing and modification of proteins and gene products. Appropriate cell lines or host systems can be chosen to ensure the correct modification and processing of the foreign protein expressed. To this end, eukaryotic host cells which possess the cellular machinery for proper processing of the primary transcript, glycosylation, and phosphorylation of the gene product can be used. Such mammalian host cells include but are not limited to CHO, VERO, BHK, Hela, MDCK, HEK 293, NIH 3T3, W138, BT483, Hs578T, HTB2, BT20 and T47D, NS0 (a murine myeloma cell line that does not endogenously produce any immunoglobulin chains), CRL7030, COS (e.g., COS1 or COS), PER.C6, VERO, HsS78Bst, HEK-293T, HepG2, SP210, R1.1, B-W, L-M, BSC1, BSC40, YB/20, BMT10, and HsS78Bst cells. In certain embodiments, anti-LY6K (e.g., human LY6K) antibodies described herein are produced in mammalian cells, such as CHO cells.

In a specific embodiment, the antibodies described herein have reduced fucose content or no fucose content. Such antibodies can be produced using techniques known one skilled in the art. For example, the antibodies can be expressed in cells deficient or lacking the ability to fucosylate. In a specific example, cell lines with a knockout of both alleles of α1,6-fucosyltransferase can be used to produce antibodies with reduced fucose content. The Potelligent® system (Lonza) is an example of such a system that can be used to produce antibodies with reduced fucose content.

For long-term, high-yield production of recombinant proteins, stable expression cells can be generated. For example, cell lines which stably express an anti-LY6K (e.g., human LY6K) antibody described herein can be engineered. In specific embodiments, a cell provided herein stably expresses a light chain/light chain variable region and a heavy chain/heavy chain variable region which associate to form an antigen-binding region or an antibody described herein.

In certain aspects, rather than using expression vectors which contain viral origins of replication, host cells can be transformed with DNA controlled by appropriate expression control elements (e.g., promoter, enhancer, sequences, transcription terminators, polyadenylation sites, etc.), and a selectable marker. Following the introduction of the foreign DNA/polynucleotide, engineered cells can be allowed to grow for 1-2 days in an enriched media, and then are switched to a selective media. The selectable marker in the recombinant plasmid confers resistance to the selection and allows cells to stably integrate the plasmid into their chromosomes and grow to form foci which in turn can be cloned and expanded into cell lines. This method can advantageously be used to engineer cell lines which express an anti-LY6K (e.g., human LY6K) described herein or a fragment thereof. Such engineered cell lines can be particularly useful in screening and evaluation of compositions that interact directly or indirectly with the antibody molecule.

A number of selection systems can be used, including but not limited to the herpes simplex virus thymidine kinase (Wigler M et al., (1977) Cell 11(1): 223-32), hypoxanthineguanine phosphoribosyltransferase (Szybalska E H & Szybalski W (1962) PNAS 48(12): 2026-2034), and adenine phosphoribosyltransferase (Lowy I et al., (1980) Cell 22(3): 817-23) genes in tk-, hgprt- or aprt-cells, respectively, all of which are herein incorporated by reference in their entireties. Also, antimetabolite resistance can be used as the basis of selection for the following genes: dhfr, which confers resistance to methotrexate (Wigler M et al., (1980) PNAS 77(6): 3567-70; O'Hare K et al., (1981) PNAS 78: 1527-31); gpt, which confers resistance to mycophenolic acid (Mulligan R C & Berg P (1981) PNAS 78(4): 2072-6); neo, which confers resistance to the aminoglycoside G-418 (Wu G Y & Wu C H (1991) Biotherapy 3: 87-95; Tolstoshev P (1993) Ann Rev Pharmacol Toxicol 32: 573-596; Mulligan R C (1993) Science 260: 926-932; and Morgan R A & Anderson W F (1993) Ann Rev Biochem 62: 191-217; Nabel G J & Felgner P L (1993) Trends Biotechnol 11(5): 211-5); and hygro, which confers resistance to hygromycin (Santerre R F et al., (1984) Gene 30(1-3): 147-56), all of which are herein incorporated by reference in their entireties. Methods commonly known in the art of recombinant DNA technology can be routinely applied to select the desired recombinant clone and such methods are described, for example, in Ausubel F M et al., (eds.), Current Protocols in Molecular Biology, John Wiley & Sons, NY (1993); Kriegler M, Gene Transfer and Expression, A Laboratory Manual, Stockton Press, NY (1990); and in Chapters 12 and 13, Dracopoli N C et al., (eds.), Current Protocols in Human Genetics, John Wiley & Sons, NY (1994); Colbère-Garapin F et al., (1981) J Mol Biol 150: 1-14, all of which are herein incorporated by reference in their entireties.

The expression levels of an antibody molecule can be increased by vector amplification (for a review, see Bebbington C R & Hentschel C C G, The use of vectors based on gene amplification for the expression of cloned genes in mammalian cells in DNA cloning, Vol. 3 (Academic Press, New York, 1987), which is herein incorporated by reference in its entirety). When a marker in the vector system is amplifiable, an increase in the level of inhibitor present in culture of the host cell will increase the number of copies of the marker gene. Since the amplified region is associated with the gene of interest, production of the protein will also increase (Crouse G F et al., (1983) Mol Cell Biol 3: 257-66, which is herein incorporated by reference in its entirety).

The host cell can be co-transfected with two or more expression vectors described herein, the first vector encoding a heavy chain derived polypeptide and the second vector encoding a light chain derived polypeptide. The two vectors can contain identical selectable markers which enable equal expression of heavy and light chain polypeptides. The host cells can be co-transfected with different amounts of the two or more expression vectors. For example, host cells can be transfected with any one of the following ratios of a first expression vector and a second expression vector: about 1:1, 1:2, 1:3, 1:4, 1:5, 1:6, 1:7, 1:8, 1:9, 1:10, 1:12, 1:15, 1:20, 1:25, 1:30, 1:35, 1:40, 1:45, or 1:50.

Alternatively, a single vector can be used which encodes, and is capable of expressing, both heavy and light chain polypeptides. In such situations, the light chain should be placed before the heavy chain to avoid an excess of toxic free heavy chain (Proudfoot N J (1986) Nature 322: 562-565; and Köhler G (1980) PNAS 77: 2197-2199, each of which is herein incorporated by reference in its entirety). The coding sequences for the heavy and light chains can comprise cDNA or genomic DNA. The expression vector can be monocistronic or multicistronic. A multicistronic nucleic acid construct can encode 2, 3, 4, 5, 6, 7, 8, 9, 10, or more genes/nucleotide sequences, or in the range of 2-5, 5-10, or 10-20 genes/nucleotide sequences. For example, a bicistronic nucleic acid construct can comprise, in the following order, a promoter, a first gene (e.g., heavy chain of an antibody described herein), and a second gene and (e.g., light chain of an antibody described herein). In such an expression vector, the transcription of both genes can be driven by the promoter, whereas the translation of the mRNA from the first gene can be by a cap-dependent scanning mechanism and the translation of the mRNA from the second gene can be by a cap-independent mechanism, e.g., by an IRES.

Once an antibody molecule described herein has been produced by recombinant expression, it can be purified by any method known in the art for purification of an immunoglobulin molecule, for example, by chromatography (e.g., ion exchange, affinity, particularly by affinity for the specific antigen after Protein A, and sizing column chromatography), centrifugation, differential solubility, or by any other standard technique for the purification of proteins. Further, the antibodies described herein can be fused to heterologous polypeptide sequences described herein or otherwise known in the art to facilitate purification.

In specific embodiments, an antibody described herein is isolated or purified. In certain embodiments, an isolated antibody is one that is substantially free of other antibodies with different antigenic specificities than the isolated antibody. For example, in certain embodiments, a preparation of an antibody described herein is substantially free of cellular material and/or chemical precursors. The language “substantially free of cellular material” includes preparations of an antibody in which the antibody is separated from cellular components of the cells from which it is isolated or recombinantly produced. Thus, an antibody that is substantially free of cellular material includes preparations of antibody having less than about 30%, 20%, 10%, 5%, 2%, 1%, 0.5%, or 0.1% (by dry weight) of heterologous protein (also referred to herein as a “contaminating protein”) and/or variants of an antibody, for example, different post-translational modified forms of an antibody or other different versions of an antibody (e.g., antibody fragments). When the antibody is recombinantly produced, it is also generally substantially free of culture medium, i.e., culture medium represents less than about 20%, 10%, 2%, 1%, 0.5%, or 0.1% of the volume of the protein preparation. When the antibody is produced by chemical synthesis, it is generally substantially free of chemical precursors or other chemicals, i.e., it is separated from chemical precursors or other chemicals which are involved in the synthesis of the protein. Accordingly, such preparations of the antibody have less than about 30%, 20%, 10%, or 5% (by dry weight) of chemical precursors or compounds other than the antibody of interest. In a specific embodiment, antibodies described herein are isolated or purified.

Anti-LY6K (e.g., human LY6K) antibodies or fragments thereof can be produced by any method known in the art for the synthesis of proteins or antibodies, for example, by chemical synthesis or by recombinant expression techniques. The methods described herein employ, unless otherwise indicated, conventional techniques in molecular biology, microbiology, genetic analysis, recombinant DNA, organic chemistry, biochemistry, PCR, oligonucleotide synthesis and modification, nucleic acid hybridization, and related fields within the skill of the art. These techniques are described, for example, in the references cited herein and are fully explained in the literature. See, e.g., Maniatis T et al., (1982) Molecular Cloning: A Laboratory Manual, Cold Spring Harbor Laboratory Press; Sambrook J et al., (1989), Molecular Cloning: A Laboratory Manual, Second Edition, Cold Spring Harbor Laboratory Press; Sambrook J et al., (2001) Molecular Cloning: A Laboratory Manual, Cold Spring Harbor Laboratory Press, Cold Spring Harbor, NY; Ausubel F M et al., Current Protocols in Molecular Biology, John Wiley & Sons (1987 and annual updates); Current Protocols in Immunology, John Wiley & Sons (1987 and annual updates) Gait (ed.) (1984) Oligonucleotide Synthesis: A Practical Approach, IRL Press; Eckstein (ed.) (1991) Oligonucleotides and Analogues: A Practical Approach, IRL Press; Birren B et al., (eds.) (1999) Genome Analysis: A Laboratory Manual, Cold Spring Harbor Laboratory Press, all of which are herein incorporated by reference in their entireties.

In a specific embodiment, an antibody described herein is prepared, expressed, created, or isolated by any means that involves creation, e.g., via synthesis, genetic engineering of DNA sequences. In certain embodiments, such an antibody comprises sequences (e.g., DNA sequences or amino acid sequences) that do not naturally exist within the antibody germline repertoire of an animal or mammal (e.g., human) in vivo.

In one aspect, provided herein is a method of making an anti-LY6K (e.g., human LY6K) antibody comprising culturing a cell or host cell described herein. In certain embodiments, the method is performed in vitro. In a certain aspect, provided herein is a method of making an anti-LY6K (e.g., human LY6K) antibody comprising expressing (e.g., recombinantly expressing) the antibody using a cell or host cell described herein (e.g., a cell or a host cell comprising polynucleotides encoding an antibody described herein). In certain embodiments, the cell is an isolated cell. In certain embodiments, the exogenous polynucleotides have been introduced into the cell. In certain embodiments, the method further comprises the step of purifying the antibody obtained from the cell or host cell.

In certain embodiments, an antibody is produced by expressing in a cell a polynucleotide encoding the VH and VL of an antibody described herein under suitable conditions so that the polynucleotides are expressed and the antibody is produced. In another embodiment, an antibody is produced by expressing in a cell a polynucleotide encoding the heavy chain and light chain of an antibody described herein under suitable conditions so that the polynucleotides are expressed and the antibody is produced. In certain embodiments, an antibody is produced by expressing in a cell a first polynucleotide encoding the VH of an antibody described herein, and a second polynucleotide encoding the VL of an antibody described herein, under suitable conditions so that the polynucleotides are expressed and the antibody is produced. In certain embodiments, an antibody is produced by expressing in a cell a first polynucleotide encoding the heavy chain of an antibody described herein, and a second polynucleotide encoding the light chain of an antibody described herein, under suitable conditions so that the polynucleotides are expressed and the antibody is produced.

Methods for producing polyclonal antibodies are known in the art (see, e.g., Chapter 11 in: Short Protocols in Molecular Biology, (2002) 5th Ed., Ausubel F M et al., eds., John Wiley and Sons, New York, which is herein incorporated by reference in its entirety).

Monoclonal antibodies can be prepared using a wide variety of techniques known in the art, including the use of hybridoma, recombinant, and phage display technologies, or a combination thereof. For example, monoclonal antibodies can be produced using hybridoma techniques, including those known in the art and taught, for example, in Harlow E & Lane D, Antibodies: A Laboratory Manual, (Cold Spring Harbor Laboratory Press, 2nd ed. 1988); Hammerling G J et al., in: Monoclonal Antibodies and T-Cell Hybridomas 563 681 (Elsevier, N.Y., 1981), each of which is herein incorporated by reference in its entirety. The term “monoclonal antibody” as used herein is not limited to antibodies produced through hybridoma technology. For example, monoclonal antibodies can be produced recombinantly from host cells exogenously expressing an antibody described herein or a fragment thereof, for example, light chain and/or heavy chain of such antibody.

In specific embodiments, a “monoclonal antibody,” as used herein, is an antibody produced by a single cell (e.g., hybridoma or host cell producing a recombinant antibody), wherein the antibody specifically binds to LY6K (e.g., human LY6K) as determined, e.g., by ELISA or other antigen-binding or competitive binding assay known in the art or in the examples provided herein. In certain embodiments, a monoclonal antibody can be a chimeric antibody or a humanized antibody. In certain embodiments, a monoclonal antibody is a monovalent antibody or multivalent (e.g., bivalent) antibody. In certain embodiments, a monoclonal antibody is a monospecific or multispecific antibody (e.g., bispecific antibody). Monoclonal antibodies described herein can, for example, be made by the hybridoma method as described in Kohler G & Milstein C (1975) Nature 256: 495, which is herein incorporated by reference in its entirety, or can, e.g., be isolated from phage libraries using the techniques as described herein, for example. Other methods for the preparation of clonal cell lines and of monoclonal antibodies expressed thereby are well known in the art (see, e.g., Chapter 11 in: Short Protocols in Molecular Biology, (2002) 5th Ed., Ausubel F M et al., supra).

As used herein, an antibody binds to an antigen multivalently (e.g., bivalently) when the antibody comprises at least two (e.g., two or more) monovalent binding regions, each monovalent binding region capable of binding to an epitope on the antigen. Each monovalent binding region can bind to the same or different epitopes on the antigen.

Methods for producing and screening for specific antibodies using hybridoma technology are routine and well known in the art. For example, in the hybridoma method, a mouse or other appropriate host animal, such as a sheep, goat, rabbit, rat, hamster, or macaque monkey, is immunized to elicit lymphocytes that produce or are capable of producing antibodies that will specifically bind to the protein (e.g., LY6K (e.g., human LY6K)) used for immunization. Alternatively, lymphocytes may be immunized in vitro. Lymphocytes then are fused with myeloma cells using a suitable fusing agent, such as polyethylene glycol, to form a hybridoma cell (Goding J W (Ed.), Monoclonal Antibodies: Principles and Practice, pp. 59-103 (Academic Press, 1986), herein incorporated by reference in its entirety). Additionally, a RIMMS (repetitive immunization multiple sites) technique can be used to immunize an animal (Kilpatrick K E et al., (1997) Hybridoma 16:381-9, herein incorporated by reference in its entirety).

In certain embodiments, mice (or other animals, such as rats, monkeys, donkeys, pigs, sheep, hamster, or dogs) can be immunized with an antigen (e.g., LY6K (e.g., human LY6K)), and once an immune response is detected, e.g., antibodies specific for the antigen are detected in the mouse serum, the mouse spleen is harvested and splenocytes isolated. The splenocytes are then fused by well-known techniques to any suitable myeloma cells, for example, cells from cell line SP20 available from the American Type Culture Collection (ATCC®) (Manassas, VA), to form hybridomas. Hybridomas are selected and cloned by limited dilution. In certain embodiments, lymph nodes of the immunized mice are harvested and fused with NS0 myeloma cells.

The hybridoma cells thus prepared are seeded and grown in a suitable culture medium that preferably contains one or more substances that inhibit the growth or survival of the unfused, parental myeloma cells. For example, if the parental myeloma cells lack the enzyme hypoxanthine guanine phosphoribosyl transferase (HGPRT or HPRT), the culture medium for the hybridomas typically will include hypoxanthine, aminopterin, and thymidine (HAT medium), which substances prevent the growth of HGPRT-deficient cells.

Specific embodiments employ myeloma cells that fuse efficiently, support stable high-level production of antibody by the selected antibody-producing cells, and are sensitive to a medium such as HAT medium. Among these myeloma cell lines are murine myeloma lines, such as the NS0 cell line or those derived from MOPC-21 and MPC-11 mouse tumors available from the Salk Institute Cell Distribution Center, San Diego, CA, USA, and SP-2 or X63-Ag8.653 cells available from the American Type Culture Collection, Rockville, MD, USA. Human myeloma and mouse-human heteromyeloma cell lines also have been described for the production of human monoclonal antibodies (Kozbor D (1984) J Immunol 133: 3001-5; Brodeur et al., Monoclonal Antibody Production Techniques and Applications, pp. 51-63 (Marcel Dekker, Inc., New York, 1987), each of which is herein incorporated by reference in its entirety).

Culture medium in which hybridoma cells are growing is assayed for production of monoclonal antibodies directed against LY6K (e.g., human LY6K). The binding specificity of monoclonal antibodies produced by hybridoma cells is determined by methods known in the art, for example, immunoprecipitation or by an in vitro binding assay, such as radioimmunoassay (RIA) or enzyme-linked immunoabsorbent assay (ELISA).

After hybridoma cells are identified that produce antibodies of the desired specificity, affinity, and/or activity, the clones may be subcloned by limiting dilution procedures and grown by standard methods (Goding J W (Ed.), Monoclonal Antibodies: Principles and Practice, supra). Suitable culture media for this purpose include, for example, D-MEM or RPMI 1640 medium. In addition, the hybridoma cells may be grown in vivo as ascites tumors in an animal.

The monoclonal antibodies secreted by the subclones are suitably separated from the culture medium, ascites fluid, or serum by conventional immunoglobulin purification procedures such as, for example, protein A-Sepharose, hydroxylapatite chromatography, gel electrophoresis, dialysis, or affinity chromatography.

Antibodies described herein include, e.g., antibody fragments which recognize LY6K (e.g., human LY6K), and can be generated by any technique known to those of skill in the art. For example, Fab and F(ab′)₂ fragments described herein can be produced by proteolytic cleavage of immunoglobulin molecules, using enzymes such as papain (to produce Fab fragments) or pepsin (to produce F(ab′)₂ fragments). A Fab fragment corresponds to one of the two identical arms of an antibody molecule and contains the complete light chain paired with the VH and CH1 domains of the heavy chain. A F(ab′)₂ fragment contains the two antigen-binding arms of an antibody molecule linked by disulfide bonds in the hinge region.

Further, the antibodies described herein can also be generated using various phage display methods known in the art. In phage display methods, functional antibody domains are displayed on the surface of phage particles which carry the polynucleotide sequences encoding them. In particular, DNA sequences encoding VH and VL domains are amplified from animal cDNA libraries (e.g., human or murine cDNA libraries of affected tissues). The DNA encoding the VH and VL domains are recombined together with an scFv linker by PCR and cloned into a phagemid vector. The vector is electroporated in E. coli, and the E. coli is infected with helper phage. Phage used in these methods are typically filamentous phage, including fd and M13, and the VH and VL domains are usually recombinantly fused to either the phage gene III or gene VIII. Phage expressing an antigen-binding region that binds to a particular antigen can be selected or identified with antigen, e.g., using labeled antigen or antigen bound or captured to a solid surface or bead. Examples of phage display methods that can be used to make the antibodies described herein include those disclosed in Brinkman U et al., (1995) J Immunol Methods 182: 41-50; Ames R S et al., (1995) J Immunol Methods 184: 177-186; Kettleborough C A et al., (1994) Eur J Immunol 24: 952-958; Persic L et al., (1997) Gene 187: 9-18; Burton D R & Barbas C F (1994) Advan Immunol 57: 191-280; PCT Application No. PCT/GB91/001134; International Publication Nos. WO 90/02809, WO 91/10737, WO 92/01047, WO 92/18619, WO 93/1 1236, WO 95/15982, WO 95/20401, and WO 97/13844; and U.S. Pat. Nos. 5,698,426, 5,223,409, 5,403,484, 5,580,717, 5,427,908, 5,750,753, 5,821,047, 5,571,698, 5,427,908, 5,516,637, 5,780,225, 5,658,727, 5,733,743, and 5,969,108, all of which are herein incorporated by reference in their entireties.

As described in the above references, after phage selection, the antibody coding regions from the phage can be isolated and used to generate whole antibodies, including human antibodies, or any other desired antigen-binding fragment, and expressed in any desired host, including mammalian cells, insect cells, plant cells, yeast, and bacteria, e.g., as described below. Techniques to recombinantly produce antibody fragments such as Fab, Fab′ and F(ab′)₂ fragments can also be employed using methods known in the art such as those disclosed in PCT publication No. WO 92/22324; Mullinax R L et al., (1992) BioTechniques 12(6): 864-9; Sawai H et al., (1995) Am J Reprod Immunol 34: 26-34; and Better M et al., (1988) Science 240: 1041-1043, all of which are herein incorporated by reference in their entireties.

In certain embodiments, to generate whole antibodies, PCR primers including VH or VL nucleotide sequences, a restriction site, and a flanking sequence to protect the restriction site, can be used to amplify the VH or VL sequences from a template, e.g., scFv clones. Utilizing cloning techniques known to those of skill in the art, the PCR amplified VH domains can be cloned into vectors expressing a VH constant region, and the PCR amplified VL domains can be cloned into vectors expressing a VL constant region, e.g., human kappa or lambda constant regions. The VH and VL domains can also be cloned into one vector expressing the necessary constant regions. The heavy chain conversion vectors and light chain conversion vectors are then co-transfected into cell lines to generate stable or transient cell lines that express full-length antibodies, e.g., IgG, using techniques known to those of skill in the art.

A chimeric antibody is a molecule in which different portions of the antibody are derived from different immunoglobulin molecules. For example, a chimeric antibody can contain a variable region of a mouse or rat monoclonal antibody fused to a constant region of a human antibody. Methods for producing chimeric antibodies are known in the art. See, e.g., Morrison S L (1985) Science 229: 1202-7; Oi V T & Morrison S L (1986) BioTechniques 4: 214-221; Gillies S D et al., (1989) J Immunol Methods 125: 191-202; and U.S. Pat. Nos. 5,807,715, 4,816,567, 4,816,397, and 6,331,415, all of which are herein incorporated by reference in their entireties.

A humanized antibody is capable of binding to a predetermined antigen and which comprises a framework region having substantially the amino acid sequence of a human immunoglobulin and CDRs having substantially the amino acid sequence of a non-human immunoglobulin (e.g., a murine immunoglobulin). In certain embodiments, a humanized antibody also comprises at least a portion of an immunoglobulin constant region (Fc), typically that of a human immunoglobulin. The antibody also can include the CH1, hinge, CH2, CH3, and CH4 regions of the heavy chain. A humanized antibody can be selected from any class of immunoglobulins, including IgM, IgG, IgD, IgA, and IgE, and any isotype, including IgG₁, IgG₂, IgG₃, and IgG₄. Humanized antibodies can be produced using a variety of techniques known in the art, including but not limited to, CDR-grafting (European Patent No. EP 239400; International Publication No. WO 91/09967; and U.S. Pat. Nos. 5,225,539, 5,530,101, and 5,585,089), veneering or resurfacing (European Patent Nos. EP 592106 and EP 519596; Padlan E A (1991) Mol Immunol 28(4/5): 489-498; Studnicka G M et al., (1994) Prot Engineering 7(6): 805-814; and Roguska M A et al., (1994) PNAS 91: 969-973), chain shuffling (U.S. Pat. No. 5,565,332), and techniques disclosed in, e.g., U.S. Pat. Nos. 6,407,213, 5,766,886, International Publication No. WO 93/17105; Tan P et al., (2002) J Immunol 169: 1119-25; Caldas C et al., (2000) Protein Eng. 13(5): 353-60; Morea V et al., (2000) Methods 20(3): 267-79; Baca M et al., (1997) J Biol Chem 272(16): 10678-84; Roguska M A et al., (1996) Protein Eng 9(10): 895 904; Couto J R et al., (1995) Cancer Res. 55 (23 Supp): 5973s-5977s; Couto J R et al., (1995) Cancer Res 55(8): 1717-22; Sandhu J S (1994) Gene 150(2): 409-10; and Pedersen J T et al., (1994) J Mol Biol 235(3): 959-73, all of which are herein incorporated by reference in their entireties. See also, U.S. Application Publication No. US 2005/0042664 A1 (Feb. 24, 2005), which is herein incorporated by reference in its entirety.

Methods for making multispecific antibodies (e.g., bispecific antibodies) have been described, see, e.g., U.S. Pat. Nos. 7,951,917; 7,183,076; 8,227,577; 5,837,242; 5,989,830; 5,869,620; 6,132,992; and 8,586,713, all of which are herein incorporated by reference in their entireties.

Bispecific, bivalent antibodies, and methods of making them, are described, for instance in U.S. Pat. Nos. 5,731,168; 5,807,706; 5,821,333; and U.S. Appl. Publ. Nos. 2003/020734 and 2002/0155537, each of which is herein incorporated by reference in its entirety. Bispecific tetravalent antibodies, and methods of making them are described, for instance, in Int. Appl. Publ. Nos. WO 02/096948 and WO 00/44788, the disclosures of both of which are herein incorporated by reference in its entirety. See generally, Int. Appl. Publ. Nos. WO 93/17715, WO 92/08802, WO 91/00360, and WO 92/05793; Tutt et al., J. Immunol. 147:60-69 (1991); U.S. Pat. Nos. 4,474,893; 4,714,681; 4,925,648; 5,573,920; and 5,601,819; and Kostelny et al., J. Immunol. 148:1547-1553 (1992); each of which is herein incorporated by reference in its entirety.

A bispecific antibody as described herein can be generated according to the DuoBody technology platform (Genmab A/S) as described, e.g., in International Publication Nos. WO 2011/131746, WO 2011/147986, WO 2008/119353, and WO 2013/060867, and in Labrijn A F et al., (2013) PNAS 110(13): 5145-5150. The DuoBody technology can be used to combine one half of a first monospecific antibody, or first antigen-binding region, containing two heavy and two light chains with one half of a second monospecific antibody, or second antigen-binding region, containing two heavy and two light chains. The resultant heterodimer contains one heavy chain and one light chain from the first antibody, or first antigen-binding region, paired with one heavy chain and one light chain from the second antibody, or second antigen-binding region. When both of the monospecific antibodies, or antigen-binding regions, recognize different epitopes on different antigens, the resultant heterodimer is a bispecific antibody.

The DuoBody technology requires that each of the monospecific antibodies, or antigen-binding regions, includes a heavy chain constant region with a single point mutation in the CH3 domain. The point mutations allow for a stronger interaction between the CH3 domains in the resultant bispecific antibody than between the CH3 domains in either of the monospecific antibodies, or antigen-binding regions. The single point mutation in each monospecific antibody, or antigen-binding region, is at residue 366, 368, 370, 399, 405, 407, or 409, numbered according to the EU numbering system, in the CH3 domain of the heavy chain constant region, as described, e.g., in International Publication No. WO 2011/131746. Moreover, the single point mutation is located at a different residue in one monospecific antibody, or antigen-binding region, as compared to the other monospecific antibody, or antigen-binding region. For example, one monospecific antibody, or antigen-binding region, can comprise the mutation F405L (i.e., a mutation from phenylalanine to leucine at residue 405), while the other monospecific antibody, or antigen-binding region, can comprise the mutation K409R (i.e., a mutation from lysine to arginine at residue 409), numbered according to the EU numbering system. The heavy chain constant regions of the monospecific antibodies, or antigen-binding regions, can be an IgG₁, IgG₂, IgG₃, or IgG₄ isotype (e.g., a human IgG₁ isotype), and a bispecific antibody produced by the DuoBody technology can retain Fc-mediated effector functions.

Another method for generating bispecific antibodies has been termed the “knobs-into-holes” strategy (see, e.g., Intl. Publ. WO2006/028936). The mispairing of Ig heavy chains is reduced in this technology by mutating selected amino acids forming the interface of the CH3 domains in IgG. At positions within the CH3 domain at which the two heavy chains interact directly, an amino acid with a small side chain (hole) is introduced into the sequence of one heavy chain and an amino acid with a large side chain (knob) into the counterpart interacting residue location on the other heavy chain. In certain embodiments, compositions of the disclosure have immunoglobulin chains in which the CH3 domains have been modified by mutating selected amino acids that interact at the interface between two polypeptides so as to preferentially form a bispecific antibody. The bispecific antibodies can be composed of immunoglobulin chains of the same subclass (e.g., IgG₁ or IgG₃) or different subclasses (e.g., IgG₁ and IgG₃, or IgG₃ and IgG₄).

Bispecific antibodies can, in certain instances contain, IgG₄ and IgG₁, IgG₄ and IgG₂, IgG₄ and IgG₃, or IgG₁ and IgG₃ chain heterodimers. Such heterodimeric heavy chain antibodies can routinely be engineered by, for example, modifying selected amino acids forming the interface of the CH3 domains in human IgG₄ and the IgG₁ or IgG₃, so as to favor heterodimeric heavy chain formation.

In certain embodiments, an antibody described herein, which binds to the same epitope of LY6K (e.g., human LY6K) as an anti-LY6K (e.g., human LY6K) antibody described herein, is a human antibody. In certain embodiments, an antibody described herein, which competitively blocks (e.g., in a dose-dependent manner) any one of the antibodies described herein, from binding to LY6K (e.g., human LY6K), is a human antibody. Human antibodies can be produced using any method known in the art. For example, transgenic mice which are incapable of expressing functional endogenous immunoglobulins, but which can express human immunoglobulin genes, can be used. In particular, the human heavy and light chain immunoglobulin gene complexes can be introduced randomly or by homologous recombination into mouse embryonic stem cells. Alternatively, the human variable region, constant region, and diversity region can be introduced into mouse embryonic stem cells in addition to the human heavy and light chain genes. The mouse heavy and light chain immunoglobulin genes can be rendered non-functional separately or simultaneously with the introduction of human immunoglobulin loci by homologous recombination. In particular, homozygous deletion of the JH region prevents endogenous antibody production. The modified embryonic stem cells are expanded and microinjected into blastocysts to produce chimeric mice. The chimeric mice are then bred to produce homozygous offspring which express human antibodies. The transgenic mice are immunized in the normal fashion with a selected antigen, e.g., all or a portion of an antigen (e.g., LY6K (e.g., human LY6K)). Monoclonal antibodies directed against the antigen can be obtained from the immunized, transgenic mice using conventional hybridoma technology. The human immunoglobulin transgenes harbored by the transgenic mice rearrange during B cell differentiation, and subsequently undergo class switching and somatic mutation. Thus, using such a technique, it is possible to produce therapeutically useful IgG, IgA, IgM, and IgE antibodies. For an overview of this technology for producing human antibodies, see Lonberg N & Huszar D (1995) Int Rev Immunol 13: 65-93, herein incorporated by reference in its entirety. For a detailed discussion of this technology for producing human antibodies and human monoclonal antibodies and protocols for producing such antibodies, see, e.g., International Publication Nos. WO 98/24893, WO 96/34096, and WO 96/33735; and U.S. Pat. Nos. 5,413,923, 5,625,126, 5,633,425, 5,569,825, 5,661,016, 5,545,806, 5,814,318, and 5,939,598, all of which are herein incorporated by reference in their entireties. Examples of mice capable of producing human antibodies include the XenoMouse™ (Abgenix, Inc.; U.S. Pat. Nos. 6,075,181 and 6,150,184), the HuAb-Mouse™ (Medarex, Inc./Gen Pharm; U.S. Pat. Nos. 5,545,806 and 5,569,825), the TransChromo Mouse™ (Kirin) and the KM Mouse™ (Medarex/Kirin), all of which are herein incorporated by reference in their entireties.

Human antibodies that specifically bind to LY6K (e.g., human LY6K) can be made by a variety of methods known in the art, including the phage display methods described above using antibody libraries derived from human immunoglobulin sequences. See also, U.S. Pat. Nos. 4,444,887, 4,716,111, and 5,885,793; and International Publication Nos. WO 98/46645, WO 98/50433, WO 98/24893, WO 98/16654, WO 96/34096, WO 96/33735, and WO 91/10741, all of which are herein incorporated by reference in their entireties.

In certain embodiments, human antibodies can be produced using mouse-human hybridomas. For example, human peripheral blood lymphocytes transformed with Epstein-Barr virus (EBV) can be fused with mouse myeloma cells to produce mouse-human hybridomas secreting human monoclonal antibodies, and these mouse-human hybridomas can be screened to determine ones which secrete human monoclonal antibodies that specifically bind to a target antigen (e.g., LY6K (e.g., human LY6K)). Such methods are known and are described in the art, see, e.g., Shinmoto H et al., (2004) Cytotechnology 46: 19-23; Naganawa Y et al., (2005) Human Antibodies 14: 27-31, each of which is herein incorporated by reference in its entirety.

### Kits

Also provided are kits comprising one or more antibodies described herein, or pharmaceutical compositions or conjugates thereof. In a specific embodiment, provided herein is a pharmaceutical pack or kit comprising one or more containers filled with one or more of the ingredients of the pharmaceutical compositions described herein, such as one or more antibodies provided herein. In certain embodiments, the kits contain a pharmaceutical composition described herein and any prophylactic or therapeutic agent, such as those described herein. In certain embodiments, the kits may contain a T cell mitogen, such as, e.g., phytohaemagglutinin (PHA) and/or phorbol myristate acetate (PMA), or a TCR complex stimulating antibody, such as an anti-CD3 antibody and anti-CD28 antibody. Optionally associated with such container(s) can be a notice in the form prescribed by a governmental agency regulating the manufacture, use or sale of pharmaceuticals or biological products, which notice reflects approval by the agency of manufacture, use or sale for human administration.

Also provided are kits that can be used in the above methods. In certain embodiments, a kit comprises an antibody described herein, preferably purified antibody, in one or more containers. In a specific embodiment, kits described herein contain a substantially isolated LY6K (e.g., human LY6K) antigen as a control. In another specific embodiment, the kits described herein further comprise a control antibody which does not react with LY6K (e.g., human LY6K) antigen. In another specific embodiment, kits described herein contain one or more elements for detecting the binding of an antibody to a LY6K (e.g., human LY6K) antigen (e.g., the antibody can be conjugated to a detectable substrate such as a fluorescent compound, an enzymatic substrate, a radioactive compound or a luminescent compound, or a second antibody which recognizes the first antibody can be conjugated to a detectable substrate). In specific embodiments, a kit provided herein can include a recombinantly produced or chemically synthesized LY6K (e.g., human LY6K) antigen. The LY6K (e.g., human LY6K) antigen provided in the kit can also be attached to a solid support. In a more specific embodiment, the detecting means of the above-described kit includes a solid support to which a LY6K (e.g., human LY6K) antigen is attached. Such a kit can also include a non-attached reporter-labeled anti-human antibody or anti-mouse/rat antibody. In this embodiment, binding of the antibody to the LY6K (e.g., human LY6K) antigen can be detected by binding of the said reporter-labeled antibody. In certain embodiments, the present disclosure relates to the use of a kit of the present disclosure for in vitro assaying and/or detecting LY6K (e.g., human LY6K) antigen in a biological sample.

### EXAMPLES

The following examples are offered by way of illustration and not by way of limitation.

### Example 1: Generation of Anti-LY6K Antibodies

Anti-LY6K antibodies were generated via immunization of mice in a standard RIMMS protocol using human LY6K as the immunogen at 10 ug/site. When test bleed titers of greater than 5000 by ELISA on LY6K were achieved, bone marrow and spleen were harvested. Both plasma B cells and antigen reactive B cells were sorted into single wells by FACS staining with dye labeled LY6K and standard B cell and plasma B markers and VH and VL sequences were amplified by PCR. Transient transfection of CHOs and ELISA with unique VH and VL sequences was then used to select mAbs that were reactive with LY6K by ELISA but not an irrelevant Her2-His labeled protein.

### Example 2: Selectivity of Exemplary Anti-LY6K Antibodies for Human LY6K

The selectivity of the anti-LY6K antibodies for human LY6K was measured using ELISA. Human LY6K (“hLY6K”), cynomolgus monkey LY6K (“cyLY6K”), rat LY6K (“rLY6K”), and human LY6E (“hLY6E”) sourced from R&D Systems were coated on High Bind ELISA plates in PBS overnight. The plates were washed and blocked in PBS 1% BSA and probed with the anti-LY6K antibodies formatted as human IgG1 monoclonal antibodies at 10 μg/ml in PBS 1% BSA. After 1 hour, the plates were washed and probed with an anti-human F(ab)2 HRPO conjugate (for hLY6K, cyLY6K, and rLY6K) or an anti-human Ig Fe gamma HRPO conjugate (for hLY6E) for 1 hour, followed by washing, addition of TMB substrate, and plate reading. Binding data for hLY6K, cyLY6K, rLY6K, and hLY6E are shown in Table 7 (expressed as a percentage of the maximum value for a commercial anti-LY6K mAb on human LY6K).

TABLE 7
Binding of anti-LY6K antibodies to indicated protein.

| Ab | hLY6K | cyLY6K | rLY6K | LY6E |
|---|---|---|---|---|
| 2659 | ND | ND | ND | ND |
| 2660 | 95.1 | 106.2 | −0.1 | 3.1 |
| 3035 | 65.2 | 71.4 | 1 | 5.6 |
| 3047 | 6.2 | 3 | 1.5 | 1.7 |
| 3048 | 81.2 | 0.1 | 1.5 | 2.5 |
| 3055 | 22.6 | 40.3 | 0.4 | 26 |
| 3056 | ND | ND | ND | ND |
| 3063 | 71.6 | 2.6 | −0.2 | 4.2 |
| 3074 | 89.2 | −0.2 | −1.2 | 3.4 |
| 3075 | 58.3 | 16.7 | 0.7 | 1.6 |
| 3082 | 87.2 | 95.2 | −0.5 | 2.1 |
| 3090 | ND | ND | ND | ND |
| 3096 | 35 | 37.2 | 0 | 1 |
| 3101 | 94.9 | 3 | 0.6 | 2.4 |
| 3102 | 38.4 | 40.1 | 0.2 | 3.8 |
| 3481 | 99.4 | 0.2 | 0.9 | 2.7 |
| 3501 | 85.6 | 2 | 0.4 | 4.3 |
| 3503 | 66.6 | 76.4 | 0.7 | 2.9 |
| 3507 | 2.6 | 2.6 | −1.2 | 1.7 |
| 3514 | 4 | 2.6 | 0 | 2.9 |
| 3518 | 67 | 2.7 | 2.1 | 2.1 |
| 3520 | 61.2 | 13.6 | 0.7 | 2.8 |
| 3522 | 2.1 | 2.6 | 0.7 | 4.3 |
| 3527 | 54.6 | 58 | 1.1 | 4.6 |
| 3529 | 3.6 | 1.5 | 0.4 | 2.8 |
| 3553 | 26 | 28.9 | 2.4 | 5 |
| 3704 | 2.2 | 2 | 0.6 | 3.2 |
| 3705 | 102.5 | 1.1 | 0 | 7.8 |
| 3719 | 48.8 | 0.8 | −1.7 | 4.2 |
| 3724 | 6.2 | 3.2 | 3.3 | 3.9 |
| 3728 | 96.4 | 110.4 | −1.3 | 1 |
| 3741 | 67.6 | 66.4 | −1.6 | 3.1 |
| 3743 | 105.6 | 3.5 | 2.6 | 5 |
| 3746 | 76.9 | 78.7 | 3.1 | 1.2 |
| 3785 | 43.7 | 57.2 | 2.5 | 4.5 |
| 3815 | 20.2 | 29.1 | 2.2 | 3.5 |

ND: not determined

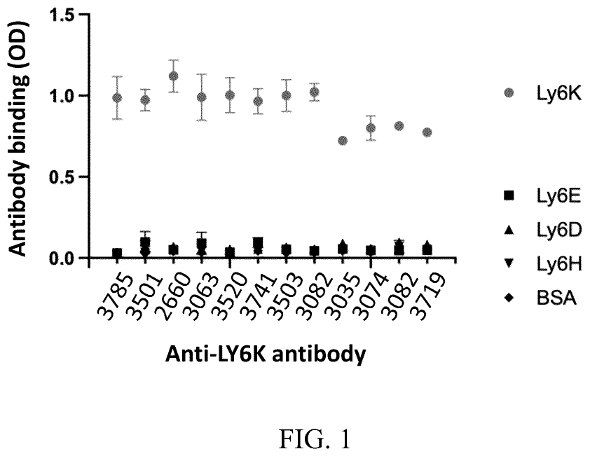

The selectivity of a subset of anti-LY6K antibodies for LY6K over various LY6 family members was measured using ELISA. LY6K, LY6E, LY6D, and LY6H sourced from R&D Systems, along with BSA as a negative control, were coated on High Bind ELISA plates in PBS overnight. The plates were washed and blocked in PBS 1% BSA and probed with the subset of anti-LY6K antibodies formatted as human IgG1 monoclonal antibodies at 10 μg/ml in PBS 1% BSA. After 1 hour, the plates were washed and probed with an anti-human F(ab)2 HRPO conjugate for 1 hour, followed by washing, addition of TMB substrate, and plate reading. As shown in FIG. 1, each of the anti-LY6K antibodies selectively binds LY6K.

### Example 3: Affinity of Exemplary Anti-LY6K Antibodies for Human LY6K

The apparent affinity of a subset of the anti-LY6K antibodies for cell-surface anchored LY6K was measured using flow cytometry where primary anti-LY6K mAbs were incubated in 96-well U bottom plates with 10(5) target cells for 30 minutes at 4 degrees C., washed twice by spinning at 1500 RPM and 2nd stage goat F(ab)2 anti-human Fc gamma dye labeled polyclonal (Southern goat 1012-09) added at 1:200 dilution for 30 minutes. Cells were then washed and analyzed on a FACSCAN for Mean Fluorescence, and these values were used in Prism to calculate the apparent affinity for each of the subset of anti-LY6K antibodies for cell-surface anchored LY6K, represented as half maximal effective concentration (EC50), as shown in Table 8. The apparent affinity of an anti-LY6K antibody commercially available from R&D Systems (#MAB6648, referred to as “R&D” in Table 8) was also determined as a comparison.

TABLE 8
Apparent affinity (EC50) of exemplary anti-LY6K
antibodies to cell-surface anchored LY6K.

| Ab | EC50 (nM) |
|---|---|
| 2660 | 0.3645 |
| 3035 | 1.04039E+13 |
| 3048 | 3.334 |
| 3063 | 90.53 |
| 3074 | 163.8 |
| 3075 | 0.8055 |
| 3082 | 3.901E+30 |
| 3481 | 2.839 |
| 3501 | 13098 |
| 3503 | 0.576 |
| 3520 | 3.61 |
| 3705 | 58.08 |
| 3741 | 1.97E+20 |
| 3743 | 1.181 |
| 3746 | 3.206 |
| R&D | 0.7235 |

The apparent affinity of a subset of the anti-LY6K antibodies for LY6K was also measured using an ELISA assay in which individual antibodies were HRP-labeled using an Abcam Kit (ab102890). High Bind ELISA plates were coated with 2 μg/mL of recombinant human LY6K (Wuxi) overnight, washed and blocked in PBS 1% BSA. HRPO linked anti-LY6K mAbs were then added as a titration for 1 hour, after which the plate was washed, and TMB was added before reading on a plate GloMax plate reader at 450 nM. The apparent affinity for each of the subset of anti-LY6K antibodies for LY6K, generated using Graphpad Prism and represented as half maximal effective concentration (EC50), is shown in Table 9. The apparent affinity of an anti-LY6K antibody commercially available from R&D Systems (“R&D”) was also determined as a comparison.

TABLE 9
Apparent affinity (EC50) of exemplary
anti-LY6K antibodies to LY6K.

| Ab | EC50 (nM) |
|---|---|
| 2660 | 1.57 |
| 3035 | 2.862 |
| 3048 | 2.65 |
| 3063 | 4.674 |
| 3074 | 5.196 |
| 3075 | 1.557 |
| 3082 | 4.593 |
| 3481 | 2.08 |
| 3501 | 18.59 |
| 3503 | 2.675 |
| 3520 | 2.344 |
| 3705 | 16.2 |
| 3741 | 3.078 |
| 3743 | 2.317 |
| 3746 | 3.035 |
| R&D | 2.504 |

The affinity of each of a subset of the anti-LY6K antibodies for LY6K (“hLY6K”), cynomolgus monkey LY6K (“cyLY6K”), and rat LY6K (“rLY6K”) was measured using surface plasmon resonance. To measure binding kinetics, a capture and binding assay was performed on the Carterra LSA system. Briefly, an amine-coupled anti-human IgG lawn was generated using an HC30M chip with anti-human IgG-Fc specific antibody (Jackson Immuno-Research). All test antibodies were expressed at 1 mL scale in CHO cells for 6 days, and supernatant was cleared of cells by centrifugation. CHO supernatants were diluted to approximately 1 μg/mL antibody and injected over the capture chip, resulting in a captured antibody array. Non-regenerative kinetics were measured by injection of hLY6K-his, hLY6K-mFc, cyLY6K-mFc, or rLY6K-mFc starting at a concentration of 1.37N-9 M, with 3-fold increase in concentration up to 1.266-6 M. Each analyte injection was flowed over the array for a 5-minute association phase, followed by a 10-minute buffer dissociation phase. Binding was detected and relative response units (RU) were collected for each antibody on the surface. KD values were determined using the Carterra software. The results are shown in Table 10.

TABLE 10
Affinity of exemplary anti-LY6K antibodies
for hLY6K, cyLY6K, and rLY6K.

| | hLY6K-his | hLY6K-mFc | cyLY6K-mFc | rLY6K-mFc |
|---|---|---|---|---|
| Ab | KD (M) | KD (M) | KD (M) | KD (M) |
| 2659 | 6.1329E−09 | 3.4039E−11 | N/A | N/A |
| 2660 | 3.5941E−09 | 7.017E−12 | 5.1847E−11 | N/A |
| 3035 | weak | N/A | N/A | N/A |
| 3047 | N/A | 2.7184E−06 | 2.1628E−05 | 1.2945E−07 |
| 3048 | 4.6953E−09 | 4.278E−11 | N/A | N/A |
| 3056 | 4.7249E−10 | 2.3252E−11 | N/A | N/A |
| 3074 | 3.2337E−09 | 1.6394E−10 | N/A | N/A |
| 3082 | 2.0628E−09 | 4.4412E−11 | 1.0264E−10 | 2.3992E−07 |
| 3096 | 6.3751E−08 | 1.3586E−09 | 9.4857E−09 | N/A |
| 3101 | 1.8261E−09 | 2.6441E−11 | N/A | N/A |
| 3481 | 1.1897E−09 | 1.5692E−11 | N/A | N/A |
| 3501 | N/A | 7.205E−10 | N/A | N/A |
| 3503 | 3.4906E−09 | 5.5255E−11 | 1.9694E−10 | N/A |
| 3518 | 2.747E−08 | 2.5442E−10 | N/A | N/A |
| 3520 | 3.191E−08 | 3.7316E−10 | 1.1709E−07 | N/A |
| 3522 | N/A | N/A | N/A | weak |
| 3705 | 6.3113E−09 | 1.8533E−10 | N/A | N/A |
| 3719 | N/A | 4.3025E−10 | N/A | N/A |
| 3728 | 4.96E−09 | 7.335E−12 | 7.8634E−11 | N/A |
| 3741 | 5.6589E−09 | 5.048E−11 | 3.7902E−10 | N/A |
| 3743 | 7.0032E−10 | 4.5046E−11 | N/A | N/A |
| 3746 | 2.5014E−09 | 3.615E−11 | 2.1571E−10 | N/A |
| 3785 | 7.4845E−09 | 2.6254E−10 | 1.3911E−09 | N/A |
| 3815 | 1.0957E−07 | 7.6286E−10 | 1.1734E−08 | N/A |

N/A: low/no binding,
KD not measurable

### Example 4: Sensitivity of Exemplary Anti-LY6K Antibodies for Detection of LY6K

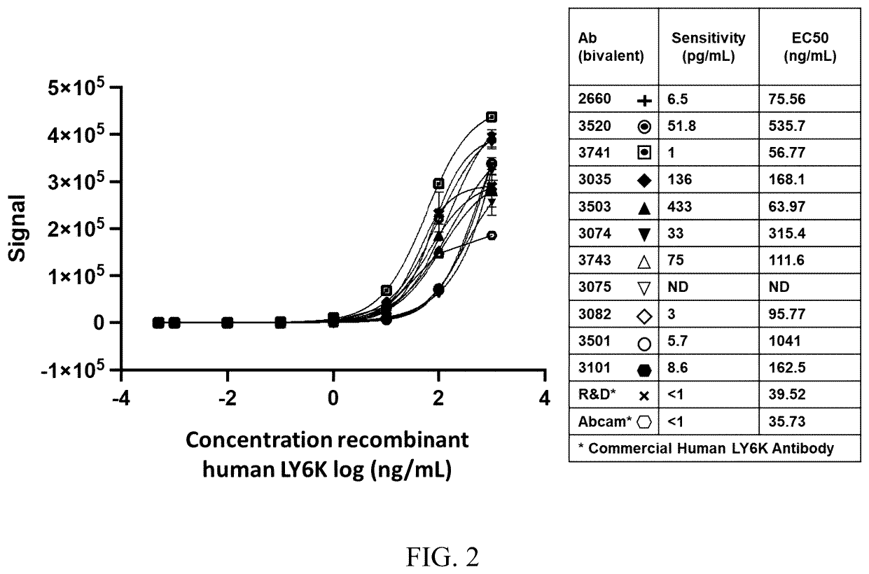

Capture ELISA was used to determine the sensitivity of a subset of the anti-LY6K antibodies for detecting LY6K. Two commercially available anti-LY6K antibodies were used as a reference (R&D Systems MAB6648 and Abcam ab246486). The capture antibodies were diluted to 2 μg/mL and added to the plate (MSD L55XB-3). Human LY6K (2 ug/mL Wuxi LY\hat{ }KGLY138(1-38TR)_8His) was used. A commercial polyclonal anti-hLY6K antibody (R&D Systems, AF6648) was sulfo-tagged (R31AA-1) according to manufacturer protocol and used at 200 ng/mL for detection. The results are shown in FIG. 2. The EC50 for LY6K was determined by Prism software by nonlinear regression. Sensitivity refers to lower limit of detection, which was determined using MSD Discover workbench software and calculated as the concentration 2.5 standard deviations above the lowest point on the calibration (no antigen).

### Example 5. Internalization of Anti-LY6K Antibodies

Temperature sensitive internalization of exemplary LY6K antibodies was tested in OVCAR-8 cells. The anti-LY6K antibodies were used at 1 ug/mL to stain 2000,000 OVCAR-8 cells for 6 hr at either 4 degrees or 37 degrees Celsius. The IgG isotypes were used as a negative control (Jackson 015-000-003, 009-000-003) and trastuzumab (Absolute antibodies T1722A01) was used as a positive control. Cells were washed two times, and a fluorescent secondary was incubated on ice for 40 min (Jackson 109-116-190 1:200 dilution, Southern Biotech 1012-09 1:100 dilution). Cells were washed two times and fixed with fixation buffer (BD 554655). Cells were washed, and fluorescence was recorded using Attune Nxt (Invitrogen A24858). The percent internalization was calculated based on the geometric mean fluorescence fold increase of each antibody, with 4 degrees Celsius set to max fluorescence.

pH sensitive internalization of exemplary LY6K antibodies was tested in OVCAR-8 and HEKLY6K expressing cells. The cells were seeded into a tissue culture white plate at 30,000 cells/well (50 uL) and incubated overnight in phenol red free media supplemented with FBS (Gibco 11835-030). The Fabfluor-pH sensitive antibody (Satorius 4722) was incubated with individual anti-LY6K antibodies or control antibodies trastuzumab (Absolute antibodies T1722A01) and huIgG1 (Wuxi at a 1:3 molar ratio and incubated for 20 mins). The Fabfluor antibody mix (50 uL) was added to the cell plate at a final concentration of 2 μg/mL (13.33 nM) and incubated for 18 hr on OVCAR-8 cells or 2 hr on HEKLY6K cells. After incubation, cells were washed two times with PBS in the plate. Cells were removed from the plate by versene (ThermoFischer Scientific 15040066) and washed two times. Fluorescence was recorded on an Attune Nxt (Invitrogen A24858). The precent internalization on OVCAR-8 cells was calculated based on the geometric mean fluorescence of each antibody with trastuzumab set to max fluorescence. The percent internalization on HEKLY6K cells was calculated based on each antibody's geometric mean fluorescence with the highest mean fluorescence intensity (MFI) for the group, 3035, set to max fluorescence (100% internalization).

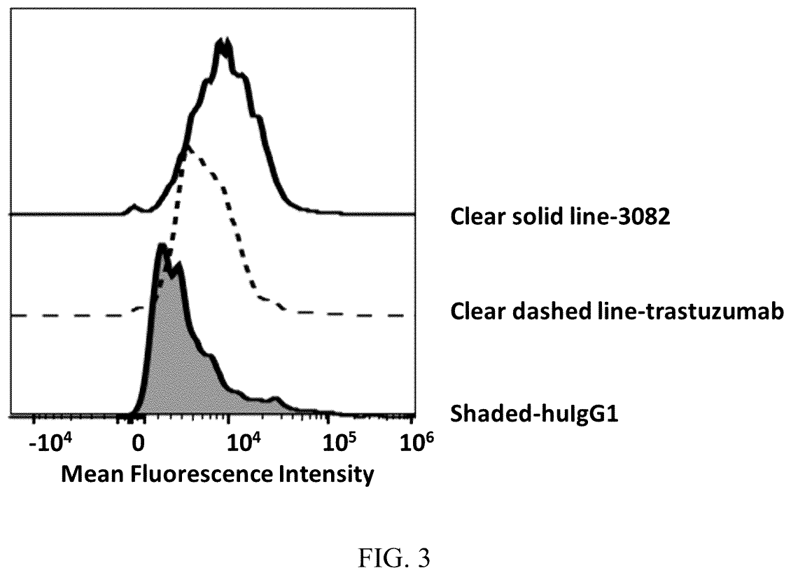

The temperature sensitive internalization and pH sensitive internalization results for the exemplary anti-LY6K antibodies are shown in Table 11, and a representative fluorescence plot for antibody 3082 labeled with pH-sensitive dye is shown in FIG. 3. The figure demonstrates that anti-LY6K antibody 3082 (clear solid line) internalized and endocytosed, releasing fluorescent signal, more than negative control huIgG1 (shaded black) by an increase in MFI due to a decrease in pH in OVCAR-8 cells. Trastuzumab, a known commercially available internalizer (dashed line), was used as a positive control.

TABLE 11
Percent internalization of exemplary anti-LY6K antibodies.

| | Temperature | Fab-Fluor pH | Fab-Fluor pH |
|---|---|---|---|
| | sensitive | sensitive | sensitive |
| | internalization | internalization | internalization |
| Ab | (OVCAR-8) | (OVCAR-8) | (HEKLY6K) |
| 3082 | 47.5 | 247.44 | 96.8 |
| 3035 | 19.2 | 220.4 | 100 |
| 3481 | 26.2 | 218.86 | 56.2 |
| 3705 | 30.7 | 218.52 | 68.9 |
| 3074 | 48.6 | 127.73 | 45.6 |
| 3075 | 32.2 | 125.51 | 38.6 |
| 2660 | 60.5 | 118 | 98.8 |
| 3503 | 26.4 | 108 | 66 |
| 3048 | 50.7 | 85.34 | 55.6 |
| 3520 | 37 | 82.27 | 30.4 |
| 3743 | 48.3 | 63.52 | 72.3 |
| 3047 | ND | 19.15 | ND |
| 3101 | ND | 15.8 | ND |
| 3741 | 65.4 | −5.06 | 27.6 |
| 3063 | 92.6 | −10.97 | ND |
| 3527 | ND | −26.19 | ND |
| 3785 | ND | −32.33 | 14.8 |
| 3096 | ND | −51.99 | ND |
| 3518 | ND | −53.52 | ND |
| 3815 | ND | −59.89 | ND |
| 3501 | 93.8 | −62.5 | 2.1 |
| 3102 | ND | −67.61 | ND |

### Example 6. Epitope Binning of Exemplary Anti-LY6K Antibodies

Epitope binning for a subset of the anti-LY6K antibodies was performed using a competitive immunoassay. The exemplary anti-LY6K antibodies, along with a commercial anti-LY6K antibody (R&D MAB6648; “R&D” in Table 12), were HRP-labeled using a Lightning link HRP conjugation Kit (Abcam ab10289). The HRP conjugated antibodies were titrated and EC80 values were determined. Each individual purified anti-LY6K antibody had a corresponding HRP-conjugated antibody, which was used against itself to calculate percent competition. If the antigen of the non-HRP capture mAb prevented 60% of the binding of the competing HRP-labeled antibody to human LY6K (2 ug/mL Wuxi LY\hat{ }KGLY138(1-38TR)_8His), then these mAbs were considered to bind similar or overlapping epitopes. Conversely, if the binding of the unlabeled antibody to the LY6K did not interfere with the binding of the other HRP-labeled antibody, then they were considered to bind to distinct, non-overlapping epitopes. The binning results are shown in Table 12.

TABLE 12
Epitope binning of exemplary anti-LY6K antibodies.

| 1 | 2 | 3 | Not Determined |
|---|---|---|---|
| 3075 | 2660/3728 | 3082 | 3501 |
| 3520 | 3035 | 3748/3503 | 3785 |
| | 3048 | 3741 | |
| | 3074 | | |
| | 3481 | | |
| | 3705 | | |
| | 3743 | | |
| | 3048 | | |
| | 3063 | | |
| | R&D | | |

### Example 7. Detection of LY6K Via Immunohistochemistry Using Exemplary Anti-LY6K Antibodies

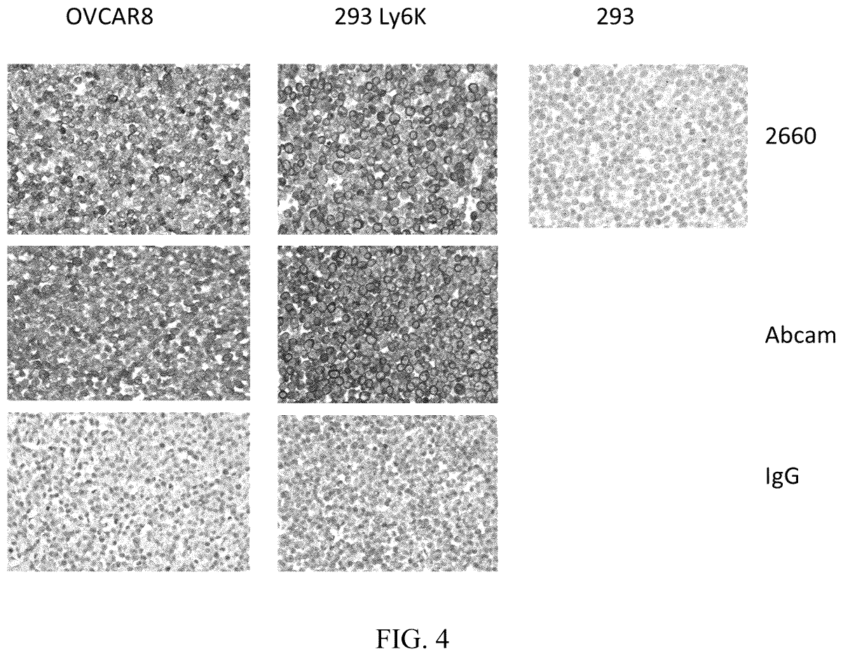

Exemplary anti-LY6K antibodies were used to detect LY6K in 293 cells overexpressing LY6K, OVCAR-8 cells, and 293 cells (negative control) via immunohistochemistry (IHC). Briefly, sections were processed by removing paraffin and rehydrating, incubated 20 mins at 4 degrees Celsius, washed, put in Bloxall for 10 minutes, followed by normal serum for 30 minutes, and stained overnight with an anti-LY6K antibody formatted as a human IgG1 monoclonal antibody or with Abcam (ab246486) anti-LY6K peptide (positive control) at 5 μg/ml in PBS 5% mouse serum followed by biotinylated anti-rabbit or anti-human IgG for 30 minutes, AB 30 minutes, and then hemtaxoylin stained. Exemplary IHC images for antibody 2660 are shown in FIG. 4, along with positive control (Abcam anti-LY6K) and negative control (IgG) images. IHC results for the remaining anti-LY6K antibodies are summarized in Table 13.

TABLE 13
IHC results for exemplary anti-LY6K antibodies.

| Ab | IHC 293 LY6K | IHC OVCAR8 | IHC 293 |
|---|---|---|---|
| 2659 | ND | ND | ND |
| 2660 | ++ | ++ | − |
| 3035 | + | + | − |
| 3047 | − | NA | − |
| 3048 | + | +/− | − |
| 3055 | +/− | +/− | − |
| 3056 | ND | ND | ND |
| 3063 | + | − | − |
| 3074 | ++ | +/− | − |
| 3075 | − | NA | − |
| 3082 | − | +/− | − |
| 3090 | ND | ND | ND |
| 3096 | +/− | − | − |
| 3101 | ++ | ++ | − |
| 3102 | ++ | +/− | − |
| 3481 | + | + | − |
| 3501 | + | + | − |
| 3503 | − | +/− | − |
| 3507 | − | NA | − |
| 3514 | + | − | − |
| 3518 | − | NA | − |
| 3520 | − | NA | − |
| 3522 | − | NA | − |
| 3527 | − | NA | − |
| 3529 | − | NA | − |
| 3553 | ++ | +/− | − |
| 3704 | − | NA | − |
| 3705 | + | ++ | − |
| 3719 | − | NA | − |
| 3724 | − | − | − |
| 3728 | ++ | NA | − |
| 3741 | − | NA | − |
| 3743 | − | NA | − |
| 3746 | − | NA | − |
| 3785 | − | NA | − |
| 3815 | ND | ND | ND |

(++) Best;
(+) Positive;
(+/−) Marginal or diffuse stain;
(−) No stain or nonspecific;
(NA) Did not stain OVCAR-8 if 293 LY6K result was negative;
(ND) not determined

### Example 8. Tumor Cell Killing with Anti-LY6K Antibody-Based T Cell Engagers

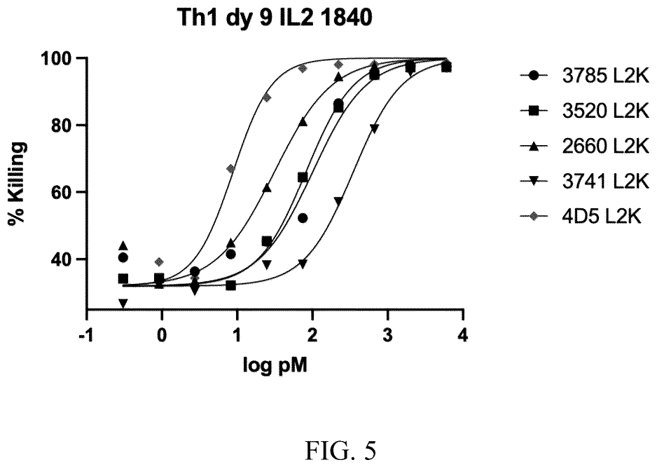

Exemplary anti-LY6K antibodies were used to make L2K formatted bispecific T cell engagers targeting CD3. Th1 lymphocytes were generated by activation for 4 days with Miltenyi Transact beads in media (RPMI 10% FBS+NEAA and P/S), followed by 9 days in culture with media+5 ng/ml IL2, were placed in RPMI 5% FBS+NEAA+P/S at 10(6) cells/ml with OVCAR-8 GFP luciferase lines at 10(5) cell/ml. Anti-LY6K T cell engagers were added at various concentrations using a 4D5-based 1+1 T cell engager against Her2 as a comparison. After 18 hours, luciferin was added to 200 μg/ml and luminescence readings were taken. The results are shown in FIG. 5.

### Example 9. Epitope Mapping of Exemplary Anti-LY6K Antibodies

Epitopes were mapped for five exemplary anti-LY6K antibodies (2660, 3481, 3501, 3520, and 3741) using linear and conformational epitope mapping.

Peptide synthesis: to reconstruct epitopes of LY6K, a library of linear peptide-based mimics was synthesized using Fmoc-based solid-phase peptide synthesis. An amino functionalized polypropylene support was obtained by grafting with a proprietary hydrophilic polymer formulation, followed by reaction with t-butyloxycarbonyl-hexamethylenediamine (BocHMDA) using dicyclohexylcarbodiimide (DCC) with N-hydroxybenzotriazole (HOBt) and subsequent cleavage of the Boc-groups using trifluoroacetic acid (TFA). Standard Fmoc-peptide synthesis was used to synthesize peptides on the amino-functionalized solid support by custom modified JANUS liquid handling stations (Perkin Elmer). Three sets of linear peptides were synthesized. The first set included peptides of length 7 derived from the sequence of human LY6K (SEQ ID NO: 232) with an offset of one residue. The second set included peptides of length 10 derived from the sequence of human LY6K (SEQ ID NO: 232) with an offset of one residue. The third set included peptides of length 15 derived from the sequence of human LY6K (SEQ ID NO: 232) with an offset of one residue.

Conformational epitope structural mimics were synthesized using the Chemically Linked Peptides on Scaffolds (CLIPS™) technology (Biosynth). See, e.g., Timmerman et al., 2007, J Mol. Recognit. 20:283-299 and Langedijk et al., 2011, Analytical Biochemistry 417:149-155. CLIPS technology allows peptides to adopt thermodynamically favored conformations, including single loops, double-loops, triple loops, sheet-like folds, helix-like folds, and combinations thereof. CLIPS scaffolds are coupled to cysteine residues. The side-chains of multiple cysteines in the peptides are coupled to a CLIPS scaffold with two or three reactive groups. For example, a 0.5 mM solution of the P2 CLIPS (2,6-bis(bromomethyl)pyridine) was dissolved in ammonium bicarbonate (20 mM, pH 7.8)/acetonitrile (1:3 (v/v)), and this solution was added onto the peptide arrays where the CLIPS template bound to side-chains of two cysteines introduced in the solid-phase bound peptides of the peptide-arrays. The peptide arrays were gently shaken in the solution for 30 to 60 minutes while completely covered in solution. Finally, the peptide arrays were washed extensively with excess of H₂O and sonicated in disruption buffer (1% SDS/0.1% 2,2′-(Ethylenedioxy)diethanethiol in PBS (pH 7.2)) at 70° C. for 30 minutes, followed by sonication in H₂O for another 45 minutes. The T3 CLIPS carrying peptides were made in a similar way with three cysteines. Three sets of conformational structural mimic peptides were synthesized. The first set included constrained single loop peptides of length 15, where positions 2-14 were 13-mer peptides derived from the sequence of human LY6K (SEQ ID NO: 232) with an offset of one residue. Cys residues were inserted on positions 1 and 15 and joined by mP2 CLIPS in order to create a loop mimic. Native Cys residues were replaced by Cys-acm. The second set included β-turn peptide mimics of length 22, where positions 2-21 were 20-mer peptides derived from the sequence of human LY6K (SEQ ID NO: 232) with an offset of one residue. Residues on positions 11 and 12 were replaced by a “PG” motif in order to induce the β-turn formation. Cys residues were inserted on positions 1 and 22 and joined by mP2 CLIPS in order to stabilize the mimic. Native Cys residues were replaced by Cys-acm. The third set included α-helical peptide mimics of length 19 derived from the sequence of human LY6K (SEQ ID NO: 232) with an offset of one residue. Cys residues were inserted on positions 1 and 5 and joined by means of mP2 CLIPS to nucleate an α-helical structure. Native Cys residues were replaced by Cys-acm (denoted “2”).

To verify the quality of the synthesized peptides, a separate set of positive and negative control peptides was synthesized in parallel. These were screened with commercial antibodies 3C9 and 57.9 (see Posthumus et al., 1990, J. Virol. 64:3304-3309).

ELISA screening: antibody binding to each of the synthesized peptides was tested via ELISA. Peptide arrays were incubated with primary antibody solution overnight at 4° C. After washing, the peptide arrays were incubated with a 1/1000 dilution of a goat anti-human HRP conjugate antibody (Southern Biotech, cat. #2010-05) for one hour at 25° C. After washing, the peroxidase substrate 2,2′-azino-di-3-ethylbenzthiazoline sulfonate (ABTS) and 20 μl/ml of 3 percent H₂O₂ were added. After one hour, the color development was measured. The color development was quantified with a charge coupled device (CCD) camera and an image processing system. The values obtained from the CCD camera ranged from 0 to 3000 mAU, similar to a standard 96-well plate ELISA-reader. Occasionally a well contains an air-bubble, resulting in a false-positive value. Such false-positives are scored as 0 by manual inspection.

The sample screens were optimized for sample concentration and blocking conditions for each sample until clear signals over background values were observed. Generally, peaks are defined as at least two times the background value (defined as the value for the isotype control screening for each peptide for the specific peptide mimic). In the absence of an isotype control, the individual antibody samples were used as internal controls, and distinctly different binding regions were observed. A binding event was only noted if multiple overlapping peaks were present within this binding region. Putative core epitopes were identified from adjacent peptides with similar or maximally 20% lower intensity than the top peak within a binding region. In addition, if peaks were well-defined, containing multiple overlapping peptides, lower intensity peaks were also included. For full evaluation of putative epitopes, information was taken from multiple mimic types where possible.

All five antibodies showed distinct binding peaks in different regions of the canonical human LY6K sequence. The putative core epitopes are listed in Table 14. The residue numbers refer to the position of the putative core epitope sequence in the canonical human LY6K sequence set forth in SEQ ID NO: 232. Underlined sequences represent the most likely core epitope sequence.

TABLE 14
Putative core epitopes of
exemplary anti-LY6K antibodies.

| | | Putative core | SEQ |
|---|---|---|---|
| Ab | Residues | epitope sequence | ID NO |
| 2660 | 16-30 | WTDANLTARQRDPED | 233 |
| 3481 | 25-34 | QRDPEDSQRT | 235 |
| 3501 | 18-29 | DANLTARQRDPE | 236 |
| 3520 | 52-61 | FECQNPRRCK | 237 |
| 3741 | 97-106 | PEEKRFLLEE | 238 |

As demonstrated in Examples 2 and 3, some of the exemplary anti-LY6K antibodies, including 2660, 3520, and 3741, are cross-reactive and bind both human LY6K and cynomolgus monkey LY6K (“cyLY6K”). Furthermore, antibodies 2660, 3520, and 3741 seem to bind different epitopes within LY6K, based on the putative epitopes shown in Table 14 and on the binning data described in Example 6. The amino acid sequence of cyLY6K was compared (i.e., via BLAST alignment) to that of human LY6K to evaluate whether the exemplary cross-reactive anti-LY6K antibodies may be binding similar epitopes. As shown in Table 15, the cyLY6K amino acid sequences corresponding to the putative epitope regions in Table 14 have a high degree of similarity to the human LY6K epitope sequences for the cross-reactive anti-LY6K antibodies. As in Table 14, underlined sequences in Table 15 represent the most likely core epitope sequence. The regions from rat LY6K (“rLY6K”) and mouse LY6K (“mLY6K”) corresponding to the putative epitope for the 2660 anti-LY6K antibody are also shown in Table 15. As demonstrated in Examples 2 and 3, the 2660 antibody did not react with rLY6K.

TABLE 15
Putative cross-reactive epitopes
of exemplary anti-LY6K antibodies.

| | | Putative core | SEQ |
|---|---|---|---|
| Ab | Species | epitope sequence | ID NO |
| 2660* | Human LY6K | WTDANLTARQRDPED | 233 |
| | (residues 16-30) | | |
| | cyLY6K | WTHLNLTARQQDPED | 239 |
| | rLY6K | KTRHPLEMVILLALL | 241 |
| | mLY6K | QSNALTCHVCEAQNS | 243 |
| 3481 | Human LY6K | QRDPEDSQRT | 235 |
| | (residues 25-34) | | |
| | cyLY6K | QQDPEDTPQT | 245 |
| 3501 | Human LY6K | DANLTARQRDPE | 236 |
| | (residues 18-29) | | |
| | cyLY6K | HLNLTARQQDPE | 246 |
| 3520* | Human LY6K | FECQNPRRCK | 237 |
| | (residues 52-61) | | |
| | cyLY6K | FECENPRRCQ | 247 |
| 3741* | Human LY6K | PEEKRFLLEE | 238 |
| | (residues 97-106) | | |
| | cyLY6K | PEEKRFLLEE | 238 |

*antibody displayed cross-reactivity with cyLY6K (see Examples 2 and 3)

The invention is not to be limited in scope by the specific embodiments described herein. Indeed, various modifications of the invention in addition to those described will become apparent to those skilled in the art from the foregoing description and accompanying figures. Such modifications are intended to fall within the scope of the appended claims.

All references (e.g., publications or patents or patent applications) cited herein are incorporated herein by reference in their entireties and for all purposes to the same extent as if each individual reference (e.g., publication or patent or patent application) was specifically and individually indicated to be incorporated by reference in its entirety for all purposes.

Other embodiments are within the following claims.

What is claimed:

1. An antibody that specifically binds human LY6K, the antibody comprising: a VH comprising the CDRH1, CDRH2, and CDRH3 amino acid sequences of the VH amino acid sequence set forth in any one of SEQ ID NOs: 1-34; and a VL comprising the CDRL1, CDRL2, and CDRL3 amino acid sequences of the VL amino acid sequence set forth in any one of SEQ ID NOs: 35-68.

2. An antibody that specifically binds human LY6K, the antibody comprising:
(a) a VH comprising the CDRH1, CDRH2, and CDRH3 amino acid sequences of the VH amino acid sequence set forth in SEQ ID NO: 16; and a VL comprising the CDRL1, CDRL2, and CDRL3 amino acid sequences of the VL amino acid sequence set forth in SEQ ID NO: 50;
(b) a VH comprising the CDRH1, CDRH2, and CDRH3 amino acid sequences of the VH amino acid sequence set forth in SEQ ID NO: 2; and a VL comprising the CDRL1, CDRL2, and CDRL3 amino acid sequences of the VL amino acid sequence set forth in SEQ ID NO: 36; or
(c) a VH comprising the CDRH1, CDRH2, and CDRH3 amino acid sequences of the VH amino acid sequence set forth in SEQ ID NO: 17; and a VL comprising the CDRL1, CDRL2, and CDRL3 amino acid sequences of the VL amino acid sequence set forth in SEQ ID NO: 51.

3. The antibody of claim 2, wherein the antibody comprises:
(a) the CDRH1, CDRH2, and CDRH3 amino acid sequences, respectively, set forth in SEQ ID NOs: 74, 105, and 138;
(b) the CDRH1, CDRH2, and CDRH3 amino acid sequences, respectively, set forth in SEQ ID NOs: 70, 91, and 125; or
(c) the CDRH1, CDRH2, and CDRH3 amino acid sequences, respectively, set forth in SEQ ID NOs: 74, 106, and 139.

4. The antibody of claim 2, wherein the antibody comprises:
(a) the CDRL1, CDRL2, and CDRL3 amino acid sequences, respectively, set forth in SEQ ID NOs: 170, 185, and 211;
(b) the CDRL1 amino acid sequence set forth in SEQ ID NO: 159, the CDRL2 amino acid sequence WAS, and the CDRL3 amino acid sequence set forth in SEQ ID NO: 199; or
(c) the CDRL1, CDRL2, and CDRL3 amino acid sequences, respectively, set forth in SEQ ID NOs: 171, 187, and 212.

5. The antibody of claim 2, wherein the antibody comprises the CDRH1, CDRH2, CDRH3, CDRL1, CDRL2, and CDRL3 amino acid sequences, respectively, set forth in SEQ ID NOs: 74, 105, 138, 170, 185, and 211.

6. The antibody of claim 2, wherein the antibody comprises the CDRH1, CDRH2, CDRH3, and CDRL1 amino acid sequences, respectively, set forth in SEQ ID NOs: 70, 91, 125, and 159, the CDRL2 amino acid sequence WAS, and the CDRL3 amino acid sequence set forth in SEQ ID NO: 199.

7. The antibody of claim 2, wherein the antibody comprises the CDRH1, CDRH2, CDRH3, CDRL1, CDRL2, and CDRL3 amino acid sequences, respectively, set forth in SEQ ID NOs: 74, 106, 139, 171, 187, and 212.

8. The antibody of claim 2, wherein the antibody comprises the VH amino acid sequence of SEQ ID NO: 16, 2, or 17.

9. The antibody of claim 2, wherein the antibody comprises the VL amino acid sequence of SEQ ID NO: 50, 36, or 51.

10. The antibody of claim 2, wherein the VH and VL comprise the amino acid sequences, respectively, set forth in SEQ ID NOs: 16 and 50.

11. The antibody of claim 2, wherein the VH and VL comprise the amino acid sequences, respectively, set forth in SEQ ID NOs: 2 and 36.

12. The antibody of claim 2, wherein the VH and VL comprise the amino acid sequences, respectively, set forth in SEQ ID NOs: 17 and 51.

13. The antibody of claim 2, wherein the antibody comprises a heavy chain constant region, or an Fc region thereof.

14. The antibody of claim 13, wherein the heavy chain constant region is selected from the group consisting of a human IgG1, IgG2, IgG3, IgG4, IgA1, and IgA2.

15. The antibody of claim 13, wherein the heavy chain constant region is a human IgG1.

16. The antibody of claim 13, wherein the heavy chain constant region is a variant of a wild-type heavy chain constant region, wherein the variant heavy chain constant region binds to an Fc gamma receptor (FcγR) with lower affinity than the wild-type heavy chain constant region binds to the FcγR.

17. The antibody of claim 16, wherein the amino acid sequence of the heavy chain constant region comprises a mutation selected from the group consisting of L234A, L235A, and a combination thereof, numbered according to the EU numbering system.

18. The antibody of claim 16, wherein the amino acid sequence of the heavy chain constant region comprises a mutation selected from the group consisting of H₄₃₅R, Y436F, and a combination thereof, numbered according to the EU numbering system.

19. The antibody of claim 16, wherein the amino acid sequence of the heavy chain constant region comprises a mutation selected from the group consisting of L234A, L235A, H₄₃₅R, Y436F, and a combination of two or more thereof, numbered according to the EU numbering system.

20. The antibody of claim 2, wherein the antibody comprises a heavy chain constant region comprising the amino acid sequence of SEQ ID NO: 228 or 229.

21. The antibody of claim 2, wherein the antibody comprises a light chain constant region.

22. The antibody of claim 21, wherein the light chain constant region is a human kappa or lambda constant region.

23. The antibody of claim 2, wherein the antibody is conjugated to a cytotoxic agent, cytostatic agent, toxin, radionuclide, or detectable label.

24. The antibody of claim 2, wherein the antibody is a multispecific antibody comprising a CD3-binding region, optionally wherein the CD3-binding region comprises a single-chain fragment variable (scFv) that specifically binds to CD3, optionally human CD3.

25. A polynucleotide or plurality of polynucleotides encoding a VH and/or a VL, or a heavy chain and/or a light chain of the antibody of claim 1.

26. A vector or plurality of vectors comprising the polynucleotide or plurality of polynucleotides of claim 25.

27. A recombinant host cell comprising the polynucleotide or plurality of polynucleotides of claim 25.

28. A composition comprising the antibody of claim 1, or a polynucleotide or plurality of polynucleotides encoding the antibody, and a pharmaceutically acceptable carrier or excipient.

29. A method of producing an antibody, the method comprising culturing the recombinant host cell of claim 27 under suitable conditions such that the polynucleotide or plurality of polynucleotides is expressed, and the antibody is produced.

30. A method of treating cancer in a subject, the method comprising administering to the subject an effective amount of the antibody of claim 1.


SEQUENCE LISTING (247 sequences)

SEQ ID NO: 1 - protein, 117 aa, Synthetic construct
EVQLVESGGGLVQPGRSLRLSCAASGFTFDDYAMHWVRQAPGKGLEWVSGISWKSGIIGYADSVKGRFTISRDNAKNSLYLQMNSLRAEDTALYYCAKDSRTGLDYWGQGTLVTVSS

SEQ ID NO: 2 - protein, 117 aa, Synthetic construct
EVQLVESGGGLVKPGGSLRLSCAASGFTFSDVWMSWVRQAPGKGLEWVGRIKSKTDGGRIDYAAPVKGRFTISRDDSKNTLYLQMNSLKTEDTAVYYCTTVPYSDYWGQGTLVTVSS

SEQ ID NO: 3 - protein, 116 aa, Synthetic construct
EVQLVESGGGLVQPGGSLKLSCAASGFTFSDSTLHWVRQASGKGLEWVGRIRSKANSYATAYAASVKGRFTISRDDSKNTAYLQMNSLKTEDTAVYYCHNWSYGYWGQGTLVTVSS

SEQ ID NO: 4 - protein, 115 aa, Synthetic construct
EVQLVESGGGLVQPGGSLRLSCAASGFTFSSYSMNWVRQAPGKGLEWVSGINWNGGSTGYADSVKGRFTISRDNAKNSLYLQMNSLRAEDTALYYCARGRLFDYWGQGTLVTVSS

SEQ ID NO: 5 - protein, 117 aa, Synthetic construct
EVQLVESGGGLVQPGRSLRLSCAASGFTFDDYAMHWVRQAPGKGLEWVSGITWKSGTIGYADSVKGRFTISRDNAKNSLYLQMNSLRAEDTALYYCAKDSRTGLDYWGQGALVTVSS

SEQ ID NO: 6 - protein, 118 aa, Synthetic construct
EVQLVESGGGLVQPGRSLRLSCAASGFTFDDFAMHWVRQAPGKGLEWVSGISWNTGTIGYADSVKGRFTISRDNAKNSLYLQMNSLRAEDTALYYCAKDKGTTGFDYWGQGTLVTVSS

SEQ ID NO: 7 - protein, 117 aa, Synthetic construct
EVQLVESGGGLVQPGRSLRLSCAASGFTYDDYAMHWVRQVPGKGLEWVSGITWKKSIIGYADSVRGRFTVSRDSAKNSLYLQMSSLRVEDTALCYCAKDSTDGLDYWGQGTLVTVSS

SEQ ID NO: 8 - protein, 117 aa, Synthetic construct
EVQLVESGGGLVKPGRSLRLSCAASGFTFDDFAMHWVRQAPGKGLEWVSGLSWKGHTLGYADSVKGRFTFSRDSAKNSLYLQMNSLRAEDTALYYCAKDTRSGLDYWGQGTLVTVSS

SEQ ID NO: 9 - protein, 118 aa, Synthetic construct
EVQLVESGGGLVHPGRSLRLSCAASGFTFDDYAMHWVRQAPGKGLEWVSGISWKTNKIDYADSVKGRFTTSRDNAKNSLYLQMNSLRTEDTAFYYCAKDRREMTFDIWGQGTMVTVSS

SEQ ID NO: 10 - protein, 117 aa, Synthetic construct
QVQLVQSGSELKRPGASVKVSCKASGYTFIRYAMNWVRQAPGQGLEWMGWINTNTGNPTYAQGFTGRFVLSVDTSVSTAYLQISSLKVEDTAVYYCGRPGISSFEYWGQGTLVTVSS

SEQ ID NO: 11 - protein, 128 aa, Synthetic construct
EVQLVESGGGLVKPGGSLRLSCAASGFTFSSSSMNWVRQAPGKGLEWVSSISRSSYYIYYADSVEGRFTISRDNAKNSLYLQMNSLRAEDTAVYYCAKIGSSSEGVEDYYYYYYMDVWGKGTTVTVSS

SEQ ID NO: 12 - protein, 121 aa, Synthetic construct
QVQLQESGPGLVKPSGTLSLTCAVSGGSISRSNWWSWVRQTPGKGLDWIGKIYHSGSTNYNPSLKSRVTISVDKSKNRFSLNLSSVTAADTAVYYCASSGSHYYYNYMEVWGKGTTVTVSS

SEQ ID NO: 13 - protein, 114 aa, Synthetic construct
QVQLVQSGAEVKKPGASVRVSCKASGYTFTGYYIHWVRQAPGQGLEWMGWINPNSGGTNYAQKFQDRVTMTRDTSISTVYMELSRLRSDDTAVFFCAREASNFWGQGTLVTVSS

SEQ ID NO: 14 - protein, 117 aa, Synthetic construct
EVQLVESGGGLVQPGRSLRLSCAASGFTFDDYAMHWVRQTPGKGLEWVSGITWKSGNIGYADSVKGRFTISRDNAKNSLYLQMNSLRAEDTALYYCAKDTEAGLDYWGQGTLVTVSS

SEQ ID NO: 15 - protein, 114 aa, Synthetic construct
QVQLVQSGAEVKKPGASVKVSCKASGYTFTGYYIHWMRQAPGQGLEWMGWINPNSGGTNYAQNFQGRVTMTRDTSINTAYMELSRLKSDDTAVYYCAKESGSPWGQGTLVTVSS

SEQ ID NO: 16 - protein, 117 aa, Synthetic construct
EVQLVESGGGLVQSGRSLRLSCAASGFTFDDYAMHWVRQAPGKGLEWVSGITWKDVTIGYADSVKGRFTISRDNAKNSLYLQMNSLGAEDTALYYCAKDTSDGLDYWGQGTLVTVSS

SEQ ID NO: 17 - protein, 121 aa, Synthetic construct
EVQLVESGGGLVQPGRSLRLSCAASGFTFDDYAMHWVRQAPGKGLEWVSGISWNSGTIDYADSVKGRFTISRDNAKNSLYLQVNSLRAEDTALYYCAKDSPQKDYYYMDVWGKGTTVTVSS

SEQ ID NO: 18 - protein, 125 aa, Synthetic construct
EVQLVESGGGLVKPGGSLRLSCAASGFIFSSYSMNWVRQAPGKGLEWVSSISSSSSYIYYADSEKGRFTISRDNAKNSLYLQMNSLRAEDTAVYYCARQSTVVTSDYYYYYMDVWGKGTTVTVSS

SEQ ID NO: 19 - protein, 121 aa, Synthetic construct
EVQLVESGGGLVKPGGSLRLSCAASGFTFSSYSMNWVRQAPGKGLEWVSSISSSSDYIYYADSVKGRFTISRDDAKNSLYLQMNSLRAEDTAVYYCARGLEPNYYYYMDVWGKGTTVTVSS

SEQ ID NO: 20 - protein, 121 aa, Synthetic construct
QVQLVQSGAEVKKPGASVKVSCKASGYTFTSYGISWVRQATGQGLEWMGWMNPNSGNTGYAQKFQGRVTMTRNTSISTAYMELSSLRSEDTAVYYCRAAGYYYYYYYMDVWGKGTTVTVSS

SEQ ID NO: 21 - protein, 116 aa, Synthetic construct
EVQLVESGGGLVQPGGSLKLSCAASGFTFSGSAMNWVRQASGKGLEWVGRIRSKANSYATAYAASLKGRFTISRDDSKNTAYLQMNSLKTEDTAVYYCTVTPFDYWGQGTLVTVSS

SEQ ID NO: 22 - protein, 114 aa, Synthetic construct
QVQLVQSGSELKKPGASVKVSCKASGYTFTLYAMNWVRQAPGQGLEWMGWINTNTGNPTYAQGFTGRFVFSLDTSVSTAYLQISSLKAEDTAVYYCARTNWNSWGQGTMVTVSS

SEQ ID NO: 23 - protein, 123 aa, Synthetic construct
QVQLQQSGPGLVKPSQTLSLTCAISGDSVSSNSAAWNWIRQSPSRGLEWLGRTYYRSKWYNDYAVSVKSRITINPDTSKNQFSLQLNSVTPEDTAVYYCARECSGSYYRFDPWGQGTLVTVSS

SEQ ID NO: 24 - protein, 128 aa, Synthetic construct
EVQLVESGGGLVKPGGSLRLSCAASGFTFSSYSMNWVRQAPGKGLEWVSSISSTSSYIYYADSVKGRFTISRDNAKNSLYLQMNSLRAEDTAVYYCARSDYYDSGSYKNYYYYYMDVWGKGTTVTVSS

SEQ ID NO: 25 - protein, 126 aa, Synthetic construct
EVQLVESGGGVVRPGGSLRLSCAASGFTFDDYGMSWVRQAPGKGLEWVSGINWNGGSTGYADSVKGRFTISRDNAKNSLYLQMNSLRAEDTALYYCARENILTGYYNLYYYYMDVWGKGTTVTVSS

SEQ ID NO: 26 - protein, 114 aa, Synthetic construct
QVQLVQSGAEVKKPGASVKVSCKASGYTFTGYYMHWVRQAPGTGLDWMGWISPNSGGTNYAQKFQGRVTMTRDTSISTVYMELSRLRSDDTALYYCAREVGNYWGQGTLVTVSS

SEQ ID NO: 27 - protein, 117 aa, Synthetic construct
EVQLVESGGGLVQPGRSLRLSCAASGFTFDDYAMHWVRQAPGKGLEWVSGISWKIITIGYADSVKGRFTISRDNVKNSLYLQMNSLRAEDTALYYCAKDSRTGLDYWGQGTLVTVSS

SEQ ID NO: 28 - protein, 118 aa, Synthetic construct
EVQLVESGGGLVHPGRSLRLSCAASGFTFDDYAMHWVRQAPGKGLEWVSGISWKNGKIDYADSVKGRFITSRDNAKNSLYLQMNSLRTEDTALYYCAKDRIEMTFDIWGQGTMVTVSS

SEQ ID NO: 29 - protein, 121 aa, Synthetic construct
EVQLVESGGGLVQPGRSLRLSCTASGFTFNDYAMHWVRQAPGKGLEWVSGISWNSNNIDYADSVKGRFTISRDNAKNSLYMQMNSLRAEDTALYYCAKDGPGTTNDAFDIWGQGTMVTVSS

SEQ ID NO: 30 - protein, 124 aa, Synthetic construct
QLQLQESGPGLVKPSETLSLTCTVSGGSINSKSYYWDWIRQPPGKGLEWIGNIYYSGSTYYNPSLKSRVTISVDTSKNQFSLKLSSVTATDTAVYYCARHRGVTVTTYWYFDLWGRGTLVTVSS

SEQ ID NO: 31 - protein, 124 aa, Synthetic construct
EVQLVESGGGLVKPGGSLRLSCAASGFTFSSYSMNWVRQAPGKGLEWVSSISSSSNYIYYVDSVKGRFTTSRDNAKNSLYLQMNSLRAEDTAVYYCARQEATGNNYYYYYMDVWGKGTTVTVSS

SEQ ID NO: 32 - protein, 118 aa, Synthetic construct
EVQLVESGGGLVHPGRSLRLSCAASGFTFDDYAMHWVRQAPGKGLEWVSGISWKSGRIDYADSVKGRFTISRDNAKNSLYLQMNSLRAEDTALYYCAKDRRELTFDIWGQGTMVTVSS

SEQ ID NO: 33 - protein, 127 aa, Synthetic construct
QVQLQQSGPGLVKPSQTLSLTCAISGDSVSSNSAAWNWIRQSPSRGLEWLGRTYFRSKWNNDYTLSVKNRITINPDTSKNHFSLQLNSVTPEDTAVYYCAGGGYCNRTYCPDVFDIWGQGTMVTVSS

SEQ ID NO: 34 - protein, 117 aa, Synthetic construct
EVQLVESGGGVVRPGGSLRLSCAASGFTFDDYGMSWVRQAPGKGLEWVSGINWNGGSTGYVDSVKGRFTISRDNAKNSLYLQMNSLRAEDTALYYCARMFYGAFDIWGQGTMVTVSS

SEQ ID NO: 35 - protein, 112 aa, Synthetic construct
DIVMTQTPLSSPVTLGQPASISCRSSQSLVHSDGNTYLSWLQQRPGQPPRLLIYKISNRFSGVPDRFSGSGAGTDFTLKISRVEAEDVGVYYCMQATQFPYTFGQGTKLEIK

SEQ ID NO: 36 - protein, 113 aa, Synthetic construct
DIVMTQSPDSLAVSLGERATINCKSSQSVLYSSDNENYLAWYQQKPGQPPKLLIYWASSRESGVPDRFSGSGSGTDFTLTISSLQAEDVAVYYCHQYYNTPWTFGQGTKVEIK

SEQ ID NO: 37 - protein, 109 aa, Synthetic construct
QTVVTQEPSLTVSPGGTVTLTCASSTGAVTSYYYPNWFQQKPGQAPRTVIYSTTNKHSWTPARFSGSLLGGKAALTLSGVLPEDEADYYCLLFYGGAYVFGTGTKVTVL

SEQ ID NO: 38 - protein, 114 aa, Synthetic construct
DIVMTQSPDSLAVSLGERATINCKSSQSVLYSSNNKNYLAWYQQKPGQPPKLLIYWASTRESGVPDRFSGSGSGTDFTLTISSLQAEDVAVYYCQQYYSTPSYTFGQGTKLEIK

SEQ ID NO: 39 - protein, 112 aa, Synthetic construct
DIVMTQSPLSSPVTLGQPASISCRSSQSLVHSDGNTYLSWLQQRPGQPPRLLIYKISNRFSGVPDRFSGSGAGTDFTLKISRVEAEDVGVYYCMQATQFPYTFGQGTRLEIK

SEQ ID NO: 40 - protein, 107 aa, Synthetic construct
DIQMTQSPSSVSASVGDRVTITCRASQGISRWLVWYQQKPGKAPKLLIYAASSLQSGVPSRFSGSGSGTDFTLTISSLQPEDFATYYCQQANSFPRTFGQGTRLEIK

SEQ ID NO: 41 - protein, 112 aa, Synthetic construct
DIVMTQTPLSSPVTLGQPASLSCRSSQSLLHSNGNTYLSWLQQRPGQPPRLLIYKISNRFSGVPDRFSGSGAGTDFTLKISRVEGEDVGVYYCMQATEFPYTFGQGTKLEIK

SEQ ID NO: 42 - protein, 112 aa, Synthetic construct
DIVMTQTPLSSPVTLGQPASISCRSSQSLEHSDGNTYLSWLQQRPGQPPRLLIYKISNRFSGVPDRFSGNGAGTDFTLKISRVEAEDVGIYYCMQATEFPFTFGGGTKVEIK

SEQ ID NO: 43 - protein, 112 aa, Synthetic construct
DIVMTQTPLSSPVTLGQPASISCRSSQSLEHSNGNTYLSWLQQRPGQPPRLLIYKISNRFSGVPDRFSGSGAGTDFTLKISRVEAEDVGIYYCMQATQFPLTFGGGTKVEIK

SEQ ID NO: 44 - protein, 107 aa, Synthetic construct
DIQMTQSPSSLSASVGDRVAITCRATQDISNYLDWYQQKPGKAPKLLIYDASNLETGVPSRFSGSGSGTDFTFTINSLQPEDIATYYCQQYDNLPLTFGGGTKVEIK

SEQ ID NO: 45 - protein, 107 aa, Synthetic construct
DIQMTQSPSSLSASVGDRVTITCRASQNINSYLNWYQQKPGKAPKLLIYAASSLQSGVPSRFSGSGSGTDFTLTISSLQPEDFATYYCHQSYSTPLTFGGGTKVEIK

SEQ ID NO: 46 - protein, 108 aa, Synthetic construct
EIVLTQSPGTLSLSPGERATLSCRASQTVSSSYLVWYQQKPGQAPRLLIYGASGRATGIPDRFSGSGSGTDFTLTISRLEPEDFAVFYCQHYGSSPYTFGQGTKLEIK

SEQ ID NO: 47 - protein, 107 aa, Synthetic construct
DIQMTQSPSSLSASIGDRVTITCQASQDISNYLNWYQQKPGKAPKLLIYDASNLETGVPSRFSGSGSGTDFTFTISSLQPEDIATYYCQQYESLPFNFGGGTKVEIK

SEQ ID NO: 48 - protein, 112 aa, Synthetic construct
DIVMTQTPLSSPVTLGQPASISCRSSQSLVHSNGNTYLSWLQQRPGQPPRLLIYKISNRFSGVPDRFSGSGAGTDFTLKISRVEAEDVGVYYCMQATQFPYTFGQGTKLEIK

SEQ ID NO: 49 - protein, 107 aa, Synthetic construct
DIQMTQSPSSLSASVGDRVTITCQASQDISNYLNWYQQKPGKAPKLLIYDASNLERGVPSRFSGSGSGTDFTFSISSLQPEDIATYHCQEYDSLPFTFGPGTKVDIK

SEQ ID NO: 50 - protein, 112 aa, Synthetic construct
DIVMTQTPLSSPVTLGQPASISCRSSQSLVHSNGNTYLSWLQQRPGQPPRLLIYKISNRFSGVPDRFSGSGAGTDFTLKISRVETEDVGIYYCMQAIQFPYTFGQGTKLEIK

SEQ ID NO: 51 - protein, 107 aa, Synthetic construct
DIQMTQSPSSLSASVGDRVTITCRTSQSISSYLNWYQQKPGKAPNLLIYAASSLQSGVPSRFSGSGSGTDFTLTISSLQPEDFATYYCQQSYITPITFGQGTRLEIK

SEQ ID NO: 52 - protein, 107 aa, Synthetic construct
DIQMTQSPSSLSASVGDRVTITCRASQSISNYLNWYQQKPGKAPNLLIYAASSLQSGVPSRFSGSGSGTDFTLTISSLQPEDFATYYCQQSYSPPITFGQGTRLEIK

SEQ ID NO: 53 - protein, 108 aa, Synthetic construct
EIVMTQSPGTLSVSPGERATLSCRASQSVSSSYLAWYQQKPGQAPRLLIYGASTRATGIPARFSGSGSGTEFTLTISSLQSEDFAVYYCQQYNNWPLTFGGGTKVEIK

SEQ ID NO: 54 - protein, 111 aa, Synthetic construct
DIVMTQSPLSLPVTPGEPASISCRSSQSLLHSNGYNYLDWYLQKPGQSPQLLIYLGSNRASGVPDRFSGSGSGTDFTLKISRVEAEDVGVYYCMQALQTYTFGQGTKLEIK

SEQ ID NO: 55 - protein, 107 aa, Synthetic construct
DIQMTQSPSSLSASVGDRVTITCQASQDISNHLNWYQQKPGKAPKLLINDASNLETGVPSRFSGSGSGTDFTFTISSLQPEDIATYYCQQYDNVPYTFGQGTKLEIK

SEQ ID NO: 56 - protein, 107 aa, Synthetic construct
DIQMTQSPSSLSASVGDRVTITCQASQDISNYLHWFQQKPGKAPKLLIYDASNLETGVPSRFSGSGSGTDFTFTIFSLQPEDIATYYCQHYDNLPLTFGGGTKVEIK

SEQ ID NO: 57 - protein, 112 aa, Synthetic construct
DIVMTQTPLSSPVTLGQPASISCRSSQSLVHSNGNTYLSWLQQRPGQPPRLLIYKISHRFSGVPDRFSGSGAGTDFTLKIRRVEAEDVGVYYCMQATQFPYTFGQGTKLEIK

SEQ ID NO: 58 - protein, 107 aa, Synthetic construct
DIQMTQSPSSLSASVGDRVTITCRTSQSISSYLNWFQQKPGKAPKVLIYAASSLQRGVPSRFSGSGSGTDFTLTISSLQPEDFATYYCQQSYSTPLTFGGGTKVEIK

SEQ ID NO: 59 - protein, 105 aa, Synthetic construct
DIQMTQSPSSLSASVGDRVTITCRASQSISSYLNWYQQKPGKAPKLLIYAASSLQSGVPSRFSGSGSGTDFTLTISSLQPEDFATYYCQQSYSTSHGGGTKVEIK

SEQ ID NO: 60 - protein, 107 aa, Synthetic construct
DIQMTQSPTSLSTSVGDRVTITCQASQDIYNYLNWYQLKPGKAPKLLIYDASNLERGVTSRFSGSGSGTDFTFTISSLQPEDIATYYCQQYDSLPITFGQGTRLEIK

SEQ ID NO: 61 - protein, 107 aa, Synthetic construct
DIQMTQSPSSLSASVGDRVTITCRASQTISSYLNWYQQKPGKAPKLLIYAASSLQSGVPSRFSGSGSGTDFTLTISSLQPEDFATYYCQQTYTAPLTFGPGTKVDIK

SEQ ID NO: 62 - protein, 112 aa, Synthetic construct
DIVMTQSPLSSPVTLGQPASISCRSSQSLEHSNGNTYLSWLQQRPGQPPRLLIYKISNRFSGVPDRFSGSGAGTDFTLKISRVEAEDVGVYYCMQTTQFPLTFGGGTKVEIK

SEQ ID NO: 63 - protein, 107 aa, Synthetic construct
DIQMTQSPSSLSASVGDRVTITCRASHSISSYLNWYQQKPGKAPKLLIYAASSLQSGVPSRFSGSGSGTDFTLTISSLQPEDFATYYCHQGYSTPLTFGGGTKVEIK

SEQ ID NO: 64 - protein, 107 aa, Synthetic construct
EIVLTQSPDFQSVTPKEKVTITCRASQSIGNSLHWYQQKSDQSPKLLIKYVSQSFSGVPSRFSGSGSGTDFTLTINSLEAEDAATYYCHQSSDFPHTFGQGTKLEIK

SEQ ID NO: 65 - protein, 107 aa, Synthetic construct
DIQMTQSPSSLSASVGDRVTITCRASQSISNYLNWYQQNLGKAPKVLIYAASSLQSGVPSRFSGSGSGTDFTLTISSLQPEDFATYYCQQTYSTPITFGQGTRLEIK

SEQ ID NO: 66 - protein, 112 aa, Synthetic construct
DIVMTQTPLSSPVTLGQPASISCRSSQSLEHSNGNTYLSWLQQRPGQPPRLLIYKISNRFSGVPDRFSGSGAGTDFTLKISRVEAEDVGVYYCMQTTQFPLTFGGGTKVEIK

SEQ ID NO: 67 - protein, 107 aa, Synthetic construct
DIQMTQSPSSLSASVGDRVTFTCRSSQSISSYLNWYQQKPGKAPNLLIYVASTLQSGVPSRFSGSGSGTDFTLTISSLQPEDFATYYCQQSYSIPLTFGGGTKVEIK

SEQ ID NO: 68 - protein, 108 aa, Synthetic construct
EIVLTQSPGTLSLSPGERATLSCRASQSVSSNYLAWYQQKPGQAPRLLIYGASSRATGIPDRFSGSGSGTDFTLTISRLEPEDFAVYYCQQYGSSPFTFGQGTKVDIK

SEQ ID NO: 69 - protein, 8 aa, Synthetic construct
GFTFDDYA

SEQ ID NO: 70 - protein, 8 aa, Synthetic construct
GFTFSDVW

SEQ ID NO: 71 - protein, 8 aa, Synthetic construct
GFTFSDST

SEQ ID NO: 72 - protein, 8 aa, Synthetic construct
GFTFSSYS

SEQ ID NO: 73 - protein, 8 aa, Synthetic construct
GFTFDDFA

SEQ ID NO: 74 - protein, 5 aa, Synthetic construct
DYAMH

SEQ ID NO: 75 - protein, 5 aa, Synthetic construct
DFAMH

SEQ ID NO: 76 - protein, 5 aa, Synthetic construct
RYAMN

SEQ ID NO: 77 - protein, 5 aa, Synthetic construct
SSSMN

SEQ ID NO: 78 - protein, 6 aa, Synthetic construct
RSNWWS

SEQ ID NO: 79 - protein, 5 aa, Synthetic construct
GYYIH

SEQ ID NO: 80 - protein, 5 aa, Synthetic construct
GYYIH

SEQ ID NO: 81 - protein, 5 aa, Synthetic construct
SYSMN

SEQ ID NO: 82 - protein, 5 aa, Synthetic construct
SYGIS

SEQ ID NO: 83 - protein, 5 aa, Synthetic construct
GSAMN

SEQ ID NO: 84 - protein, 5 aa, Synthetic construct
LYAMN

SEQ ID NO: 85 - protein, 7 aa, Synthetic construct
SNSAAWN

SEQ ID NO: 86 - protein, 5 aa, Synthetic construct
DYGMS

SEQ ID NO: 87 - protein, 5 aa, Synthetic construct
GYYMH

SEQ ID NO: 88 - protein, 7 aa, Synthetic construct
SKSYYWD

SEQ ID NO: 89 - protein, 5 aa, Synthetic construct
DVWMS

SEQ ID NO: 90 - protein, 8 aa, Synthetic construct
ISWKSGII

SEQ ID NO: 91 - protein, 10 aa, Synthetic construct
IKSKTDGGRI

SEQ ID NO: 92 - protein, 10 aa, Synthetic construct
IRSKANSYAT

SEQ ID NO: 93 - protein, 8 aa, Synthetic construct
INWNGGST

SEQ ID NO: 94 - protein, 8 aa, Synthetic construct
ITWKSGTI

SEQ ID NO: 95 - protein, 8 aa, Synthetic construct
ISWNTGTI

SEQ ID NO: 96 - protein, 17 aa, Synthetic construct
GITWKKSIIGYADSVRG

SEQ ID NO: 97 - protein, 17 aa, Synthetic construct
GLSWKGHTLGYADSVKG

SEQ ID NO: 98 - protein, 17 aa, Synthetic construct
GISWKTNKIDYADSVKG

SEQ ID NO: 99 - protein, 17 aa, Synthetic construct
WINTNTGNPTYAQGFTG

SEQ ID NO: 100 - protein, 17 aa, Synthetic construct
SISRSSYYIYYADSVEG

SEQ ID NO: 101 - protein, 16 aa, Synthetic construct
KIYHSGSTNYNPSLKS

SEQ ID NO: 102 - protein, 17 aa, Synthetic construct
WINPNSGGTNYAQKFQD

SEQ ID NO: 103 - protein, 17 aa, Synthetic construct
GITWKSGNIGYADSVKG

SEQ ID NO: 104 - protein, 17 aa, Synthetic construct
WINPNSGGTNYAQNFQG

SEQ ID NO: 105 - protein, 17 aa, Synthetic construct
GITWKDVTIGYADSVKG

SEQ ID NO: 106 - protein, 17 aa, Synthetic construct
GISWNSGTIDYADSVKG

SEQ ID NO: 107 - protein, 17 aa, Synthetic construct
SISSSSSYIYYADSEKG

SEQ ID NO: 108 - protein, 17 aa, Synthetic construct
SISSSSDYIYYADSVKG

SEQ ID NO: 109 - protein, 17 aa, Synthetic construct
WMNPNSGNTGYAQKFQG

SEQ ID NO: 110 - protein, 19 aa, Synthetic construct
RIRSKANSYATAYAASLKG

SEQ ID NO: 111 - protein, 18 aa, Synthetic construct
RTYYRSKWYNDYAVSVKS

SEQ ID NO: 112 - protein, 17 aa, Synthetic construct
SISSTSSYIYYADSVKG

SEQ ID NO: 113 - protein, 17 aa, Synthetic construct
GINWNGGSTGYADSVKG

SEQ ID NO: 114 - protein, 17 aa, Synthetic construct
WISPNSGGTNYAQKFQG

SEQ ID NO: 115 - protein, 17 aa, Synthetic construct
GISWKIITIGYADSVKG

SEQ ID NO: 116 - protein, 17 aa, Synthetic construct
GISWKNGKIDYADSVKG

SEQ ID NO: 117 - protein, 17 aa, Synthetic construct
GISWNSNNIDYADSVKG

SEQ ID NO: 118 - protein, 16 aa, Synthetic construct
NIYYSGSTYYNPSLKS

SEQ ID NO: 119 - protein, 19 aa, Synthetic construct
RIKSKTDGGRIDYAAPVKG

SEQ ID NO: 120 - protein, 17 aa, Synthetic construct
SISSSSNYIYYVDSVKG

SEQ ID NO: 121 - protein, 17 aa, Synthetic construct
GISWKSGRIDYADSVKG

SEQ ID NO: 122 - protein, 18 aa, Synthetic construct
RTYFRSKWNNDYTLSVKN

SEQ ID NO: 123 - protein, 17 aa, Synthetic construct
GINWNGGSTGYVDSVKG

SEQ ID NO: 124 - protein, 10 aa, Synthetic construct
AKDSRTGLDY

SEQ ID NO: 125 - protein, 8 aa, Synthetic construct
TTVPYSDY

SEQ ID NO: 126 - protein, 7 aa, Synthetic construct
HNWSYGY

SEQ ID NO: 127 - protein, 8 aa, Synthetic construct
ARGRLFDY

SEQ ID NO: 128 - protein, 11 aa, Synthetic construct
AKDKGTTGFDY

SEQ ID NO: 129 - protein, 8 aa, Synthetic construct
DSTDGLDY

SEQ ID NO: 130 - protein, 8 aa, Synthetic construct
DTRSGLDY

SEQ ID NO: 131 - protein, 9 aa, Synthetic construct
DRREMTFDI

SEQ ID NO: 132 - protein, 8 aa, Synthetic construct
PGISSFEY

SEQ ID NO: 133 - protein, 19 aa, Synthetic construct
IGSSSEGVEDYYYYYYMDV

SEQ ID NO: 134 - protein, 12 aa, Synthetic construct
SGSHYYYNYMEV

SEQ ID NO: 135 - protein, 5 aa, Synthetic construct
EASNF

SEQ ID NO: 136 - protein, 8 aa, Synthetic construct
DTEAGLDY

SEQ ID NO: 137 - protein, 5 aa, Synthetic construct
ESGSP

SEQ ID NO: 138 - protein, 8 aa, Synthetic construct
DTSDGLDY

SEQ ID NO: 139 - protein, 12 aa, Synthetic construct
DSPQKDYYYMDV

SEQ ID NO: 140 - protein, 16 aa, Synthetic construct
QSTVVTSDYYYYYMDV

SEQ ID NO: 141 - protein, 12 aa, Synthetic construct
GLEPNYYYYMDV

SEQ ID NO: 142 - protein, 12 aa, Synthetic construct
AGYYYYYYYMDV

SEQ ID NO: 143 - protein, 5 aa, Synthetic construct
TPFDY

SEQ ID NO: 144 - protein, 5 aa, Synthetic construct
TNWNS

SEQ ID NO: 145 - protein, 11 aa, Synthetic construct
ECSGSYYRFDP

SEQ ID NO: 146 - protein, 19 aa, Synthetic construct
SDYYDSGSYKNYYYYYMDV

SEQ ID NO: 147 - protein, 17 aa, Synthetic construct
ENILTGYYNLYYYYMDV

SEQ ID NO: 148 - protein, 5 aa, Synthetic construct
EVGNY

SEQ ID NO: 149 - protein, 8 aa, Synthetic construct
DSRTGLDY

SEQ ID NO: 150 - protein, 9 aa, Synthetic construct
DRIEMTFDI

SEQ ID NO: 151 - protein, 12 aa, Synthetic construct
DGPGTTNDAFDI

SEQ ID NO: 152 - protein, 14 aa, Synthetic construct
HRGVTVTTYWYFDL

SEQ ID NO: 153 - protein, 6 aa, Synthetic construct
VPYSDY

SEQ ID NO: 154 - protein, 15 aa, Synthetic construct
QEATGNNYYYYYMDV

SEQ ID NO: 155 - protein, 9 aa, Synthetic construct
DRRELTFDI

SEQ ID NO: 156 - protein, 15 aa, Synthetic construct
GGYCNRTYCPDVFDI

SEQ ID NO: 157 - protein, 8 aa, Synthetic construct
MFYGAFDI

SEQ ID NO: 158 - protein, 11 aa, Synthetic construct
QSLVHSDGNTY

SEQ ID NO: 159 - protein, 12 aa, Synthetic construct
QSVLYSSDNENY

SEQ ID NO: 160 - protein, 9 aa, Synthetic construct
TGAVTSYYY

SEQ ID NO: 161 - protein, 12 aa, Synthetic construct
QSVLYSSNNKNY

SEQ ID NO: 162 - protein, 6 aa, Synthetic construct
QGISRW

SEQ ID NO: 163 - protein, 16 aa, Synthetic construct
RSSQSLLHSNGNTYLS

SEQ ID NO: 164 - protein, 16 aa, Synthetic construct
RSSQSLEHSDGNTYLS

SEQ ID NO: 165 - protein, 16 aa, Synthetic construct
RSSQSLEHSNGNTYLS

SEQ ID NO: 166 - protein, 11 aa, Synthetic construct
RATQDISNYLD

SEQ ID NO: 167 - protein, 11 aa, Synthetic construct
RASQNINSYLN

SEQ ID NO: 168 - protein, 12 aa, Synthetic construct
RASQTVSSSYLV

SEQ ID NO: 169 - protein, 11 aa, Synthetic construct
QASQDISNYLN

SEQ ID NO: 170 - protein, 16 aa, Synthetic construct
RSSQSLVHSNGNTYLS

SEQ ID NO: 171 - protein, 11 aa, Synthetic construct
RTSQSISSYLN

SEQ ID NO: 172 - protein, 11 aa, Synthetic construct
RASQSISNYLN

SEQ ID NO: 173 - protein, 12 aa, Synthetic construct
RASQSVSSSYLA

SEQ ID NO: 174 - protein, 16 aa, Synthetic construct
RSSQSLLHSNGYNYLD

SEQ ID NO: 175 - protein, 11 aa, Synthetic construct
QASQDISNHLN

SEQ ID NO: 176 - protein, 11 aa, Synthetic construct
QASQDISNYLH

SEQ ID NO: 177 - protein, 11 aa, Synthetic construct
RASQSISSYLN

SEQ ID NO: 178 - protein, 11 aa, Synthetic construct
QASQDIYNYLN

SEQ ID NO: 179 - protein, 11 aa, Synthetic construct
RASQTISSYLN

SEQ ID NO: 180 - protein, 11 aa, Synthetic construct
RASHSISSYLN

SEQ ID NO: 181 - protein, 11 aa, Synthetic construct
RASQSIGNSLH

SEQ ID NO: 182 - protein, 17 aa, Synthetic construct
KSSQSVLYSSDNENYLA

SEQ ID NO: 183 - protein, 11 aa, Synthetic construct
RSSQSISSYLN

SEQ ID NO: 184 - protein, 12 aa, Synthetic construct
RASQSVSSNYLA

SEQ ID NO: 185 - protein, 7 aa, Synthetic construct
KISNRFS

SEQ ID NO: 186 - protein, 7 aa, Synthetic construct
DASNLET

SEQ ID NO: 187 - protein, 7 aa, Synthetic construct
AASSLQS

SEQ ID NO: 188 - protein, 7 aa, Synthetic construct
GASGRAT

SEQ ID NO: 189 - protein, 7 aa, Synthetic construct
DASNLER

SEQ ID NO: 190 - protein, 7 aa, Synthetic construct
GASTRAT

SEQ ID NO: 191 - protein, 7 aa, Synthetic construct
LGSNRAS

SEQ ID NO: 192 - protein, 7 aa, Synthetic construct
KISHRFS

SEQ ID NO: 193 - protein, 7 aa, Synthetic construct
AASSLQR

SEQ ID NO: 194 - protein, 7 aa, Synthetic construct
YVSQSFS

SEQ ID NO: 195 - protein, 7 aa, Synthetic construct
WASSRES

SEQ ID NO: 196 - protein, 7 aa, Synthetic construct
VASTLQS

SEQ ID NO: 197 - protein, 7 aa, Synthetic construct
GASSRAT

SEQ ID NO: 198 - protein, 9 aa, Synthetic construct
MQATQFPYT

SEQ ID NO: 199 - protein, 9 aa, Synthetic construct
HQYYNTPWT

SEQ ID NO: 200 - protein, 9 aa, Synthetic construct
LLFYGGAYV

SEQ ID NO: 201 - protein, 10 aa, Synthetic construct
QQYYSTPSYT

SEQ ID NO: 202 - protein, 9 aa, Synthetic construct
QQANSFPRT

SEQ ID NO: 203 - protein, 9 aa, Synthetic construct
MQATEFPYT

SEQ ID NO: 204 - protein, 9 aa, Synthetic construct
MQATEFPFT

SEQ ID NO: 205 - protein, 9 aa, Synthetic construct
MQATQFPLT

SEQ ID NO: 206 - protein, 9 aa, Synthetic construct
QQYDNLPLT

SEQ ID NO: 207 - protein, 9 aa, Synthetic construct
HQSYSTPLT

SEQ ID NO: 208 - protein, 9 aa, Synthetic construct
QHYGSSPYT

SEQ ID NO: 209 - protein, 9 aa, Synthetic construct
QQYESLPFN

SEQ ID NO: 210 - protein, 9 aa, Synthetic construct
QEYDSLPFT

SEQ ID NO: 211 - protein, 9 aa, Synthetic construct
MQAIQFPYT

SEQ ID NO: 212 - protein, 9 aa, Synthetic construct
QQSYITPIT

SEQ ID NO: 213 - protein, 9 aa, Synthetic construct
QQSYSPPIT

SEQ ID NO: 214 - protein, 9 aa, Synthetic construct
QQYNNWPLT

SEQ ID NO: 215 - protein, 8 aa, Synthetic construct
MQALQTYT

SEQ ID NO: 216 - protein, 9 aa, Synthetic construct
QQYDNVPYT

SEQ ID NO: 217 - protein, 9 aa, Synthetic construct
QHYDNLPLT

SEQ ID NO: 218 - protein, 9 aa, Synthetic construct
QQSYSTPLT

SEQ ID NO: 219 - protein, 7 aa, Synthetic construct
QQSYSTS

SEQ ID NO: 220 - protein, 9 aa, Synthetic construct
QQYDSLPIT

SEQ ID NO: 221 - protein, 9 aa, Synthetic construct
QQTYTAPLT

SEQ ID NO: 222 - protein, 9 aa, Synthetic construct
MQTTQFPLT

SEQ ID NO: 223 - protein, 9 aa, Synthetic construct
HQGYSTPLT

SEQ ID NO: 224 - protein, 9 aa, Synthetic construct
HQSSDFPHT

SEQ ID NO: 225 - protein, 9 aa, Synthetic construct
QQTYSTPIT

SEQ ID NO: 226 - protein, 9 aa, Synthetic construct
QQSYSIPLT

SEQ ID NO: 227 - protein, 9 aa, Synthetic construct
QQYGSSPFT

SEQ ID NO: 228 - protein, 330 aa, Synthetic construct [sequence omitted]

SEQ ID NO: 229 - protein, 327 aa, Synthetic construct
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 230 - protein, 107 aa, Synthetic construct [sequence omitted]

SEQ ID NO: 231 - protein, 106 aa, Synthetic construct
GQPKANPTVTLFPPSSEELQANKATLVCLISDFYPGAVTVAWKADGSPVKAGVETTKPSKQSNNKYAASSYLSLTPEQWKSHRSYSCQVTHEGSTVEKTVAPTECS

SEQ ID NO: 232 - protein, 165 aa, Homo sapiens
MALLALLLVVALPRVWTDANLTARQRDPEDSQRTDEGDNRVWCHVCERENTFECQNPRRCKWTEPYCVIAAVKIFPRFFMVAKQCSAGCAAMERPKPEEKRFLLEEPMPFFYLKCCKIRYCNLEGPPINSSVFKEYAGSMGESCGGLWLAILLLLASIAAGLSLS

SEQ ID NO: 233 - protein, 15 aa, Synthetic construct
WTDANLTARQRDPED

SEQ ID NO: 234 - protein, 9 aa, Synthetic construct
LTARQRDPE

SEQ ID NO: 235 - protein, 10 aa, Synthetic construct
QRDPEDSQRT

SEQ ID NO: 236 - protein, 12 aa, Synthetic construct
DANLTARQRDPE

SEQ ID NO: 237 - protein, 10 aa, Synthetic construct
FECQNPRRCK

SEQ ID NO: 238 - protein, 10 aa, Synthetic construct
PEEKRFLLEE

SEQ ID NO: 239 - protein, 15 aa, Synthetic construct
WTHLNLTARQQDPED

SEQ ID NO: 240 - protein, 9 aa, Synthetic construct
LTARQQDPE

SEQ ID NO: 241 - protein, 15 aa, Synthetic construct
KTRHPLEMVILLALL

SEQ ID NO: 242 - protein, 9 aa, Synthetic construct
LEMVILLAL

SEQ ID NO: 243 - protein, 15 aa, Synthetic construct
QSNALTCHVCEAQNS

SEQ ID NO: 244 - protein, 9 aa, Synthetic construct
TCHVCEAQN

SEQ ID NO: 245 - protein, 10 aa, Synthetic construct
QQDPEDTPQT

SEQ ID NO: 246 - protein, 12 aa, Synthetic construct
HLNLTARQQDPE

SEQ ID NO: 247 - protein, 10 aa, Synthetic construct
FECENPRRCQ